(19) United States
(12) Patent Application Publication
(10) Pub. No.: US 2005/0074850 A1
(43) Pub. Date: Apr. 7, 2005

(54) Novel calcium channels and uses thereof

(75) Inventors: Monica J.S. Nadler, Framingham, MA (US); Jean-Pierre Kinet, Lexington, MA (US); Pierre Launay, Brighton, MA (US); Jerome Mahiou, Lexington, MA (US)

Correspondence Address: WILMER CUTLER PICKERING HALE AND DORR LLP, 60 STATE STREET, BOSTON, MA, 02109, US

(73) Assignees: Synta Pharmaceuticals Corp., Lexington, MA (US); Beth Israel Deaconess Medical Center, Inc., Boston, MA (US)

(21) Appl. No.: 10/856,122

(22) Filed: May 28, 2004

Related U.S. Application Data
(60) Provisional application No. 60/474,245, filed on May 28, 2003.

(51) Int. Cl. C07K 14/705; C07H 21/04

(52) U.S. Cl. 435/69.1; 435/320.1; 435/325; 530/350; 536/23.5

(57) ABSTRACT

Differentially expressed L-type calcium channel nucleic acid sequences and polypeptides have been identified that include novel sequences that are differentially expressed in non-excitable cells. Such sequences can be used, for example, as targets for identifying agents that modulate calcium influx, e.g., in non-excitable cells. Methods related to modulation of cell growth are also included.

### CROSS REFERENCE TO RELATED APPLICATION

This application claims priority from U.S. Provisional Application Ser. No. 60/474,245, filed May 28, 2003, which is incorporated herein by reference in its entirety.

### TECHNICAL FIELD

This invention relates to calcium channels.

### BACKGROUND

Regulation of calcium concentration plays an important role in many cellular processes. Changes in the cytoplasmic free calcium ion concentration ([Ca2+]c) constitute one of the main pathways by which information is transferred from extracellular signals received by animal cells to intracellular sites. The intracellular Ca2+ signal is conveyed by the magnitude, location, and duration of the changes in [Ca2+]c. Increases in [Ca2+]c in a given region of the cytoplasmic space can be initiated by the binding of an extracellular signaling molecule (agonist) to its plasma-membrane receptors. Such signals typically arise from both the release of stored calcium and the influx of calcium across the plasma membrane. This is a fundamental property of almost any given cell. However, the channels and mechanisms that govern this process vary amongst cell types.

Cells of the immune system are considered non-excitable. In other words, these cells do not respond to changes in voltage that are characteristic of cells in the cardiac, nervous, and neuroendocrine systems. In immune system cells, calcium flux is paramount for many processes in lymphocytes including cell growth, differentiation and effector functions. Once a lymphocyte or mast cell is activated, intracellular concentrations of calcium ions are increased by both a release from intracellular stores and by entry from the extracellular milieu via a specific set of channels in the cellular membrane. This process is commonly referred to as capacitative calcium influx, or store-operated calcium influx.

On the molecular level, a common mechanism by which such cytoplasmic calcium signals are generated involves receptors that are coupled to the activation of phospholipase C. In electrically non-excitable mammalian cells, activation of phosphoinositide-specific phospholipase C produces inositol 1,4,5-trisphosphate (IP3), which in turn triggers the release of intracellular calcium from intracellular stores (most commonly, components of the endoplasmic reticulum), thereby allowing calcium to be released into the cytosol. This results in a transient elevation of cytosolic free Ca2+, which is normally followed by Ca2+ influx from the extracellular space. In most cells, the fall in [Ca2+]c within the lumen of the Ca2+-storing organelles subsequently activates plasma membrane Ca2+ channels. Such Ca2+ influx plays an important role in prolonging the Ca2+ signal, allowing for localized signaling, and maintaining Ca2+ oscillations (Berridge, 1993, Nature 361: 315-325).

Store-operated calcium influx is believed to be the most fundamental mechanism of calcium influx in non-excitable cells of the immune system (for example, blood cells, epithelial cells, immune cells (such as T cells, B cells, mast cells, macrophages and monocytes) and tumor cells). As a second messenger, calcium influx via capacitative calcium entry can induce short term cellular responses, such as protein-protein interactions and granule secretion, and can also initiate longer-term cellular control mechanisms, such as gene transcription that supports cell growth, apoptosis, differentiation, or activation. In in vitro studies, the effect of this necessary calcium signal for activation of genes transcription factor can be induced by the action of calcium ionophores, such as ionomycin (see Putney, 2001, The Pharmacology of Capacitative Calcium Entry, Molecular Interventions 1:84-94).

The store-operated calcium influx process generates a calcium-selective current in lymphocytes and mast cells termed Icrac (calcium release activated calcium current). Although Icrac currents have been known to exist for some time, the molecular identity of the channel(s) responsible for calcium influx in lymphocytes and mast cells, and in particular Icrac currents, remains unknown. Some have postulated that store-operated calcium influx is mediated by members of the TRP family of ion channels and the epithelial-related channel CAT1 (Cui et al., 2002, J. Biol. Chem. 377: 47175-47183). However, there is recent conflicting data in the field regarding the contribution of CAT1 channels to Icrac currents, with no clear consensus that CAT1 is responsible for Icrac currents (Voets et al., 2001, J. Biol. Chem. 276: 47767-47770). Furthermore, another TRP family member, TRP1, has been reported to impact Icrac currents (Mori et al., 2002, J. Exp. Med. 195: 673-681). Thus, an active role for TRP1 for Icrac currents remains debated.

It is also notable that other less-defined modes of active calcium flux are thought to functionally exist in cells of the immune system. These include diacyl-glycerol mediated (DAG) calcium entry, which may be mediated by a TRP family member. However, this form of entry is not well established in immune system cells. Another mode includes the CD38 receptor system. This receptor has a well-established effect on calcium influx, having a unique ADP-ribose cyclase activity. However, it is not understood how this receptor functions to affect influx. Finally, there are cases of apoptotic signaling which may or may not involve ADP-ribose. Clearly, the entry mechanisms for these processes are not well understood. It should also be noted that other calcium currents in addition to Icrac have been observed in lymphocytes and mast cells. Although these currents can be distinguished from Icrac, their molecular identification will provide an enlightening view of the molecular complexity that underlies calcium influx and homeostasis in cells of the immune system.

Voltage-dependent calcium channels (VDCCs or VOCCs) are a large family of channels found in all excitable tissues and some non-excitable cell types (Catterall, 2000, Ann. Rev. Cell. Dev. Biol. 16: 521-555). They are formed as a molecular complex with accessory subunits; the large α-subunit forms the ion conduction pore and the α2-δ-, β and γ subunits are the accessory subunits that modulate a function (Catterall, 2000, supra). These subunits have been implicated in efficient a expression as well as current modulation. The α subunit forms the minimal operational structure of the channel complex. In addition to forming the pore, it harbors the voltage sensor, binding sites for the accessory subunits, binding sites for calcium channel modulators, and binding sites for mediators within intracellular signaling pathways. The VDCC family α subunits are classified into three main families on the basis of their electrophysiolgical profiles and sequence homology, Ca(v)1.x, Ca(v)2.x and Ca(v)3.x (Catterall, 2000, supra).

### SUMMARY

The invention is based, in part, on the discovery of calcium channel subunits that are differentially expressed in non-excitable cells, e.g., lymphocytes. The novel channel proteins that are expressed in non-excitable cells are variants of a member of the L-type channel family (the variants are herein termed LVCa(v)), and are associated with calcium influx in non-excitable cells (e.g., non-voltage-gated calcium influx). Accordingly, the LVCa(v) nucleic acid sequences include SEQ ID NO:1 (novel exon A of LVCa(v)1.3), SEQ ID NO:3 (novel exon B of LVCa(v)1.3), SEQ ID NO:5 (novel exon A/B sequence of LVCa(v)1.3), SEQ ID NO:9 (novel exon 33 of LVCa(v)1.3, termed herein exon 33a), SEQ ID NO:11 (novel exon created by the absence of exon 33 and the joining of exon 32 and 34), SEQ ID NO:13 (novel sequence created by the absence of exon 12 and thus conjoining exons 11 and 13), LVCa(v)1.3 sequences that terminate at the end of exon 44 (SEQ ID NO:15 depicts the sequence of exon 44), SEQ ID NO:17 (the coding region of LV1Ca(v)1.3.1/12/33 which contains exon A/B, lacks exon 12, contains exon 33a, and terminates after exon 44), SEQ ID NO:18 (LV1Ca(v)1.3, 1/12/33, which is a full length cDNA sequence including 5′ and 3′ untranslated regions), SEQ ID NO:20 (LV2Ca(v)1.3 cDNA showing the coding region which includes exon A/B, exon 12, exon 33a, and terminates after exon 44), SEQ ID NO:23 (exon A′, novel first exon of LVCa(v)1.1), SEQ ID NO:25 (exon B′, novel second exon of LVCa(v)1.1), SEQ ID NO:26 (exon A′/B′, which encodes the amino terminus of LVCa(v)1.1 (combined exons A′ and B′)).

LVCa(v) amino acid sequences include SEQ ID NO:2 (novel exon A of LVCa(v)1.3), SEQ ID NO:4 (novel exon B of LVCa(v)1.3), SEQ ID NO:6 (novel exon A/B sequence (combined A and B) of LVCa(v)1.3), SEQ ID NO:7 (novel exon A/B sequence (combined A and B) alternate start site beginning at the second methionine encoded by SEQ ID NO:5, SEQ ID NO:8 (novel exon A/B sequence with allelic variation; MFYIMMEPLFRCRKTSSRLPLILHD), SEQ ID NO:10 (novel exon 33, termed exon 33a), SEQ ID NO:12 (novel exon created by the absence of exon 33 and the joining of exon 32 and 34), SEQ ID NO:14 (novel sequence created by the absence of exon 12 and thus conjoining exons 11 and 13), LVCa(v)1.3 sequence that terminates after exon 44 (SEQ ID NO:16 shows the amino acid sequence of exon 44, SEQ ID NO:19 (LV1Ca(v), 1/12/33, which has exon A/B and lacks exons 12 and 33 of the Ca(v)1.3 gene), SEQ ID NO: 22 (LV2Ca(v)1.3 which includes exons A/B, exon 12, exon 33a, and terminates after exon 44), SEQ ID NO:24 (exon A′, novel first exon of LVCa(v)1.1), SEQ ID NO:27 (polypeptide sequence encoding the novel amino terminus of the LVCa(v)1.1 (combined exons A′ and B′)) and a variant of LV2Ca(v)1.3 that includes exon 12.

LVCa(v) polypeptides and nucleic acid sequences are useful for, e.g., identifying compounds that modulate expression or activity of these sequences, and for uses related to treating T cell, B cell, and mast cell related diseases such as T cell and B cell lymphomas and tumors, inflammatory disorders, and autoimmune disorders, asthma, allergic disorders, T cell-mediated transplant rejection, graft rejection, and graft versus host diseases.

Accordingly, the invention relates to an isolated nucleic acid molecule encoding a polypeptide, such that the polypeptide comprises an amino acid sequence selected from the group consisting of the amino acid sequence encoded by exon A, exon B, exon A/B, exon 11-13, exon 33A, exon 32-34, LV1Ca(v)1.3, LV2Ca(v)1.3, exon A′, exon B′, exon A′/B′, an LVCa(v)1.3 amino acid sequence terminating at the end of exon 44, an amino acid sequence terminating at the end of Exon 50, and variants thereof having one or more conservative amino acid substitutions. The isolated nucleic acid sequence can be from a mammal, e.g., a human, rat, mouse, rabbit, goat, cow, pig. The invention also features an isolated nucleic acid molecule, the nucleic acid molecule including a nucleic acid sequence selected from the group consisting of exon A, exon B, exon A/B, exon 11-13, exon 33A, exon 32-34, a LV1Ca(v)1.3 nucleic acid molecule, an LV2Ca(v)1.3 nucleic acid molecule, exon A′, exon B′, exon A′/B′, a nucleic acid molecule encoding an LVCa(v)1.3 amino acid sequence terminating at the end of exon 44, a nucleic acid molecule encoding an amino acid sequence terminating at the end of exon 50, and variants thereof having one or more substitutions resulting in conservative amino acid substitutions; or a complement thereof.

An isolated nucleic acid molecule including a polynucleotide sequence that hybridizes to a second polynucleotide sequence selected from the group consisting of exon A, exon B, exon A/B, exon 11-13, exon 33A, exon 32-34, LV1Ca(v)1.3, LV2Ca(v)1.3, exon A′, exon B′, exon A′/B′, a sequence encoding an LVCa(v)1.3 amino acid sequence terminating at the end of exon 44 or a portion thereof, a sequence encoding an amino acid sequence terminating at the end of exon 50, a 3′ untranslated sequence of an LVCa(v)1.3, a 5′ untranslated sequence of an LVCa(v)1.3, a 5′ untranslated sequence of an LVCa(1.1), and variants thereof having one or more substitutions resulting in conservative amino acid substitutions; or a complement thereof, the hybridization conditions including hybridization in 50% formamide at 42° C. and washing in 0.2×SSC and 0.1% SDS at 68° C.

The invention includes a recombinant expression vector including a nucleic acid molecule described herein, e.g., an LVCa(v) nucleic acid molecule such as an LVCa(1.3) or an LVCa(1.1) molecule. Also included is a recombinant cultured cell including a nucleic acid molecule described herein, for example, a recombinant cultured cell including a nucleic acid molecule described herein, such that expression of the nucleic acid molecule increases cell growth compared to a control cell.

Also featured is a recombinant cultured cell including a polypeptide encoded by a nucleic acid sequence consisting of exon A, exon B, exon A/B, exon 11-13, exon 33A, exon 32-34, an LV1Ca(v)1.3 nucleic acid molecule, an LV2Ca(v)1.3 nucleic acid molecule, exon A′, exon B′, exon A′/B′, a nucleic acid molecule encoding an LVCa(v)1.3 amino acid sequence terminating at the end of exon 44, a nucleic acid molecule encoding an LVCa(v) amino acid sequence terminating at the end of exon 50, or variants thereof having one or more conservative amino acid substitutions. In some cases, the recombinant cultured cell has increased cell growth compared to a control cell that does not comprise (express) the polypeptide. In some embodiments, a recombinant cultured cell is featured that includes a deletion in at least one allele of an LVCa(v) nucleic acid sequence, such that the level of expression of the LVCa(v) polypeptide is reduced compared to a cell without the deletion, and such that the polypeptide comprises an amino acid sequence consisting of exon A, exon B, exon A/B, exon 11-13, exon 33A, exon 32-34, LV1Ca(v)1.3, LV2Ca(v)1.3, exon A′, exon B′, exon A′/B′, an LVCa(v)1.3 amino acid sequence terminating at the end of exon 44, an amino acid sequence terminating at the end of exon 50, or variants thereof having one or more conservative amino acid substitutions. Also included is a recombinant cultured cell including a deletion in at least one allele of a LVCa(v) nucleic acid sequence, such that the level of expression of the LVCa(v) nucleic acid sequence is reduced compared to a cell without the deletion, and such that the gene comprises a nucleic acid sequence including a sequence consisting of exon A, exon B, exon A/B, exon 11-13, exon 33A, exon 32-34, an LV1Ca(v)1.3 nucleic acid molecule, an LV2Ca(v)1.3 nucleic acid molecule, exon A′, exon B′, exon A′/B′, a sequence encoding an LVCa(v)1.3 amino acid sequence terminating at the end of exon 44 or a portion thereof, a sequence encoding an amino acid sequence terminating at the end of exon 50, a 3′ untranslated sequence of an LVCa(v)1.3, a 5′ untranslated sequence of an LVCa(v)1.3, a 5′ untranslated sequence of an LVCa(1.1), variants thereof having one or more substitutions resulting in conservative amino acid substitutions; and a complement thereof. The recombinant cultured cell can be a cultured immune system cell, e.g., a T cell, such as a DT40 cell or a Jurkat cell.

The invention also relates to a substantially pure polypeptide including an amino acid sequence that contains exon A, exon B, an exon A/B, exon 11-13, exon 33A, exon 32-34, LV1Ca(v)1.3, LV2Ca(v)1.3, exon A′, exon B′, exon A′/B′, an LVCa(v)1.3 amino acid sequence terminating at the end of exon 44, an amino acid sequence terminating at the end of exon 50, or variants thereof having one or more conservative amino acid substitutions. The polypeptide can consist of the amino acid sequence of exon A, exon B, exon A/B, exon 11-13, exon 33A, exon 32-34, LV1Ca(v)1.3, LV2Ca(v)1.3, exon A′, exon B′, exon A′/B′, an LVCa(v)1.3 amino acid sequence terminating at the end of exon 44 (SEQ ID NO. 2), an amino acid sequence terminating at the end of exon 50, or variants thereof having one of more conservative amino acid substitutions. In some cases, the polypeptide can exhibit LVCa(v) activity, e.g., can increase cell growth (proliferation) when expressed in a cell, or can increase calcium influx under suitable conditions. Recombinant expression of the polypeptide in a cell can, in some cases, modulate cell growth compared to a control.

The invention also features a substantially pure polypeptide encoded by a nucleic acid sequence that hybridizes to any of exon A, exon B, exon A/B, exon 11-13, exon 33A, exon 32-34, LV1Ca(v)1.3, LV2Ca(v)1.3, exon A′, exon B′, exon A′/B′, a sequence encoding an LVCa(v)1.3 amino acid sequence terminating at the end of Exon 44 or a portion thereof, a sequence encoding an amino acid sequence terminating at the end of Exon 50, a 3′ untranslated sequence of an LVCa(v)1.3, a 5′ untranslated sequence of an LVCa(v)1.3, a 5′ untranslated sequence of an LVCa(1.1), and variants thereof having one or more substitutions resulting in conservative amino acid substitutions, or a complement thereof, the hybridization conditions including hybridization in 50% formamide at 42° C. and washing in 0.2×SSC and 0.1% SDS at 68° C.

The invention also relates to a method for identifying an agent that modulates expression of an LVCa(v) gene in a cell, the method includes obtaining a test cell that expresses an LVCa(v) polypeptide; contacting the test cell with a test agent; measuring the level of expression of the LVCa(v) mRNA in the test sample exposed to the test agent; determining that the test agent is a modulator of LVCa(v) expression if the level of expression of the LVCa(v) mRNA in the test sample exposed to the test agent is less than the level of expression of the LVCa(v) in a test cell that was not contacted with the test agent. In some cases, the cell is a non-excitable cell, e.g., the cell is an immune system cell such as a B cell, T cell, or mast cell. In some embodiments, the LVCa(v) is an LVCa(v)1.3. The method can include contacting the test sample with a nucleic acid molecule that hybridizes to the LVCa(v) mRNA under stringent conditions, e.g., when the test agent is an antisense agent or an RNAi agent.

Also included is a method for identifying an agent that modulates expression of an LVCa(v) polypeptide in a cell, the method includes obtaining a test cell that expresses an LVCa(v) polypeptide; contacting the test cell with a test agent; measuring the level of expression of the LVCa(v) polypeptide in the test cell contacted with the test agent; determining that the test agent is an agent that modulates expression of the LVCa(v) polypeptide if the level of expression of the LVCa(v) polypeptide in the test sample contacted with the test agent is less than the level of expression in a test cell that was not contacted with the test agent. In some embodiments, the cell is a non-excitable cell. The method can be performed such that the test sample is contacted with an agent that binds to the LVCa(v) polypeptide (e.g., an LVCa(v)1.3 or an LVCa(v)1.1 polypeptide. In some cases, the test agent is an antibody, e.g., a monoclonal antibody, a single chain antibody, a Fab, or an epitope-binding fragment of an antibody. The test agent can be detectably labeled, for example, with a radioactive label, a fluorescent label, a chemiluminescent label, or a bioluminescent label.

The invention includes a method for identifying an agent that modulates activity of an LVCa(v) polypeptide in a cell. The method includes obtaining a test sample including a cell that expresses an LVCa(v) polypeptide; contacting the test sample with a test agent; measuring the level of activity of the LVCa(v) polypeptide in the test sample contacted with the test agent; determining that the test agent is an agent that modulates an LVCa(v) activity if the level of activity of the LVCa(v) polypeptide in the test sample contacted with the test agent is less than the level of expression in test sample that was not contacted with the test agent. In the method, the test agent can be a dihydropyridine, phenylalkylamine, benzodiazepine, benzothiazapine, diarylaminopropylamine ether, or benzimidazole-substituted tetralin. The test agent can, in some cases inhibit the activity of the LVCa(v) polypeptide in vitro by at least about 50% at a concentration of about 1 μM-100 μM, less than about 50 μM, less than about 10 μM, or less than about 1 μM. The activity of the LVCa(v) polypeptide can be modulation (e.g., increasing or decreasing) cell growth. In some cases, the activity of the LVCa(v) polypeptide is modulation of calcium flux, or the test agent can inhibits phosphorylation of the LVCa(v). A test agent can affect more than one activity. The LVCa(v) can be an LVCa(v)1.3 polypeptide, e.g., an LV1Ca(v)1.3 polypeptide or an LV2Ca(v)1.3 polypeptide.

The invention features a method of inhibiting calcium influx in a non-excitable cell. The method includes inhibiting the activity of an LVCa(v) polypeptide that is expressed in the non-excitable cell. For example, the activity of the LVCa(v) polypeptide can be increased cell growth. The LVCa(v) polypeptide can be an LVCa(v)1.3 polypeptide.

Also included in the invention is a method of inhibiting calcineurin activity in a non-excitable cell. The method includes inhibiting the activity of an LVCa(v) polypeptide that is expressed in the non-excitable cell. In some cases, embodiment relates to a method of inhibiting NFAT activity in a non-excitable cell. The method includes inhibiting activity of an LVCa(v) polypeptide that is expressed in the non-excitable cell. Also featured is a method of inhibiting IL-2 production in a non-excitable cell. The method includes inhibiting the activity of an LVCa(v) polypeptide that is in the non-excitable cell. In some cases, the invention relates to a method of inhibiting secretion of a cytokine in a non-excitable cell such as a T cell, B cell, lymphocyte, a mast cell, an HEK293 cell, or a Jurkat cell, and the method includes inhibiting the expression or activity of an LVCa(v) polypeptide in the non-excitable cell.

The invention also relates to a method of inhibiting the activity of a Ca²⁺-activated gene product in a non-excitable cell. The method includes the step of inhibiting the activity of an LVCa(v) polypeptide in the non-excitable cell. Also featured is a method of inhibiting proliferation of a non-excitable cell. The method includes selectively inhibiting the activity of an LVCa(v) polypeptide in the non-excitable cell, or example, phosphorylation of the LVCa(v) polypeptide is inhibited. The non-excitable cell can be, e.g., a cancer cell or other cell characteristic of a proliferative cell disorder.

In some embodiments, the invention is a method of inhibiting differentiation of a non-excitable cell. The method includes inhibiting the activity of an LVCa(v) polypeptide in the non-excitable cell, e.g., such that phosphorylation of the LVCa(v) polypeptide is inhibited.

Also featured is a method of inhibiting immune cell function. The method includes inhibiting at least one activity of an LVCa(v) polypeptide in the cell, for example, calcium flux is inhibited or cell proliferation is inhibited.

In any of the methods described herein, the activity of the LVCa(v) polypeptide can, in some cases, be inhibited in vitro by at least about 50% using an agent that is present at a concentration of less than about 1-100 μM, less than about 50 μM, less than about 10 μM, or less than about 1 μM.

In any of the methods described herein, a non-nucleic acid agent (e.g., a test agent) can be a dihydropyridine, phenylalkylamine, benzodiazepine, benzothiazapine, diarylaminopropylamine ether, benzimidazole-substituted tetralin, or a derivative thereof.

In any of the methods described herein, the LVCa(v) polypeptide can be an LVCa(v)1.3 polypeptide, e.g., an LVCa(v)1.3 polypeptide that contains one or more of the amino acid sequences depicted in FIGS. 1B, 1D, 2B, 2D, 2F, 3B, 4B, 6B, 8B, an amino acid sequence terminating at the end of a Ca(v)1.3 exon 44, an amino acid sequence terminating at the end of a Ca(v)1.3 exon 50, or variants thereof having one or more conservative amino acid substitutions.

In any of the methods described herein in which a non-excitable cell is employed, the non-excitable cell can be an immune system cell such as a lymphocyte, mast cell, and a cell derived from a lymphocyte or mast cell (for example, a T cell, a B cell, or a DT40 chicken cell). In some cases the non-excitable cell is from a cell line such as a T cell line, for example, a Jurkat cell.

The invention relates to a method for treating or preventing a cancer, an immune system disorder, or an inflammatory condition in a subject. The method includes inhibiting expression or activity of an LVCa(v) polypeptide that is expressed in a non-excitable cell. The immune system disorder can be, e.g., an allergic disorder, an immune system-related cancer, or an autoimmune disorder such as multiple sclerosis, myasthenia gravis, autoimmune neuropathies, Guillain-Barré, autoimmune uveitis, autoimmune hemolytic anemia, pernicious anemia, autoimmune thrombocytopenia, temporal arteritis, anti-phospholipid syndrome, vasculitides, Wegener's granulomatosis, Behcet's disease, psoriasis, dermatitis herpetiformis, pemphigus vulgaris, vitiligo, Crohn's disease, ulcerative colitis, primary biliary cirrhosis, and autoimmune hepatitis, Type 1 or immune-mediated diabetes mellitus, Grave's disease, Hashimoto's thyroiditis, autoimmune oophoritis and orchitis, autoimmune disease of the adrenal gland; rheumatoid arthritis, systemic lupus erythematosus, scleroderma, polymyositis, dermatomyositis, ankylosing spondylitis, Sjogren's syndrome and graft-versus-host disease. In some cases, the disorder is an immune system-related cancer that is selected from the group consisting of Kaposi's sarcoma and leukemia. In some embodiments, the activity of the LVCa(v) polypeptide is inhibited and inhibition in vitro is at least about 50% using an agent that is present at a concentration of less than about 1-100 μM, less than about 50 μM, less than about 10 μM, or less than about 1 μM. The agent is, in some cases, a dihydropyridine, phenylalkylamine, benzodiazepine, benzothiazapine, diarylaminopropylamine ether, benzimidazole-substituted tetralin, or a derivative thereof. The LVCa(v) polypeptide can be an LVCa(v)1.3 polypeptide, e.g., an LVCa(v)1.3 polypeptide that contains at least one an amino acid sequence depicted in any one of FIGS. 1B, 1D, 2B, 2D, 2F, 3B, 4B, 6B, 8B, an amino acid sequence terminating at the end of an exon 44, an amino acid sequence terminating at the end of an exon 50, or variants thereof having one or more conservative amino acid substitutions. The non-excitable cell can be a tumor cell, lymphocyte, mast cell, or a cell derived from a lymphocyte or mast cell, or a cell line derived from a non-excitable cell that can overexpress an LVCa(v) polypeptide, for example, a cell line such that the LVCa(v) polypeptide is an LVCa(v)1.3 polypeptide. The LVCa(v)1.3 polypeptide can include an amino acid sequence depicted in FIGS. 1B, 1D, 2B, 2D, 2F, 3B, 4B, 6B, 8B, an amino acid sequence terminating at the end of exon 44 (SEQ ID NO. 2), an amino acid sequence terminating at the end of exon 50 (SEQ ID NO. 3), or variants thereof having one or more conservative amino acid substitutions. The invention includes an isolated LVCa(v) polypeptide produced by cell as described herein.

The invention also relates to a method for identifying an LVCa(v) polypeptide that is differentially expressed in two or more non-excitable cell types, including quantitatively measuring the amount of mRNA encoding different LVCa(v) polypeptides in each cell type. This method can further include determining the expression profile of the LVCa(v) polypeptides in each cell type.

Also featured is a method for identifying a candidate modulator of an LVCa(v) polypeptide in a non-excitable cell. The method includes providing a non-excitable cell that can express one or more LVCa(v) polypeptides; contacting the cell with a test agent; measuring the ability of the test agent to inhibit calcium influx modulated by one or more LVCa(v) polypeptides that are differentially expressed in the cell, such that a test agent that inhibits calcium influx modulated by one or more LVCa(v) polypeptides in the cell is a candidate modulator of an LVCa(v) polypeptide. In some embodiments, the differential expression occurs between two or more different tissue types, e.g., thymus tissue and spleen tissue. In some cases, the differential expression occurs between two or more different cell types, e.g., T cells, mast cells, and B cells. The ability of the test agent to inhibit calcium influx is, in some cases, measured by assaying for one or more of the following activities: calcineurin activity, NFAT activity, or IL-2 activity. In some embodiments, expression of the LVCa(v) polypeptide is decreased when the cell is activated. In other embodiments, expression of the LVCa(v) polypeptide is increased when the cell is deactivated. Expression of the LVCa(v) polypeptide is, in some cases, modulated when the cell is undergoing differentiation. The differentially expressed LVCa(v) polypeptides can be identified using quantitative PCR.

The invention also relates to a method of screening for a modulator of an LVCa(v) polypeptide in a cell. The method includes providing a non-excitable cell that can express one or more LVCa(v) polypeptides; contacting the cell with a test agent; and evaluating the ability of the test agent to inhibit calcium influx modulated by one or more of the LVCa(v) polypeptides, such that inhibition of calcium influx in the presence of the test agent compared to a reference that was not contacted with the test agent indicates that the test agent is a modulator of an LVCa(v) polypeptide. The ability of the test agent to inhibit calcium influx is, in some cases, measured in a cell line that overexpresses an LVCa(v)1.3 polypeptide. In some embodiments, the cell expresses at least two different LVCa(v) polypeptides that are differentially expressed between two or more different tissue types, e.g., thymus and spleen. In yet other embodiments, at least two LVCa(v) polypeptides are expressed and the LVCa(v) polypeptides are differentially expressed between two or more different cell types, for example one or more of the LVCa(v) polypeptides are differentially expressed between a tumor cell and a normal cell, or the cell types are, e.g., T cells, mast cells, or B cells. In some embodiments, the LVCa(v) polypeptide is differentially expressed when the cell is activated compared to a cell that is not activated.

The invention also relates to a method for identifying a modified agent that can modulate the activity of an LVCa(v) polypeptide in a cell. The method includes providing an agent that modulates the activity of an LVCa(v) polypeptide in a cell; modifying the agent by producing a chemical analog or derivative thereof, thereby producing a modified agent; and measuring the ability of the modified agent to modulate the activity of an LVCa(v) polypeptide in a non-excitable cell, such that increased modulation in the presence of the modified agent compared to the agent indicates that the modified agent is an improved agent. In some cases, the modified agent modulates the LVCa(v) polypeptide in a non-excitable cell at or below a chosen threshold level, e.g., the threshold level is 50% inhibition of the LVCa(v) polypeptide in vitro at about 1 μM, or the threshold level is 50% inhibition of the LVCa(v) polypeptide in vitro at about 100 nM. The agent can be, e.g., a dihydropyridine, phenylalkylamine, benzodiazepine, benzothiazapine, diarylaminopropylamine ether, benzimidazole-substituted tetralin, or a derivative thereof. In some embodiments, the ability of the modified agent to modulate the activity of the LVCa(v) polypeptide in the non-excitable cell is measured by evaluating bulk calcium influx. The non-excitable cell can be, an immune system cell such as a T cell, B cell, or mast cell. The invention also includes a modified agent identified by any of the methods described herein.

Also featured is a method for identifying a candidate modulator of activity of an LVCa(v) polypeptide in a non-excitable cell. The method includes providing a non-excitable cell; contacting the cell with a test compound; measuring the ability of the test compound to inhibit calcineurin activity in the non-excitable cell; and testing the ability of the compound to inhibit bulk calcium influx in the non-excitable cell, such that, a compound that can inhibit calcineurin activity and bulk calcium influx in the non-excitable cell is a candidate modulator of the LVCa(v) polypeptide. The invention includes a modulator of an LVCa(v) polypeptide in a non-excitable cell identified by a method described herein.

The invention relates to a method for identifying a nucleic acid sequence that can inhibit expression of an LVCa(v) gene. The method includes transfecting a cell with an expression vector including a nucleotide sequence including at least 19 contiguous nucleotides of an LVCa(v) cDNA sequence; culturing the cell under conditions sufficient for expression of the nucleotide sequence, measuring the level of expression of the LVCa(v) mRNA or polypeptide in the cell, such that a decrease in the level of expression of the LVCa(v) mRNA or polypeptide indicates that the nucleic acid sequence can inhibit expression of the LVCa(v). In some embodiments, the cDNA is selected from the group consisting of a sequence depicted in any one of FIGS. 1A, 1C, 2A, 2C, 2E, 3A, 4A, 6A, 7, 8A, 9, 10A, 10C, 5C, 5D, 10E, degenerate variants thereof, or a complement thereof. In some cases, calcium influx is assayed in the cell and calcium influx is inhibited when the nucleic acid sequence is expressed.

Also featured is an RNAi agent derived from a nucleic acid sequence selected from the group consisting of a sequence depicted in any one of FIGS. 1A, 1C, 2A, 2C, 2E, 3A, 4A, 6A, 7, 8A, 9, 10A, 10C, 5C, 5D, 10E, degenerate variants thereof, or a complement thereof.

The invention also features a method for inhibiting expression of an LVCa(v) nucleic acid sequence. The method includes introducing an RNAi agent complementary to at least 19 contiguous nucleotides of the LVCa(v) nucleic acid sequence into a cell.

Also included is a method for inhibiting expression of an LVCa(v) gene in a subject in need thereof. The method includes administering a therapeutically effective amount of an RNAi agent targeted to an LVCa(v) nucleotide sequence to the subject. In some embodiments, the RNAi agent is an RNAi agent of identified using a method described herein.

The invention also relates to an antisense agent derived from a nucleic acid sequence depicted in any one of FIGS. 1A, 1C, 2A, 2C, 2E, 3A, 4A, 6A, 7, 8A, 9, 10A, 10C, 5C, 5D, 10E, degenerate variants thereof, or a complement thereof.

Also featured is a method for inhibiting expression of an LVCa(v) gene in a cell. The method includes introducing an antisense agent complementary to a portion of the nucleotide sequence of the LVCa(v) gene into the cell.

In addition, the invention relates to a method for inhibiting expression of an LVCa(v) gene in a subject in need thereof, the method including administering therapeutically effective amount of an antisense agent complementary to a portion of the LVCa(v) gene to the subject. The antisense agent can be an antisense agent identified using a method described herein.

The invention also features a calcium channel including an LVCa(v) polypeptide or variant thereof including one of more conservative substitutions, and when the LVCa(v) polypeptide or variant is expressed in a cell, the cell exhibits an L-type current having a reversal potential of about 0 mV and a peak amplitude of about 3-5 pA. In some embodiments, the I/V curve of the cell has the characteristics of FIG. 23. The calcium can be expressed in, e.g., a non-excitable cell and the LVCa(v) polypeptide can be a recombinant LVCa(v) polypeptide. Also featured is a calcium channel including an LVCa(v) polypeptide, such that activity of the calcium channel is modulated by phosphorylation of the LVCa(v) polypeptide. In some embodiments, the LVCa(v) polypeptide is an LVCa(v)1.3 polypeptide and activity is modulated by phosphorylation of the A/V exon of the LVCa(v)1.3 polypeptide.

Also included is a calcium channel that is expressed in a T cell, the calcium channel including a polypeptide, such that activity of the channel is modulated by phosphorylation of an N-terminus sequence of the polypeptide, e.g., an N-terminus sequence that is encoded by the first two exons of the mRNA encoding the polypeptide. In some cases, the calcium channel comprises an LVCa(v) polypeptide or variant thereof including one or more conservative amino acid substitutions, and the channel is modulated by phosphorylation of the A/B exon, e.g., the A/B exon is phosphorylated at the TSS site.

Also featured is a method of modulating calcium influx in a cell. The method includes contacting a cell with a compound that affects phosphorylation of an LVCa(v)1.3 polypeptide. In some cases, the cell is a non-excitable cell such as a T cell, mast cell, or B cell. In some embodiments, the compound affects phosphorylation of exon A/B of an LVCa(v)1.3 polypeptide.

The invention includes a method of modulating cell proliferation. The method includes contacting a cell with a compound that affects phosphorylation of an LVCa(v)1.3 polypeptide, for example, the compound affects phosphorylation of exon A/B of an LVCa(v)1.3 polypeptide.

In some cases, the invention relates to a method of inhibiting calcium influx into a non-excitable cell that expresses an LVCa(v) polypeptide. The method includes contacting the cell with a selective inhibitor of the LVCa(v) polypeptide, e.g., an LVCa(v)1.3 polypeptide.

The invention also relates to a method of identifying a subject having a proliferative cell disorder or who is at risk of developing a proliferative cell disorder. The method includes obtaining a sample from the subject; and determining whether the subject has an aberrant level of expression of an LVCa(1.3), such that an aberrant level of expression of an LVCa(1.3) compared to the level of expression in a normal population indicates that the subject has a proliferative cell disorder or is at risk for developing a proliferative cell disorder. In some embodiments, the level of expression of the LVCa(1.3) is elevated compared to a normal population. The proliferative cell disorder can be a disorder that includes undesirable proliferation of T cells. In some cases, the level of expression of the LVCa(1.3) is decreased compared to a normal population, for example, the proliferative cell disorder can be one in which there is an undesirably low level of T cell proliferation compared to a normal population.

An electrically non-excitable cell (non-excitable cell) is a cell that is not normally electrically excitable in that Ca2+ influx is not initiated by electrical activity at the plasma membrane of the cell. Examples of non-excitable cells are lymphocytes (e.g., T cells and B cells) and mast cells. Additional examples include other formed elements of blood, epithelial cells, connective tissue cells, and cell lines derived from any of the foregoing cell types.

A “Ca(v) polypeptide” is a calcium channel having the activity of one or more of the following types of calcium channels: Ca(v)1.1, Ca(v)1.2, Ca(v)1.3, Ca(v)1.4, Ca(v)2.2, Ca(v)2.3, Ca(v)3.1, Ca(v)3.2, Ca(v)3.3. A calcium channel having the activity of a Ca(v)2.1 channel is expressly excluded from the term “Ca(v) polypeptide” as used herein.

A “modulator of a Ca(v) polypeptide in a non-excitable cell” is an agent that preferentially modulates one or more of the active calcium influx processes in one or more types of non-excitable cells, without having a significant effect on calcium influx processes within excitable cells (e.g., cardiac, neuroendocrine or neural origin). Preferential modulation in this context means that the modulator has greater activity (e.g., modulation of calcium influx) against non-excitable versus excitable cells. Significant effect on calcium influx processes within excitable cells in this context means direct inhibition of Ca(v) polypeptides in excitable cells (e.g., measured using methods known in the art). For example, inhibiting a cardiac or neural cell at a level of less than about 20% compared to control, for example, less than about 15%, less than about 10%, or less than about 5%, represents a lack of significant effect.

Specific modulators of Ca(v) polypeptides can have the additional advantage of preferentially modulating Ca(v) polypeptides in one or more non-excitable cell types versus others (e.g., cells within the immune system). For example, particular modulators according to this invention may preferentially inhibit Ca(v) polypeptides in T cells versus B cells, mast cells, macrophages, or other non-excitable cell types. Without wishing to be bound by theory, the data provided herein demonstrate differential expression of Ca(v) genes within subsets of the immune system and differential expression is predicted to occur in other non-excitable cell types. The differential expression profiles of Ca(v) expression in non-excitable cells can be exploited to specifically tailor drug discovery, design, and assay development.

In addition to activities described above, a modulator of the expression or activity of an LVCa(v) polypeptide may affect one or more one or more functions of immune system cells that are modulated by calcium flux such as cell proliferation, cytokine synthesis, and production of mediators of tissue invasion.

Without wishing to be bound by theory, it is herein predicted that examples of existing structural classes of Ca(v) polypeptide inhibitors in excitable cells (e.g., dihydropyridines, phenylalkylamines and benzothiazapines) can be specifically altered in structure to preferentially inhibit Ca(v) polypeptides in non-excitable cells. This process will be facilitated using the sequences, methods, and assays of this invention.

A “non-voltage-gated selective inhibitor” (NV inhibitor) is a compound that preferentially inhibits non-voltage-gated (NV) Ca2+ influx in a cell compared to voltage-gated (VG) Ca2+ influx. In general, an NV inhibitor reduces NV Ca2+ influx by at least 50% compared to a control. In some embodiments, the NV Ca2+ influx is reduced by at least 60%, 70%, 80%, 90%, or 100% compared to a control. An NV inhibitor can reduce VG Ca2+ by not more than 0%, 5%, 10%, 30%, or 40%. In general, an NV inhibitor reduces NV Ca2+ influx by at least 50% and reduces VG Ca2+ influx by not more than 5%.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded.

The term “isolated or purified nucleic acid molecule” includes nucleic acid molecules that are separated from other nucleic acid molecules that are present in the natural source of the nucleic acid. For example, with regard to genomic DNA, the term “isolated” includes nucleic acid molecules that are separated from the chromosome with which the genomic DNA is naturally associated. In general, an “isolated” nucleic acid is free of sequences that naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in Current Protocols in Molecular Biology (John Wiley & Sons, N.Y., 1989, 6.3.1-6.3.6). Aqueous and non-aqueous methods are described in the art, and either can be used. An example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions is hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Generally, stringent hybridization conditions are hybridization in 6× sodium chloride/sodium citrate (SSC) at about 45° C., followed by one or more washes in 0.2×SSC, 0.1% SDS at 65° C. If the practitioner is uncertain about what conditions should be applied to determine if a molecule is within a hybridization limitation of the invention, the conditions can be 0.5 M sodium phosphate, 7% SDS at 65° C., followed by one or more washes at 0.2×SSC, 1% SDS at 65° C. In general, an isolated nucleic acid molecule of the invention hybridizes under stringent conditions to the sequence of an LVCa(v), or the complement thereof, and corresponds to a naturally occurring nucleic acid molecule.

The term “polypeptide” means any chain of amino acids, regardless of length or post-translational modification (e.g., glycosylation or phosphorylation), and includes natural proteins as well as synthetic or recombinant polypeptides and peptides.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of an LVCa(v) protein having less than about 30%, 20%, 10%, or generally 5% (by dry weight), of non-LVCa(v) protein (also referred to herein as a “contaminating protein”), or of chemical precursors or non-precursor chemicals. When the LVCa(v) protein or biologically active portion thereof is recombinantly produced, it is also generally substantially free of culture medium, i.e., culture medium represents less than about 20%, less than about 10%, or, generally less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of an LVCa(v) without abolishing or, in some cases, without substantially altering a biological activity, whereas an “essential” amino acid residue results in such a change.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an LVCa(v) protein is generally replaced with another amino acid residue from the same side chain family. Alternatively, in another embodiment, mutations can be introduced randomly along all or part of an LVCa(v) coding sequence, such as by saturation mutagenesis, and the resultant mutants can be screened for an LVCa(v) biological activity to identify mutants that retain activity.

Calculations of homology or sequence identity between sequences (the terms are used interchangeably herein) are performed as follows.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). Generally, the length of a reference sequence aligned for comparison purposes is at least, for example, 30%, at least 40%, at least 50%, at least 60%, or at least 70%, 80%, 90%, or 100% of the length of the reference sequence (e.g., when aligning a second sequence to the LVCa(v) sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. Generally, the percent identity between two amino acid sequences is determined using the Needleman and Wunsch (J. Mol. Biol. 48:444-453 (1970)) algorithm that has been incorporated into the GAP program in the GCG software package (available on the internet at gcg.com), using either a Blossum 62 matrix or a PAM250 matrix, and a gap weight of 16, 14, 12, 10, 8, 6, or 4 and a length weight of 1, 2, 3, 4, 5, or 6. In another embodiment, the percent identity between two nucleotide sequences is determined using the GAP program in the GCG software package (available on the internet at www.gcg.com), using a NWSgapdna.CMP matrix and a gap weight of 40, 50, 60, 70, or 80 and a length weight of 1, 2, 3, 4, 5, or 6. A set of parameters that should be used if the practitioner is uncertain about what parameters should be applied to determine if a molecule is within a sequence identity or homology limitation of the invention are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller (CABIOS, 1989, 4:11-17), which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul et al. (1990, J. Mol. Biol. 215:403-10). BLAST nucleotide searches can be performed with the NBLAST program, score=100, wordlength=12 to obtain nucleotide sequences homologous to LVCa(v) nucleic acid molecules of the invention. BLAST protein searches can be performed with the XBLAST program, score=50, wordlength=3 to obtain amino acid sequences homologous to LVCa(v) protein molecules of the invention. To obtain gapped alignments for comparison purposes, Gapped BLAST can be utilized as described in Altschul et al. (1997, Nucleic Acids Res. 25:3389-3402). When utilizing BLAST and Gapped BLAST programs, the default parameters of the respective programs (e.g., XBLAST and NBLAST) can be used. These programs are available on the Internet at ncbi.nlm.nih.gov.

A molecule (e.g., antibody) that “specifically binds” is one that binds to a particular polypeptide, e.g., an LVCa(v), but that does not substantially recognize or bind to other molecules in a sample, e.g., a biological sample that includes an LVCa(v).

References to constructs made of an antibody (or fragment thereof) coupled to a compound comprising a detectable marker include constructs made by any technique, including chemical means and recombinant techniques.

An animal, e.g., human, is “at risk” for developing a condition if there is an increased probability that they will develop the condition compared to a population (e.g., the general population, an age-matched population, a population of the same sex). The increased probability can be due to one or a combination of factors including the presence of specific alleles/mutations of a gene or exposure to a particular environment. For example, an individual is at risk for developing an autoimmune disorder when they exhibit an aberrant level of an LVCa(v) protein compared to a control population.

The amount of expression of activity of an LVCa(v) in a test cell (e.g., a lymphocyte from an individual having a lymphocytic disorder) can be evaluated by comparing it to a predetermined (reference) value, e.g., the level of expression in a normal lymphocyte, e.g., a T cell or B cell.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### DESCRIPTION OF DRAWINGS

FIG. 1A is a representation of the nucleic acid sequence of exon A.

FIG. 1B is a representation of the predicted amino acid sequence of exon A.

FIG. 1C is a representation of the nucleic acid sequence of exons B.

FIG. 1D is a representation of the predicted amino acid sequence of exon B.

FIG. 2A is a representation of the nucleic acid sequence of exon 33a.

FIG. 2B is a representation of the predicted amino acid sequence of exon 33a.

FIG. 2C is a representation of the nucleic acid sequence of exon 32-34 (including exon 33a).

FIG. 2D is a representation of the predicted amino acid sequence of exons 32-34 (including exon 33a).

FIG. 2E is a representation of the nucleic acid sequence of exons 32-34 without exons 33 or 33a.

FIG. 2F is a representation of the predicted amino acid sequence of exons 32-34 without exons 33 or 33a.

FIG. 3A is a representation of the nucleic acid sequence of exons 11-13.

FIG. 3B is a representation of the predicted amino acid sequence of exons 11-13.

FIG. 4A is a representation of the nucleic acid sequence of exon A/B.

FIG. 4B is a representation of the predicted amino acid sequence of exon A/B.

FIG. 5A is a representation of a nucleic acid sequence of exon 44.

FIG. 5B is a representation of the predicted amino acid sequence of exon 44.

FIG. 5C is an alignment showing 3′ untranslated sequence of LVCa(v)1.3.

FIG. 5D is an alignment showing 5′ untranslated sequence of LVCa(v)1.3.

FIGS. 6A-6C are a representation of a nucleic acid sequence of the coding region of LV1Ca(v)1.3.

FIG. 6D is a representation of a predicted amino acid sequence of LV1Ca(v)1.3.

FIGS. 7A-7C are a representation of a full-length nucleic acid sequence of LV1Ca(v)1.3, including 3‘and S’ untranslated regions.

FIGS. 8A-8C are a representation of the nucleic acid sequence of the coding region of LV2Ca(v)1.3.

FIG. 8D is a representation of the predicted nucleic acid sequence of the coding region of LV2Ca(v)1.3.

FIGS. 9A-9C are a representation of a full-length nucleic acid sequence of LV2Ca(v)1.3 including 3‘and S’ untranslated regions.

FIG. 10A is a representation of the nucleic acid sequence of exon A′.

FIG. 10B is a representation of the predicted amino acid sequence of exon A′.

FIG. 10C is a representation of the nucleic acid sequence of exon B′.

FIG. 10D is a representation of the predicted amino acid sequence of exon B′.

FIG. 10E is a representation of an alignment of the 5′ noncoding sequence of LVCa(v)1.1.

FIG. 11 is a schematic representation of the cloning strategy for LVCa(v)1.3.

FIG. 12 depicts a comparison between the amino acid sequence of the first exon of Ca(v)1.3 and exon A/B (alt exon 1) of LV1-Ca(v)1.3.

FIGS. 13A-13C are a representation of the nucleic acid sequence of a three-domain variant of Ca(v)1.3.

FIG. 14 is a representation of the amino acid sequence of the three-domain variant of Ca(v)1.3.

FIG. 15 is a representation of the amino acid sequence of exon 50 of Ca(v)1.3.

FIG. 16 is a set of recordings depicting calcium influx in cells containing an expressed three-domain Ca(v)1.3 and in cells containing wild type Ca(v)1.3 only.

FIG. 17 is a representation of a sequence comparison between chicken and human exon 5 of Ca(v)1.3.

FIG. 18 is a schematic drawing of the cloning strategy for a knockout of Ca(v)1.3 sequence in DT40 cells.

FIG. 19 is a set of recordings depicting the results of an experiment measuring calcium influx in wild type cells and cells knocked out for a Ca(v)1.3 allele.

FIGS. 20A-20C are bar graphs depicting the results of experiments in which expression of specific L-type channel proteins was assayed using quantitative PCR.

FIG. 21 is a set of recordings depicting the results of an experiment measuring calcium influx in wild type Jurkat cells and Jurkat cells knocked down for L-type channel sequences by siRNA.

FIG. 22A is a set of flow cytometry traces illustrating the results of experiments in which cells from various cell lines (as labeled for each panel) were untreated (un), stained with DM-BODIPY (DM-BODIPY), or treated with BayK8644 and stained with DM-BODIPY (+BayK).

FIG. 22B is a reproduction of an image of a stained gel containing Ca(v)1.3 PCR products amplified from various cell lines.

FIG. 23 is a representation of an I/V curve of a Jurkat cell using calcium as the carrier (cntrl) and a Jurkat cell incubated in the presence of BayK8644 using calcium as the carrier.

FIG. 24A is a reproduction of an image of a stained gel showing the results of PCR experiments detecting the presence of Ca(v)1.3 expression in cells transfected with empty vector, vector expressing a scrambled siRNA, or expressing an siRNA targeting Ca(v)1.3 (D1, D2, D3). β-Actin was included as a control.

FIG. 24B is a bar graph illustrating the results of experiments in which the relative growth of cells transfected as described for FIG. 24A was determined using an MTT assay.

FIG. 24C is a bar graph illustrating the results of experiments in which the relative growth of cells transfected as described for FIG. 24A was determined using a BrdU assay.

FIG. 25A is a reproduction of a flow cytometry trace illustrating the results of experiments in which control wild-type (WT) and L-type siRNA-expressing (LT1) Jurkat T cells were stained with the DM-BODIPY dye.

FIG. 25B is a graphical representation of the average time course development of Icrac in WT (n=7) and siRNA-LT2 (n=5) Jurkat cells.

FIG. 25C is a pair of reproductions of raw data traces of an example WT cell (upper panel) and an siRNA-LT2 cell at 100 seconds into a whole-cell experiment.

FIG. 25D is a representation of a current amplitude histogram of siRNA-LT1 (n=20) and siRNA-LT2 (n=5) in comparison to current amplitude histogram of WT cells (n=27).

FIG. 26 is a reproduction of an image of a stained gel containing PCR products resulting from amplification of an LVCa(v)1.3 sequence in cells from a blood sample (B/T/monocytes), CD4(+) T cells, and CD8(+) T cells. Markers (M).

FIG. 27A is a reproduction of an image of a stained gel showing expression of Flag-LVCav1.3 expression in three independent cell lines (1, 2, and 3) of Jurkat cells that were induced (+) or uninduced (−) with doxycycline.

FIG. 27B is a bar graph depicting the results of experiments in which cells that were stably transfected with Flag-LVCav1.3, induced (+) or uninduced (−) with doxycycline, and the relative cell growth assayed.

### DETAILED DESCRIPTION

Calcium channels that are specifically or preferentially expressed in a specific cell type provide excellent targets for identifying and developing compounds that can affect processes within the specific cell type while having a minimal effect on other types of cells having calcium channels. Because of the importance of calcium channels in regulation of cellular processes, identification of specifically expressed channels also facilitates methods of elucidating cell-specific processes and disorders associated with aberrant calcium signaling. The present invention relates to the discovery that cell-specific calcium channel proteins are expressed in non-excitable cells such as kidney cells, lymphocytes (e.g., T cells, B cells), and mast cells. These channel proteins are the result of translation alternative of spliced L-type channel subunit sequences, and the polypeptides resulting from translation of these alternatively spliced mRNAs are involved in regulating calcium influx in cells. Thus, the L-type channel nucleic acid molecules and polypeptides described herein are useful, e.g., as targets for identifying compounds that can modulate calcium flux in cells expressing these channels, for example, non-excitable cells. Compounds that can modulate calcium influx can be used to affect cellular processes associated with calcium flux, e.g., cellular proliferation.

The L-type calcium channel nucleic acid molecules and proteins that are expressed in non-excitable cells and are described herein are termed “LVCa(v).” These include portions of LVCa(v) nucleic acid sequences or polypeptide sequences that are not expressed to a significant extent in known Ca(v) sequences (e.g., neuronal Ca(v) polypeptides), and full-length sequences that include the such portions. Since LVCa(v) mRNAs are splice variants of Ca(v) sequences, certain portions of some LVCa(v) sequences are transcribed from known Ca(v) sequences. Therefore, references to numbered exons herein refer to the numbering of known exons for the specific Ca(v) gene that is referenced, unless otherwise indicated.

Novel LVCa(v) Nucleic Acid Sequences and Polypeptide Sequences

Nucleic acid molecules and polypeptides have been discovered that are expressed in proteins derived from an L-type calcium channel gene. The novel sequences encompassed by certain embodiments include novel exons and novel sequences created by splicing of a nucleic acid sequence as well as polypeptide sequences resulting from translation of the novel nucleic acid sequences.

Nucleic acid molecules described herein that do not correspond to a complete LVCa(v)-encoding sequence are useful, e.g., for specifically detecting expression of an LVCa(v) mRNA.

Novel polypeptide molecules that do not correspond to a full-length LVCa(v) are useful, e.g., for generating antibodies that specifically recognize an LVCa(v) polypeptide.

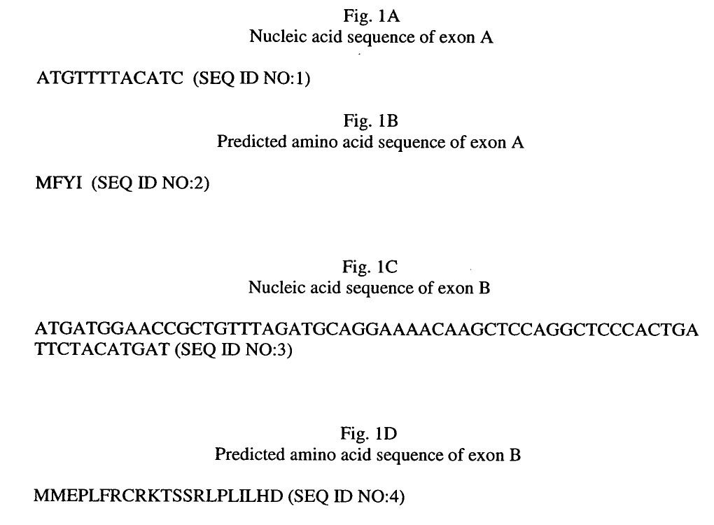

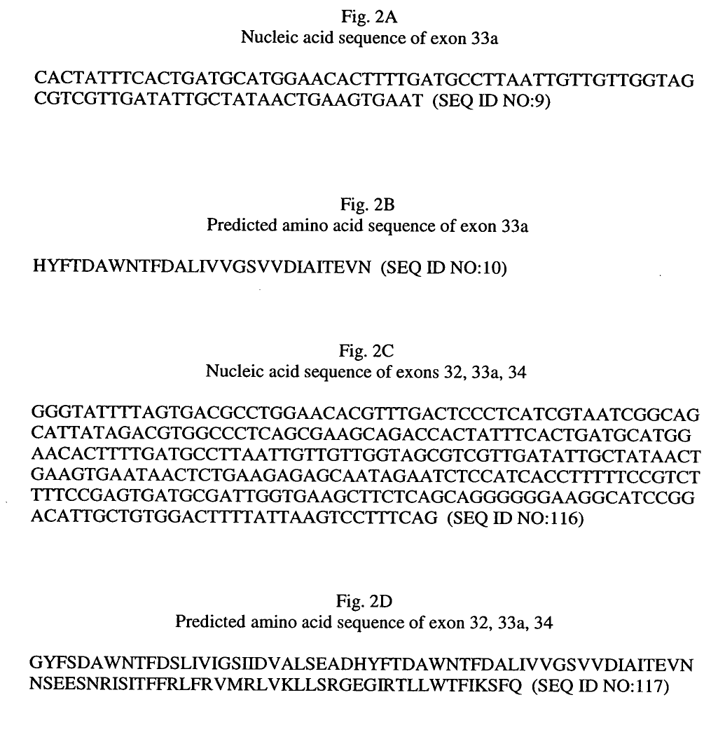

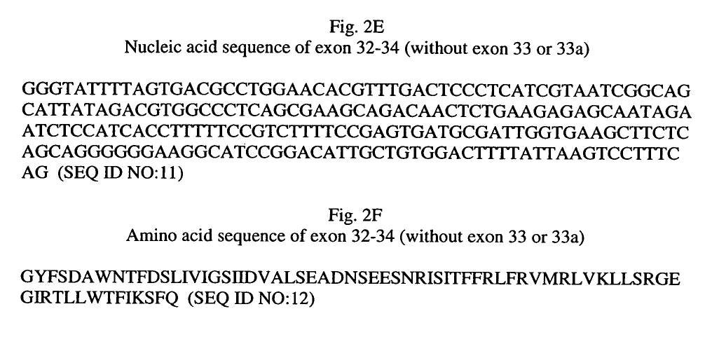

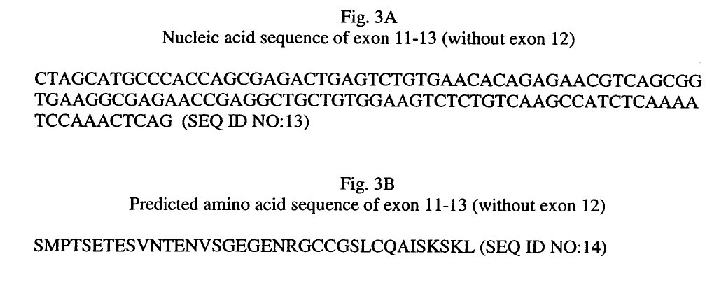

LVCa(v)1.3 nucleic acid sequences include sequences corresponding to variants of Ca(v)1.3-related sequences that contain novel exons and to Ca(v)1.3-related sequences created by the removal of exons previously described as part of Ca(v)1.3 sequences, as well as full-length sequence, e.g., containing coding sequence for LVCa(b)1.3. Included among these sequences are those termed exon A (SEQ ID NO:1, FIG. 1A) and exon B (SEQ ID NO:3; FIG. 1C), novel exon 33 (termed herein exon 33a; SEQ ID NO:9, FIG. 2A), a sequence created by the absence of exon 33 and thus conjoining exons 32 and 34 (termed herein exon 32-34; SEQ ID NO:11, FIG. 2E), a sequence created by the absence of exon 12 and thus conjoining exons 11 and 13 (termed exon 11-13, SEQ ID NO:13, FIG. 3A). The predicted amino acid sequences corresponding to these nucleic acid sequences are shown in FIGS. 1B (exon A, SEQ ID NO:2), 1D (exon B, SEQ ID NO:4), 2B (exon 33a, SEQ ID NO:10), 2F (exon 32-34, SEQ ID NO:12), and 3B (exon 11-13, SEQ ID NO:14).

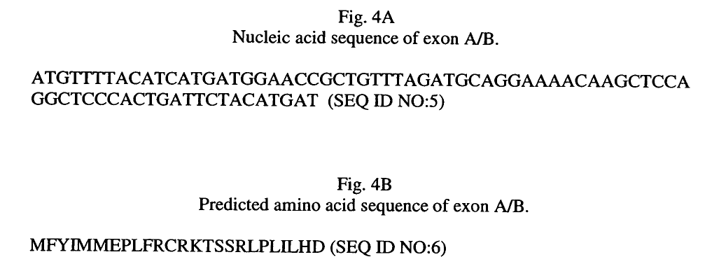

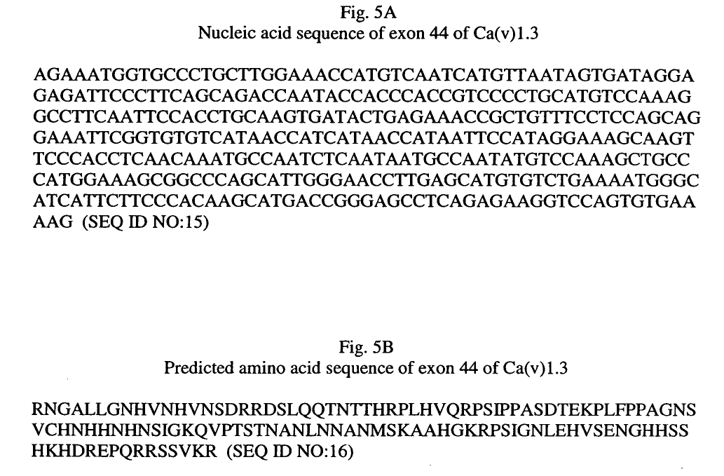

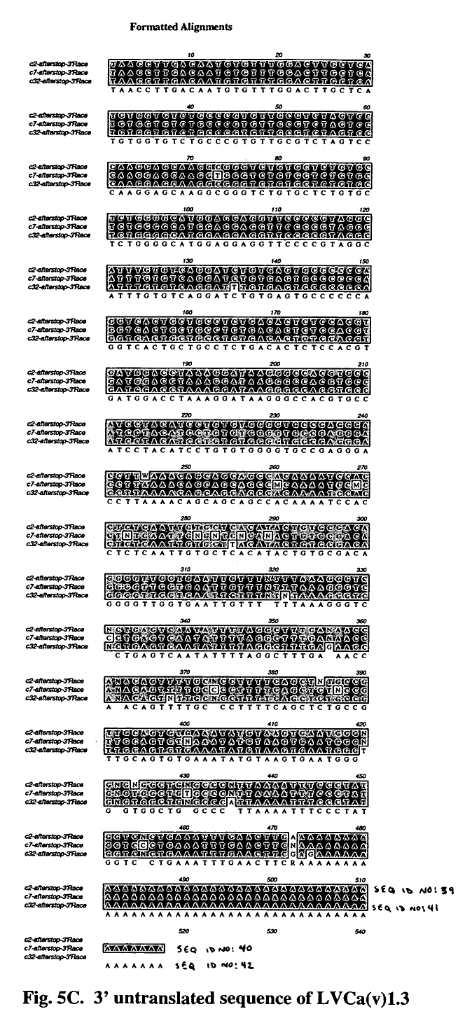

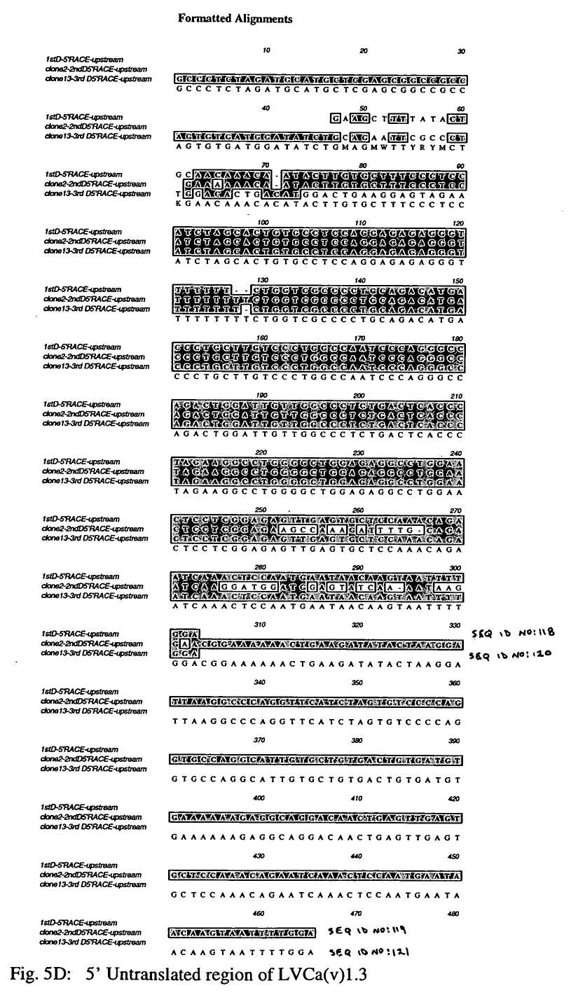

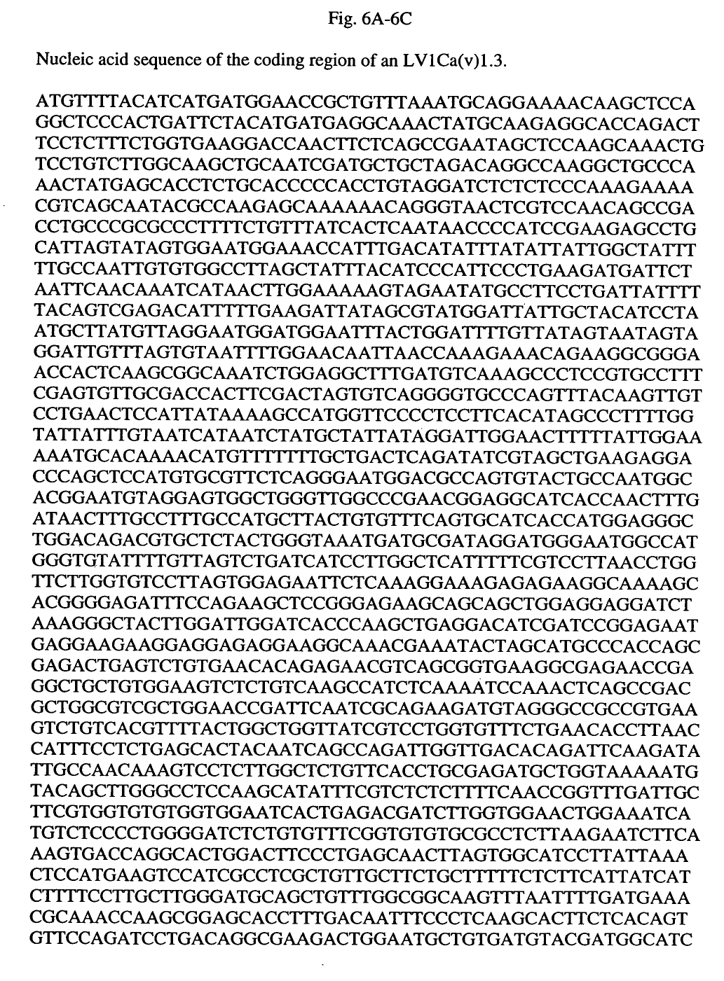

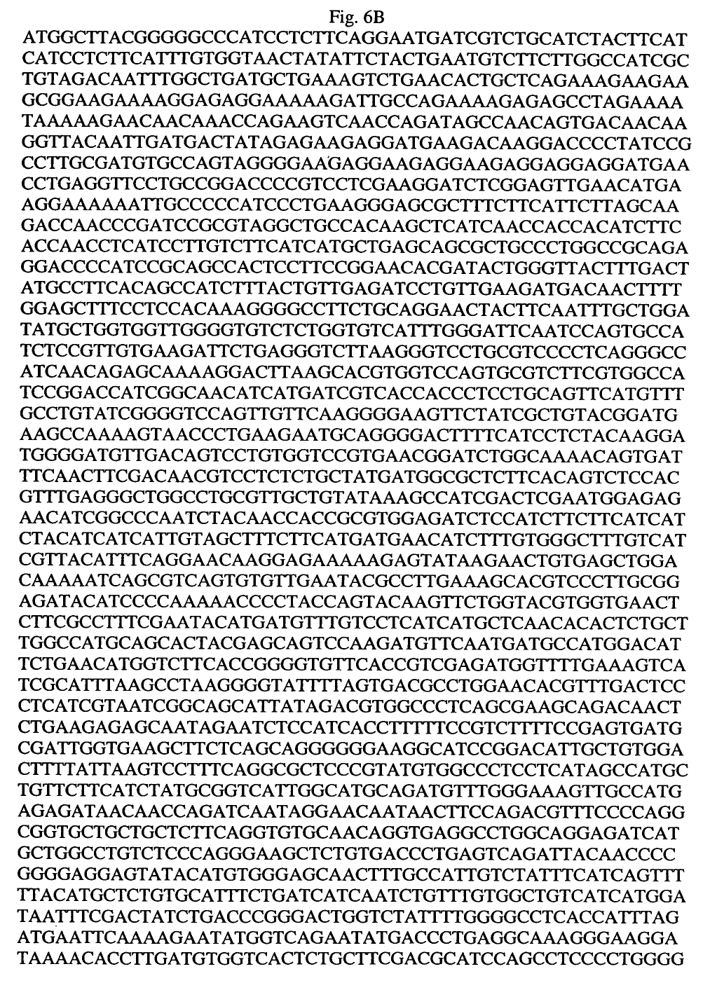

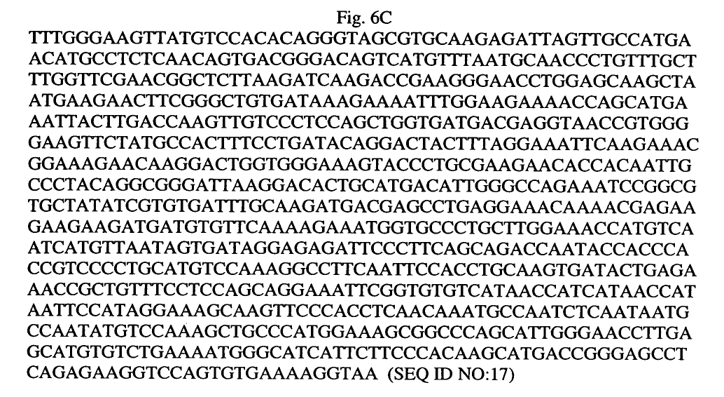

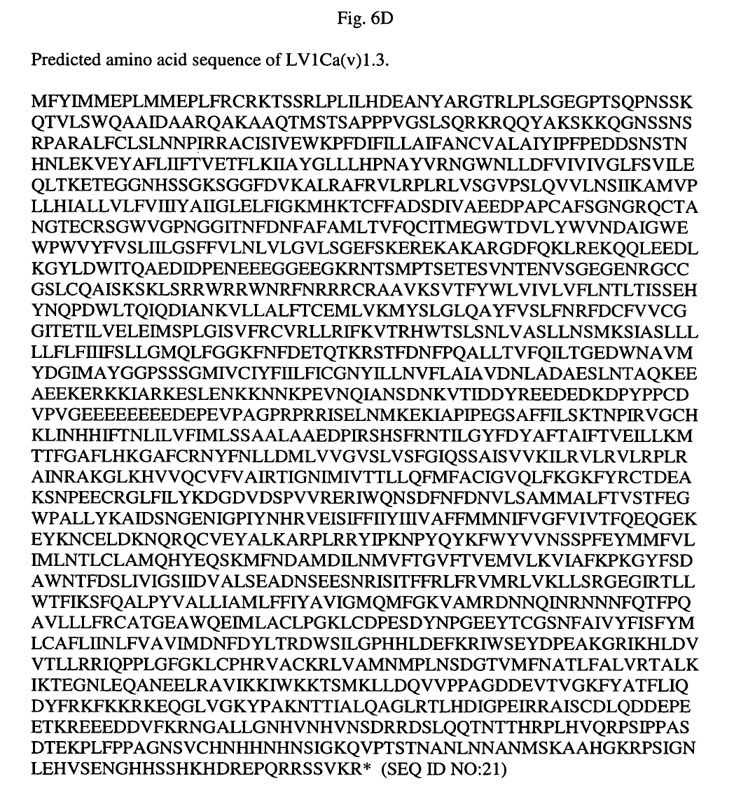

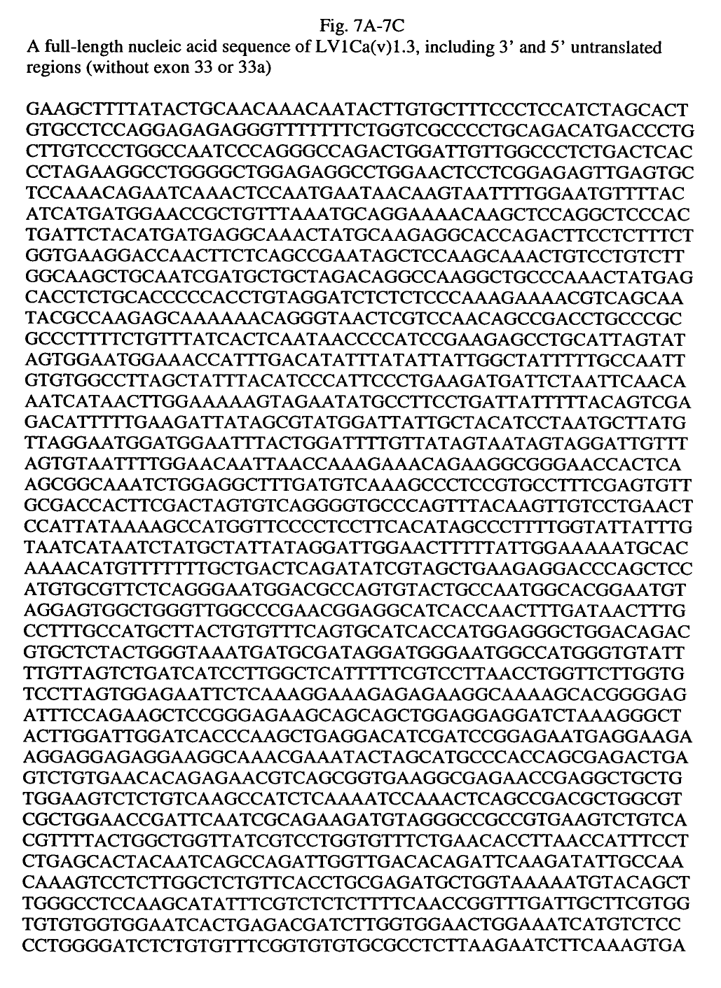

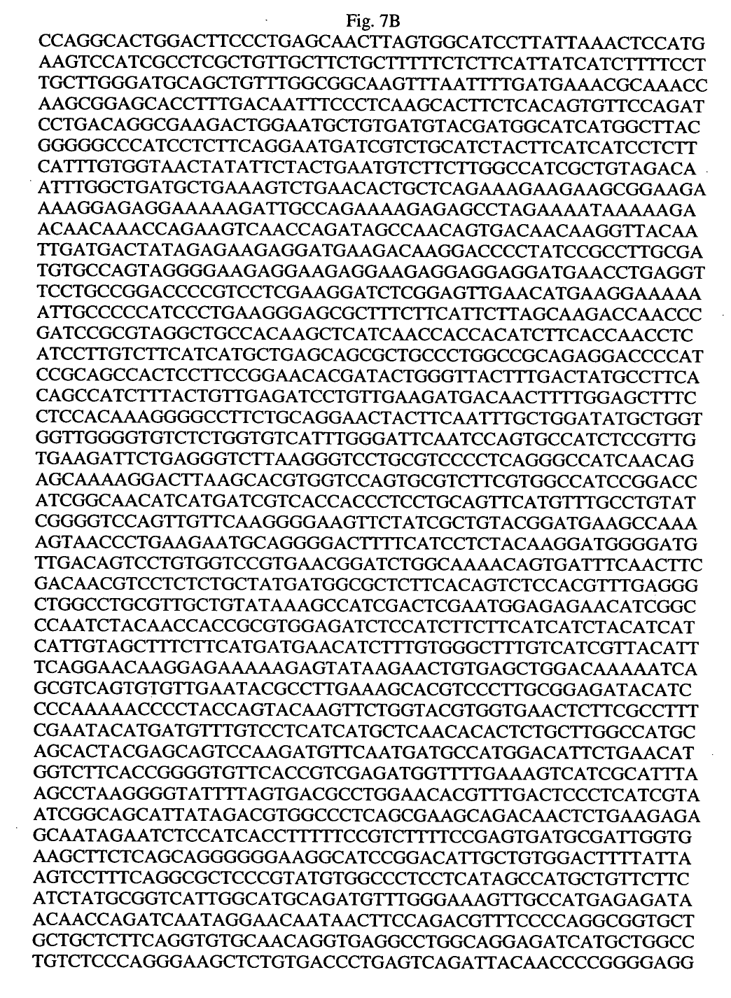

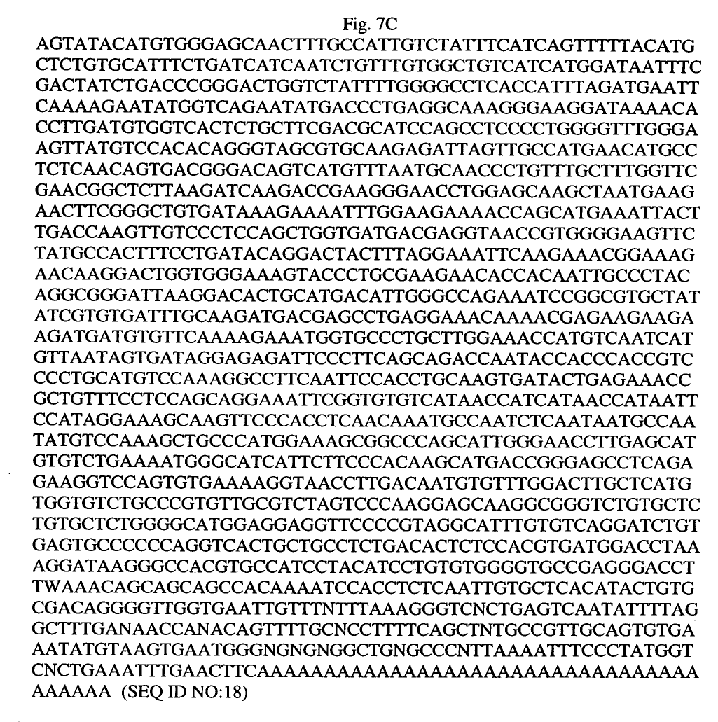

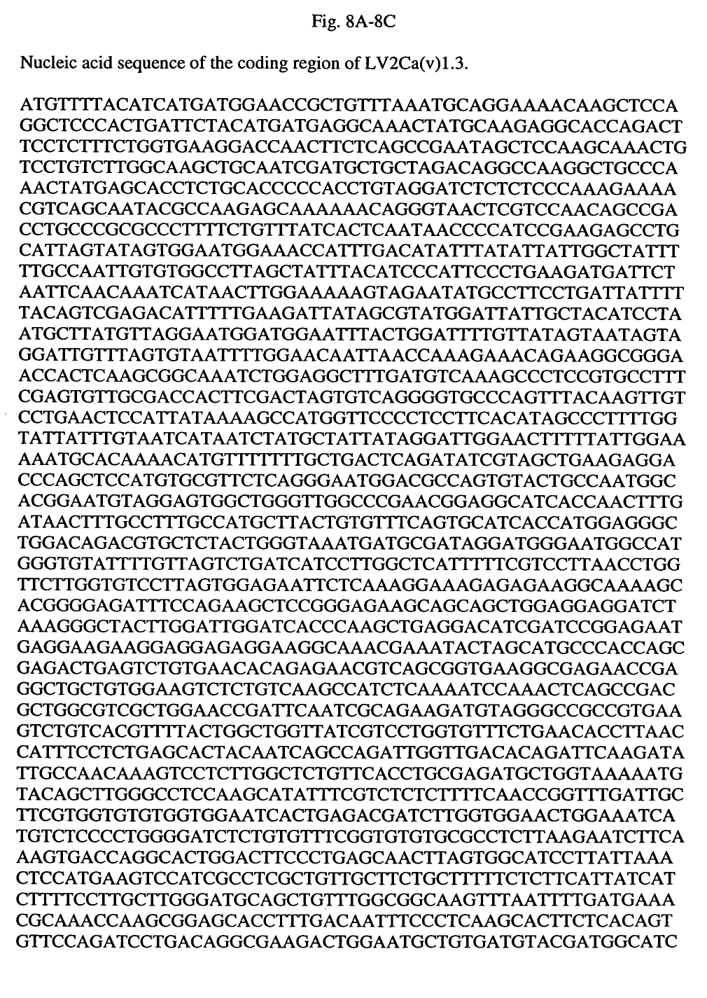

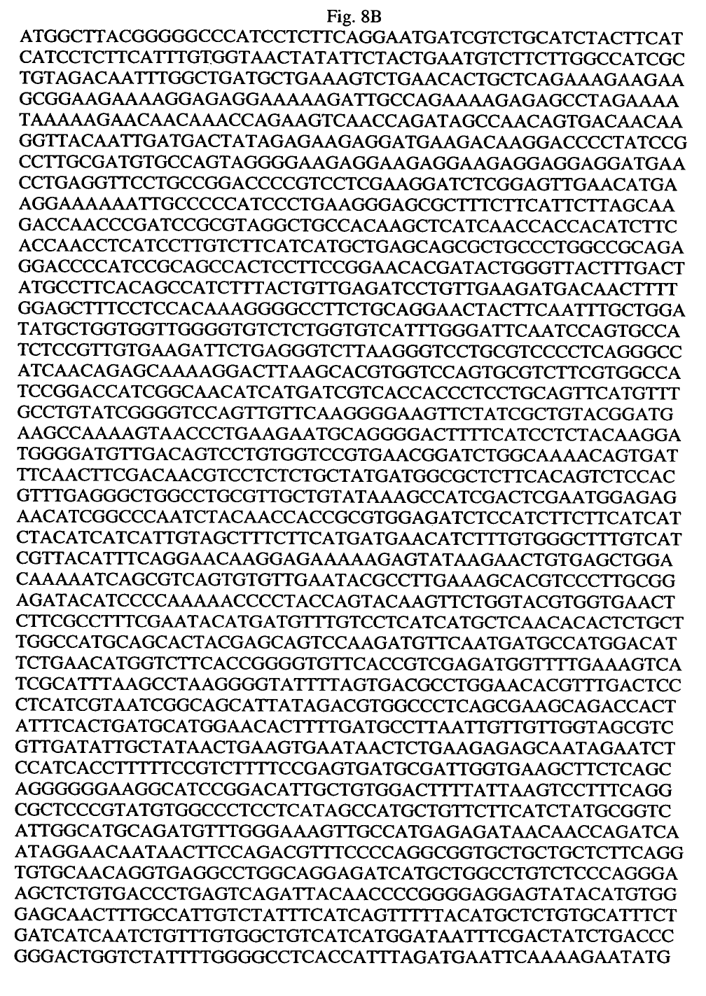

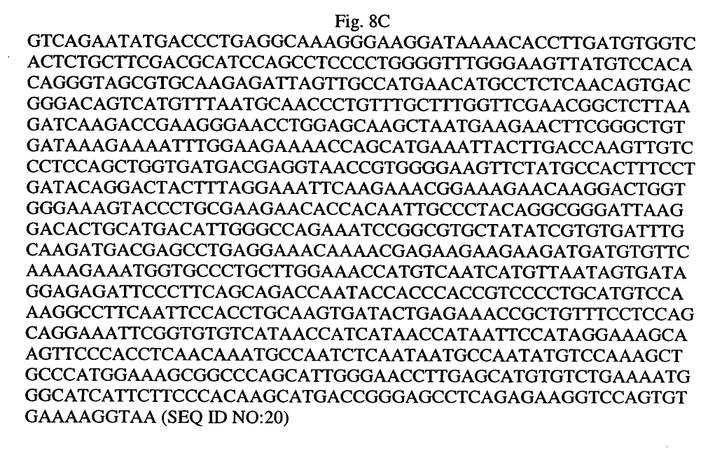

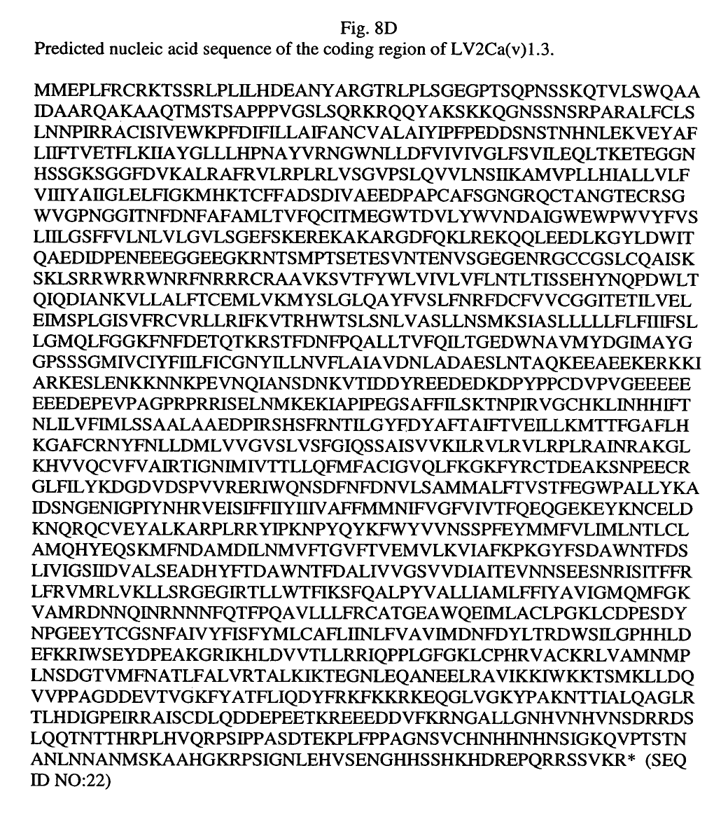

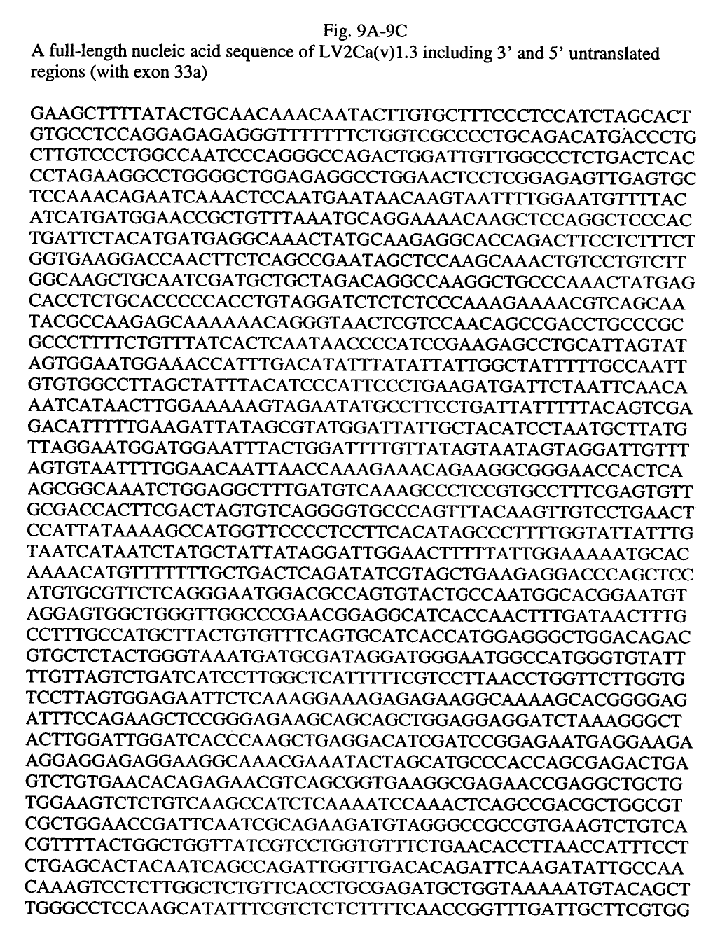

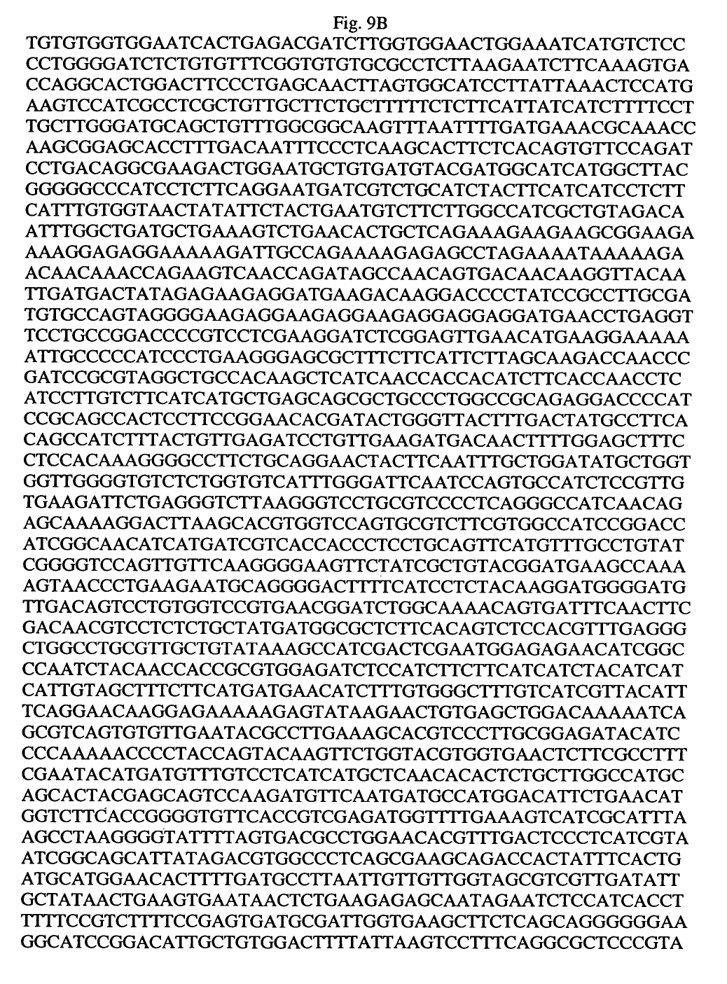

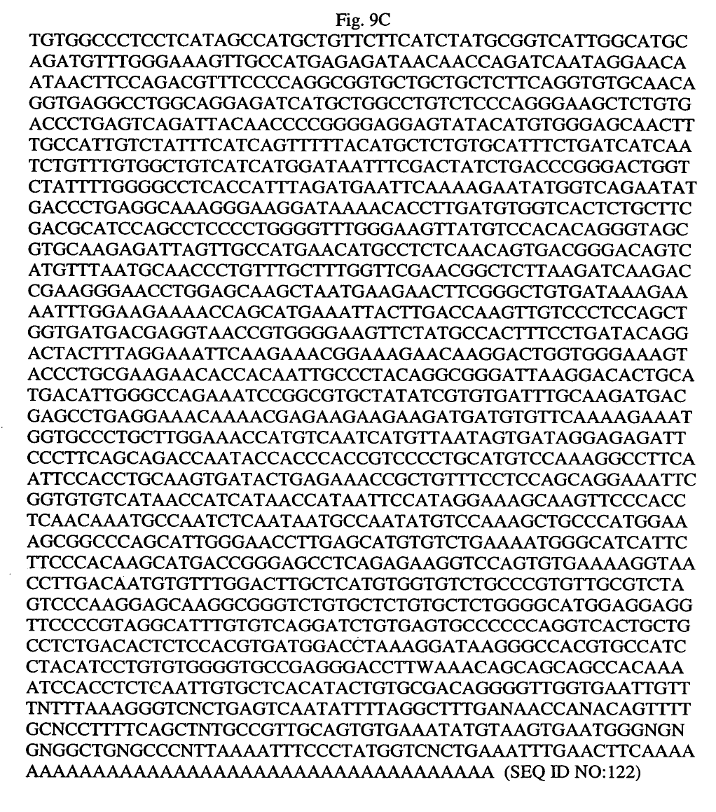

LVCa(v)1.3 (α1D)/Jurkat mRNA is an approximately 5.5 kb transcript that encodes a protein of about 5.5 kDa. The transcript encodes a novel sequence comprising novel exons A and B, which replace the previously described exon 1 (i.e., neuronal Ca(v)1.3 exon 1). The novel exon A/B nucleic acid sequence (SEQ ID NO:5) and predicted amino acid sequence (SEQ ID NO: 6) are shown in FIGS. 4A and 4B. In some embodiments, the LVCa(v)1.3 terminates at the end of exon 44 (FIGS. 5A and 5B; SEQ ID NOs:15 and 16). One LVCa(v)1.3 channel mRNA (LV1Ca(v)1.3) contains exons A/B, includes exon 12, contains exon 33a, and terminates after exon 44. The coding region of this transcript is shown in FIG. 6A-6C (SEQ ID NO:17) and a predicted amino acid sequence is shown in FIG. 6D (SEQ ID NO:21). In some cases, the predicted amino acid sequence has different start site as discussed below. An example of a full-length transcript for LV1Ca(v)1.3 is shown in FIG. 7 (SEQ ID NO:18). In other embodiments, the transcript encodes an exon A/B, includes exon 12, does not contain an exon 33, and terminates after exon 44. This sequence is referred to as LV2Ca(v)1.3. The nucleic acid sequence of LV2Ca(v)1.3 (coding region) is shown in FIGS. 8A-8C and the predicted translation is shown in FIG. 8D. An example of a full-length transcript of LV2Ca(v)1.3 is shown in FIGS. 9A-9C.

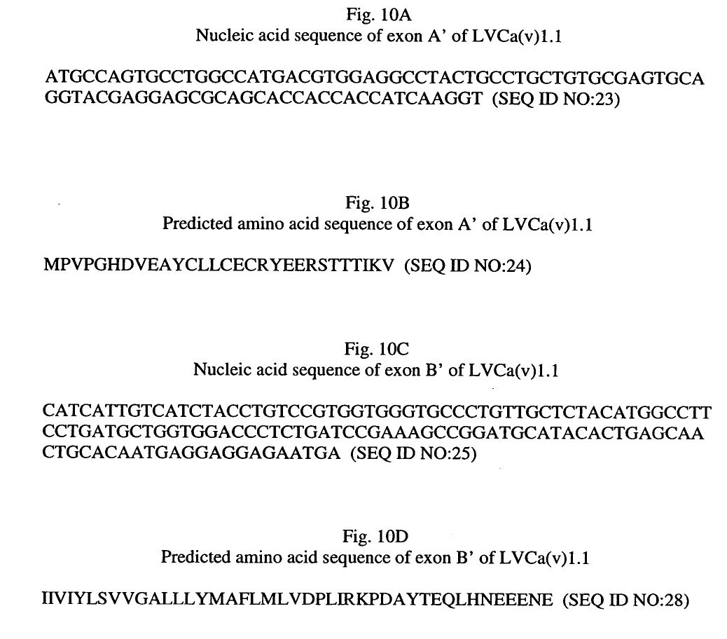

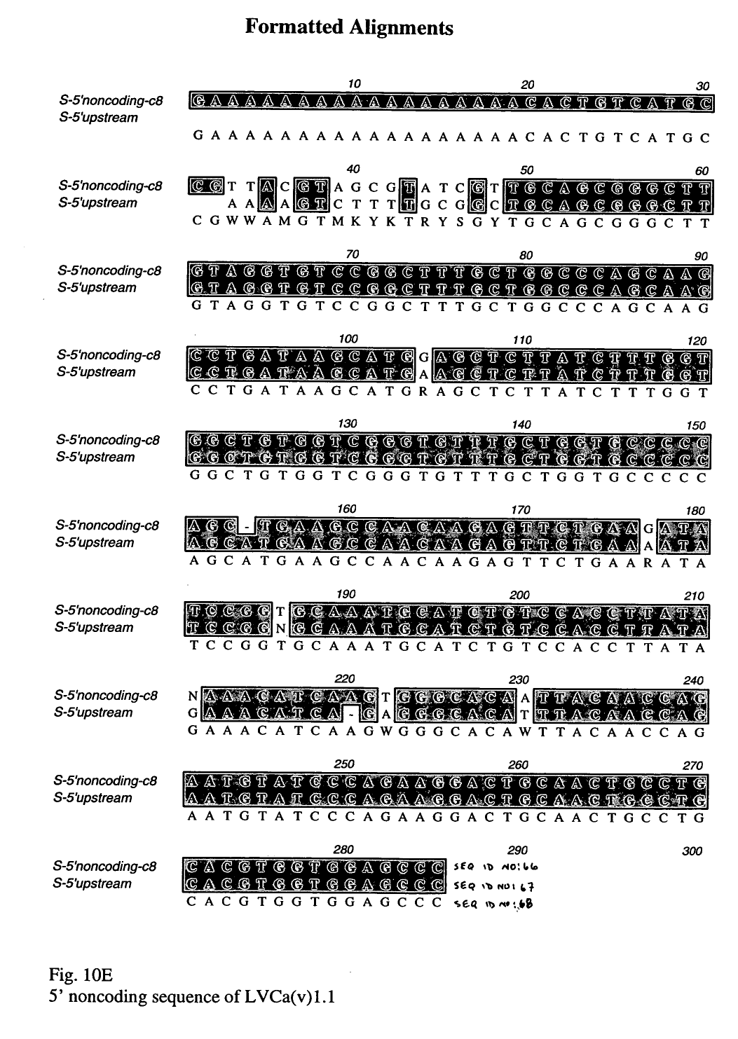

The invention also relates to novel Ca(v)1.1 nucleic acid sequences, termed LVCa(v)1.1 sequences. These include exons A′ and B′, which are shown in FIGS. 10A and 10C, respectively (SEQ ID NOs:23 and 25). These sequences are predicted to encode the amino acid sequences of exons A′ and B′ shown in FIGS. 10B and 10D, respectively (SEQ ID NOs:24 and 28). The LVCa(v)1.1 sequences lack the previously known exons 1-2 of Ca(v)1.1, which are replaced by exons A′ and B′. The combined sequences for exons A′ and B′ are termed herein exon A′/B′.

Isolated Nucleic Acid Molecules

Various methods of the invention employ an isolated or purified, nucleic acid molecule that encodes an LVCa(v) polypeptide, e.g., a full-length LVCa(v) protein or a fragment thereof, e.g., a biologically active portion of LVCa(v) polypeptide, as well as nucleic acid molecules that hybridize, e.g., under highly stringent conditions, to a nucleic acid molecule that encodes an LVCa(v) polypeptide and nucleic acid molecules having a defined degree of sequence identity (e.g., at least about 60%, 65%, 70%, 75%, 80%, 85%, 90%, 91%, 92%, 93%, 94%, 95%, 96%, 97%, 98%, 99% identity), to a nucleic acid molecule encoding an LVCa(v) polypeptide.

LVCa(v) probes and primers are useful in, for example, detection methods that require the detection of LVCa(v) expression. Typically a probe/primer is an isolated or purified oligonucleotide. The oligonucleotide typically includes a region of nucleotide sequence that hybridizes under stringent conditions to at least about 7, 12, 15, 20, 25, 30, 35, 40, 45, 50, 55, 60, 65, or 75 consecutive nucleotides of a sense or antisense sequence of an LVCa(v) nucleic acid molecule or of a naturally occurring allelic variant or mutant of an LVCa(v) nucleic acid molecule.

Primers suitable for use in a PCR, which can be used to amplify a selected region of an LVCa(v) sequence, are useful in certain methods of the invention. The primers should be at least 5, 10, or 50 base pairs in length and less than 100, or less than 200, base pairs in length.

Other useful nucleic acid molecules are greater than 260, 300, 400, 500, 600, 700, 800, 900, 1000, or 1100 or more nucleotides in length and hybridize under stringent hybridization conditions to an LVCa(v) nucleic acid molecule.

Nucleic acid molecules comprising or consisting of 100, 200, 300, 400, 500, 600, 700, 800, 900, 1000, or 1100 or more contiguous nucleotides of an LVCa(v) nucleic acid molecule are also useful in the methods of the invention.

Also useful in the methods of the invention nucleic acid molecules that differ from the nucleotide sequence of an LVCa(v) nucleic acid molecule that is described herein, but still encode the amino acid sequence of a corresponding LVCa(v). Other useful nucleic acid molecules encode a protein having an amino acid sequence that differs, by at least 1, but less than 5, 10, 20, 50, or 100 amino acid residues of an LVCa(v) polypeptide such as those disclosed herein. Other useful variants can be naturally occurring, such as allelic variants (same locus), homologs (different locus), and orthologs (different organism), or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce conservative, non-conservative, or both types of amino acid substitutions (compared to the encoded product). In general, the variations produce conservative amino acid substitutions.

Nucleic acids that encode allelic variants of LVCa(v) polypeptides include are also useful. Such nucleic acids can encode either functional or non-functional proteins. Functional allelic variants include naturally occurring amino acid sequence variants of the LVCa(v) protein within a population that maintain the ability to mediate at least one LVCa(v) biological activity. Functional allelic variants will typically contain only conservative substitutions of one or more amino acids of an LVCa(v) polypeptide, or substitution, deletion or insertion of non-critical residues in non-critical regions of the protein. Non-functional allelic variants are naturally occurring amino acid sequence variants of the LVCa(v) protein within a population that do not have the ability to mediate any LVCa(v) biological activity. Non-functional allelic variants will typically contain a non-conservative substitution, a deletion, or insertion, or premature truncation of the amino acid sequence of an LVCa(v) polypeptide, or a substitution, insertion, or deletion in critical residues or critical regions of the protein. Nucleic acids encoding such non-functional allelic variants are useful, e.g., for knocking out expression of an LVCa(v) nucleic acid sequence or polypeptide.

Certain mRNA molecules encoding the novel calcium channels described herein can be identified by their termini, which are at the end of the sequence encoded by exon 44. Similarly cDNAs derived from these molecules can be identified by such sequences. For example, certain LVCa(v) RNAs include sequence coding for exon 44 but not for exon 45 and so can be identified by their length or by their ability to hybridize exon 44 cDNA but not to cDNA for a subsequent exonic sequence (e.g., exon 45).

It is to be understood that complements of nucleic acid molecules described herein and double-stranded nucleic acid molecules can be useful, e.g., for cloning. In addition, one in the art would know that nucleic acid molecules can be ribonucleic acid molecules, deoxyribonucleic acid molecules, or certain synthetic nucleotides, and would know how to select the appropriate nucleic acid molecule using what is known in the art and the guidance provided herein.

Antisense Nucleic Acid Molecules, Ribozymes, siRNAs, and Modified LVCa(v) Nucleic Acid Molecules

Isolated nucleic acid molecules that are antisense to an LVCa(v) nucleotide sequence are useful for reducing activity or expression of the LVCa(v) mRNA or polypeptide. An “antisense” nucleic acid (antisense oligonucleotide or ASO) can include a nucleotide sequence that is complementary to a “sense” nucleic acid encoding a protein, e.g., complementary to the coding strand of a double-stranded cDNA molecule, or complementary to an mRNA sequence. The antisense nucleic acid can be complementary to an entire LVCa(v) coding strand, or to only a portion thereof (e.g., coding region of a human LVCa(v) nucleotide sequence such as the region encoding exon A, exon B, exon A′, or exon B′). In another embodiment, the antisense nucleic acid molecule is antisense to a “noncoding region” of the coding strand of a nucleotide sequence encoding an LVCa(v) polypeptide (e.g., the 5′ or 3′ untranslated regions).

An antisense nucleic acid can be designed such that it is complementary to the entire coding region of LVCa(v) mRNA, but in general, is an oligonucleotide that is antisense to only a portion of the coding or noncoding region of LVCa(v) mRNA. For example, the antisense oligonucleotide can be complementary to the region surrounding the translation start site of LVCa(v) mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules are typically administered to a subject (e.g., by direct injection at a tissue site), or generated in situ such that they hybridize with or bind to cellular RNA (e.g., mRNA) and/or genomic DNA encoding an LVCa(v) protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are generally used. Methods of administering antisense nucleic molecules are also known in the art (e.g., Wacheck et al., 2000, Lancet 356:1728-1733; Webb et al., 1997, Lancet 349:1137-1141; Vitranene2, Isis Pharmaceuticals, Inc.

An antisense nucleic acid can be an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids. Res. 15:6625-6641). The antisense nucleic acid molecule can also comprise a 2′-O-methylribonucleotide (Inoue et al., 1987, Nucleic Acids Res. 15:6131-6148) or a chimeric RNA-DNA analogue (Inoue et al., 1987, FEBS Lett. 215:327-330).

An antisense nucleic acid can also be a ribozyme. A ribozyme having specificity for a LVCa(v)-encoding nucleic acid can include one or more sequences complementary to the nucleotide sequence of an LVCa(v) cDNA disclosed herein (e.g., SEQ ID NO:17, SEQ ID NO:18, SEQ ID NO:122, or SEQ ID NO:20), and a sequence having known catalytic sequence responsible for mRNA cleavage (see, for example, U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in an LVCa(v)-encoding mRNA (see, e.g., Cech et al. U.S. Pat. No. 4,987,071; and Cech et al. U.S. Pat. No. 5,116,742). Alternatively, LVCa(v) mRNA can be used to select a catalytic RNA having a specific ribonuclease activity from a pool of RNA molecules (see, e.g., Bartel and Szostak (1993) Science 261:1411-1418).

LVCa(v) gene expression can be inhibited by targeting nucleotide sequences complementary to the regulatory region of the LVCa(v) (e.g., the LVCa(v) promoter and/or enhancers) to form triple helical structures that prevent transcription of the LVCa(v) gene in target cells (see generally, Helene (1991) Anticancer Drug Des. 6(6):569-84; Helene et al. (1992) Ann. N.Y. Acad. Sci. 660:27-36; and Maher (1992) Bioassays 14(12):807-15). The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Detectably labeled oligonucleotide primer and probe molecules are useful in the methods of the invention, e.g., diagnostic methods. Typically, such labels are chemiluminescent, fluorescent, radioactive, or colorimetric.

An LVCa(v) nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) Bioorganic & Medicinal Chemistry 4 (1): 5-23). As used herein, the terms “peptide nucleic acid” or “PNA” refer to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al. (1996) supra and Perry-O'Keefe et al. (1996, Proc. Natl. Acad. Sci. 93: 14670-14675).

PNAs of LVCa(v) nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of LVCa(v) nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup, 1996 supra)); or as probes or primers for DNA sequencing or hybridization (Hyrup et al., 1996, supra; Perry-O'Keefe et al., 1996, supra).

The oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al., 1988, Bio-Techniques 6:958-976) or intercalating agents. (e.g., Zon, 1988, Pharm. Res. 5:539-549). To this end, the oligonucleotide can be conjugated to another molecule, (e.g., a peptide, hybridization triggered cross-linking agent, transport agent, or hybridization-triggered cleavage agent).

Also useful in the methods of the invention are molecular beacon oligonucleotide primer and probe molecules having at least one region that is complementary to a LVCa(v) nucleic acid of the invention, two complementary regions, one having a fluorophore and one a quencher such that the molecular beacon is useful for quantitating the presence of the LVCa(v) nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

siRNA Molecules

RNA interference (RNAi) is a process whereby double-stranded RNA (dsRNA) induces the sequence-specific degradation of homologous mRNA in animals and plant cells (Hutvagner and Zamore, 2002, Curr. Opin. Genet. Dev. 12:225-232; Sharp, 2001, Genes Dev. 15:485-490). In mammalian cells, RNAi can be triggered by approximately 21-nucleotide (nt) duplexes of small interfering RNA (siRNA) (Chiu et al., 2002, Mol. Cell. 10:549-561; Elbashir et al., 2001, Nature 411:494-498), or by micro-RNAs (mRNA), functional small-hairpin RNA (shRNA), or other dsRNAs which are expressed in vivo using DNA templates with RNA polymerase III promoters (Zeng et al., 2002, Mol. Cell 9:1327-1333; Paddison et al., 2002, Genes Dev., 16:948-958; Lee et al., 2002, Nature Biotechnol. 20:500-505; Paul et al., 2002, Nature Biotechnol. 20:505-508; Tuschl, 2002, Nature Biotechnol. 20:440-448; Yu et al., 2002, Proc. Natl. Acad. Sci. USA, 99:6047-6052; McManus et al., 2002, RNA 8:842-850; Sui et al., 2002, Proc. Natl. Acad. Sci. USA 99:5515-5520).

The nucleic acid molecules or constructs described herein include double-stranded RNA (dsRNA) molecules comprising 16-30, e.g., 16, 17, 18, 19, 20, 21, 22, 23, 24, 25, 26, 27, 28, 29, or 30 nucleotides in each strand, wherein one of the strands is substantially complementary to, e.g., at least 80% (or more, e.g., 85%, 90%, 95%, or 100%) complementary to, e.g., having 3, 2, 1, or 0 mismatched nucleotide(s), a target region, such as a target region that differs by at least one base pair between the wild type and mutant allele, e.g., a target region comprising the gain-of-function mutation, and the other strand is identical or substantially identical to the first strand. The dsRNA molecules of the invention can be chemically synthesized, or can be transcribed in vitro from a DNA template, or in vivo from an engineered RNA precursor, e.g., shRNA. The dsRNA molecules may be designed using methods known in the art, for example, by using the following protocol:

1. Beginning with the AUG start codon of, look for AA dinucleotide sequences; each AA and the 3′ adjacent 16 or more nucleotides are potential siRNA targets. The siRNA should be specific for a target region that differs by at least one base pair between the wild type and mutant allele, e.g., a target region comprising a gain of function mutation. The first strand should be complementary to this sequence, and the other strand is identical or substantially identical to the first strand. In one embodiment, the nucleic acid molecules are selected from a region of the target allele sequence beginning at least 50 to 100 nt downstream of the start codon of the sequence being targeted. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in some cases, the nucleic acid molecules have a 3′ overhang of 2 nucleotides, such as TT. The overhanging nucleotides can be either RNA or DNA. As noted above, it is desirable to choose a target region wherein the mutant:wild type mismatch is a purine:purine mismatch.

2. Using methods known in the art, compare the potential targets to the appropriate genome database (human, mouse, rat, etc.) and eliminate from consideration any target sequences with significant homology to other coding sequences. One such method for such sequence homology searches is known as BLAST, which is available at ncbi.nlm.nih.gov/BLAST.

3. Select one or more sequences that meet your criteria for evaluation.

Further general information about the design and use of siRNA can be found at, e.g., “The siRNA User Guide,” available at rockefeller.edu/labheads/tuschl/siRNA, dharmacon.com, ambion.com, or other resources known to those in the art.

Negative control siRNAs should have the same nucleotide composition as the selected siRNA, but without significant sequence complementarity to the targeted genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The siRNAs for use as described herein can be delivered to a cell by methods known in the art and as described above in using methods such as transfection using commercially available kits and reagents. Viral infection, e.g., using a lentivirus vector can be used.

The nucleic acid molecules described herein, including siRNA molecules, can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion Austin, Tex.). Additionally, an siRNA can be radiolabeled, e.g., using 3H, 32P, or other appropriate isotope.

Isolated LVCa(v) Polypeptides

An isolated LVCa(v) polypeptide, or a fragment thereof, e.g., a biologically active portion thereof, can be used as an immunogen or antigen to raise or test (or more generally to bind) anti-LVCa(v) antibodies useful in diagnostic assays and the preparation of therapeutic compositions. Such antibodies are also of commercial value as reagents for examining processes, expression, and localization related to the specific channel polypeptide to which the antibody binds. An LVCa(v) polypeptide can be isolated from cells or tissue sources using standard protein purification techniques. LVCa(v) polypeptides or fragments thereof can be produced by recombinant DNA techniques or synthesized chemically. The polypeptide can be expressed in systems, e.g., cultured cells, which result in substantially the same post-translational modifications present when the polypeptide is expressed in a native cell, or in systems which result in the alteration or omission of post-translational modifications, e.g., glycosylation or cleavage, present when expressed in a native cell.

Useful LVCa(v) polypeptides or fragments thereof differ from the corresponding LVCa(v) sequence, e.g., SEQ ID NO:21 or SEQ ID NO:22 (e.g., it differs by at least one, but by less than 15, 10, or 5 amino acid residues or by at least one residue but less than 20%, 15%, 10% or 5% of the residues in the sequence). Useful proteins include an amino acid sequence at least about 60%, 65%, 70%, 75%, 80%, 85%, 90%, 95%, 98%, or more homologous to an LVCa(v) sequence or fragment thereof (e.g., of an LVCa(v)1.1 or LVCa(v)1.3 sequence or novel exon as described herein).

Biologically active LVCa(v) polypeptides as fragments thereof can carry out at least one function associated with an LVCa(v). For example, participate in calcium metabolism. In some cases, polypeptide may be altered (compared to a naturally occurring polypeptide) and identified for its ability to effectively compete with the activity of the naturally occurring polypeptide, for example, the altered polypeptide may have an increased or deceased ability to modulate calcium influx relative to the naturally occurring polypeptide.

Chimeric or Fusion Proteins

In another aspect, the invention provides LVCa(v) chimeric or fusion proteins. As used herein, an LVCa(v) “chimeric protein” or “fusion protein” includes an LVCa(v) polypeptide linked to a non-LVCa(v) polypeptide. A “non-LVCa(v) polypeptide” refers to a polypeptide having an amino acid sequence corresponding to a protein that is not substantially homologous to the LVCa(v) protein, e.g., a protein that is different from the LVCa(v) protein and that is derived from the same or a different organism. The LVCa(v) polypeptide of the fusion protein can correspond to all or a portion e.g., a fragment described herein of an LVCa(v) amino acid sequence. In some embodiments, an LVCa(v) fusion protein includes at least one (e.g., two) biologically active portion of an LVCa(v) protein. The non-LVCa(v) polypeptide can be fused to the N-terminus or C-terminus of the LVCa(v) polypeptide.

The fusion protein can include a moiety that has a high affinity for a ligand. For example, the fusion protein can be a GST-LVCa(v) fusion protein in which the LVCa(v) sequence is fused to the C-terminus of a GST sequence. Such fusion proteins can be used to facilitate the purification of a recombinant LVCa(v). Alternatively, the fusion protein can be an LVCa(v) protein containing a heterologous signal sequence at its N-terminus. In certain host cells (e.g., mammalian host cells), expression, secretion, and/or placement into the cellular membrane of LVCa(v) can be increased through use of a heterologous signal sequence.

Fusion proteins can include all or a part of a serum protein, e.g., an IgG constant region, or human serum albumin.

The LVCa(v) fusion proteins of the invention can be incorporated into pharmaceutical compositions and administered to a subject in vivo. The LVCa(v) fusion proteins can be used to affect the bioavailability of an LVCa(v) substrate. LVCa(v) fusion proteins can be useful therapeutically for the treatment of disorders caused by, for example, (i) aberrant modification or mutation of a gene encoding an LVCa(v) protein; (ii) mis-regulation of the LVCa(v) gene; and (iii) aberrant post-translational modification of an LVCa(v) protein.

Moreover, the LVCa(v)-fusion proteins of the invention can be used as immunogens to produce anti-LVCa(v) antibodies in a subject, to purify LVCa(v) ligands and in screening assays to identify molecules that inhibit the interaction of LVCa(v) with an LVCa(v) substrate.

Expression vectors are commercially available that encode a fusion moiety (e.g., a GST polypeptide, V5, or FLAG). An LVCa(v)-encoding nucleic acid can be cloned into such an expression vector such that the fusion moiety is linked in-frame to the LVCa(v) polypeptide using methods known in the art.

Anti-LVCa(v) Antibodies

Anti-LVCa(v) antibodies can be used diagnostically and can be useful in therapeutic applications. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments, which can be generated by treating the antibody with an enzyme such as pepsin.

The antibody can be a polyclonal, monoclonal, recombinant, e.g., a chimeric or humanized, fully human, non-human (e.g., murine), or single chain antibody. In some embodiments, it has effector function and can fix complement. The antibody can be coupled to a toxin or imaging agent.

A full-length LVCa(v) protein or, antigenic peptide fragment of LVCa(v) can be used as an immunogen or can be used to identify anti-LVCa(v) antibodies made with other immunogens, e.g., cells, membrane preparations, and the like. In general, an antigenic peptide of LVCa(v) includes at least 8 amino acid residues of a sequence that is expressed only in a specific sequence LVCa(v) protein. For example, the amino acid residues used as an antigen can include a sequence encoded by exon A, exon B, exon A/B, a sequence that spans the splice junction of exons 11 and 13 of an LVCa(v)1.3, exon 33a, a sequence that spans the splice junction of exons 32 and 34 (exon 32-34) of an LVCa(v)1.3, exon A′, exon B′, exon A′/B′ or any other novel sequence disclosed herein. In general, the antigenic peptide includes at least 10 amino acid residues, for example, at least 15 amino acid residues, at least 20 amino acid residues, or at least 30 amino acid residues.

Epitopes encompassed by the antigenic peptide are generally regions of an LVCa(v) that are located on the surface of the protein, e.g., hydrophilic regions, as well as regions with high antigenicity. For example, an Emini surface probability analysis of the human LVCa(v) protein sequence can be used to indicate the regions that have a particularly high probability of being localized to the surface of the LVCa(v) protein and are, thus, likely to constitute surface residues useful for targeting antibody production.

Chimeric, humanized or completely human antibodies are desirable for applications that include repeated administration, e.g., therapeutic treatment (and some diagnostic applications) of human patients. Methods of humanizing antibodies are known in the art, e.g., Rader, et al., 1998, Proc. Nat. Acad. Sci USA 95:8910-8915; Abmaxis, Santa Clara, Calif.; Sierra Bio Source, Morgan Hill, Calif.

The anti-LVCa(v) antibody can be a single chain antibody. A single-chain antibody (scFV) may be engineered (see, for example, Colcher, et al., 1999, Ann. N.Y. Acad. Sci. 880:263-80; and Reiter, 1996, Clin. Cancer Res. 2:245-52). The single chain antibody can be dimerized or multimerized to generate multivalent antibodies having specificities for different epitopes of the same target LVCa(v) protein.

In some cases, the antibody has reduced or no ability to bind an Fc receptor, e.g., it is an isotype, subtype, fragment or other mutant, which does not support binding to an Fc receptor, e.g., it has a mutagenized or deleted Fc receptor binding region.

An anti-LVCa(v) antibody (e.g., monoclonal antibody) can be used to isolate an LVCa(v) polypeptide by methods known in the art, such as affinity chromatography or immunoprecipitation. Moreover, an anti-LVCa(v) antibody can be used to detect LVCa(v) protein (e.g., in a cellular lysate or cell supernatant) in order to evaluate the abundance and pattern of expression of the protein. Anti-LVCa(v) antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; an example of a luminescent material includes luminol; examples of bioluminescent materials include luciferase, luciferin, and aequorin, and examples of suitable radioactive materials include ¹²⁵I, ¹³¹I, ³⁵S or ³H.

Cells

LVCa(v) polypeptides are generally expressed in non-excitable cells such as cells associated with the immune system, e.g., lymphocytes including T cells, B cells, and mast cells. In the methods described herein, it is sometimes desirable to use a cell that naturally expresses or can express an LVCa(v) polypeptide such as a lymphocyte isolated from a subject, or a cultured lymphocyte cell such as a Jurkat cell (derived from human acute T cell leukemia; (Schneider et al., 1977, Int. J. Cancer 19: 621-626) or an HEK293 cell (derived from human embryonic kidney, Invitrogen, Carlsbad, Calif.). In some embodiments, a nucleic acid sequence encoding all or a portion of an LVCa(v) is introduced into non-human cells, including cells that are useful, e.g., for generating transgenic animals. Such non-human cells include DT40 chicken B cell line (an avian leukosis virus (ALV)-induced cell line; ATCC accession no. CRL-2111), mouse embryonic stem (ES) cells, and Xenopus oocytes. Other useful cell lines include B cell lines from non-humans and humans (e.g., Ramos, Daudi, and Raji cell lines) and other cell lines derived from immune system cells.

Recombinant Expression Vectors, Host Cells and Genetically Engineered Cells

Vectors, e.g., expression vectors, containing a nucleic acid encoding an LVCa(v) are useful for expressing an LVCa(v) polypeptide in vitro and in vivo. The recombinant expression vectors can be designed for expression of LVCa(v) polypeptides in prokaryotic or eukaryotic cells, for example, E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). For example, LVCa(v) proteins can be expressed in Xenopus oocytes or other cell types that are suitable for assaying the activity of LVCa(v) polypeptides. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in LVCa(v) activity assays, (e.g., direct assays or competitive assays described in detail below), or to generate antibodies specific for LVCa(v) proteins. To maximize recombinant protein expression in E. coli, the protein is expressed in a host bacterial strain with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

An LVCa(v) expression vector can be a yeast expression vector, a vector for expression in insect cells, e.g., a baculovirus expression vector, or a vector suitable for expression in mammalian cells. Useful vectors include recombinant viral gene transfer vectors such as adenovirus, adeno-associated virus, herpes virus, murine retrovirus and lentivirus vectors. Non-viral gene delivery systems can also be used for delivery of recombinant nucleic acids. Examples of non-viral gene delivery systems include naked DNA and DNA formulated with cationic lipids.

When used in mammalian cells, the expression vector's control functions can be provided by viral regulatory elements. For example, commonly used viral promoters are derived from polyoma, adenovirus 2, cytomegalovirus, and simian virus 40 (SV40).

A recombinant mammalian expression vector can be used to direct preferential expression of a nucleic acid in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally regulated promoters are also encompassed, for example, the murine hox promoters (Kessel and Gruss (1990) Science 249:374-379) and the α-fetoprotein promoter (Campes and Tilghman (1989) Genes Dev. 3:537-546).

Other useful recombinant expression vectors are designed to produce antisense nucleic acid molecules, including ribozymes. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes, see Weintraub et al., 1986, Reviews: Trends in Genetics 1(1).

Under some circumstances it is desirable to produce a host cell that includes a nucleic acid encoding all or part of an LVCa(v) nucleic acid molecule within a recombinant expression vector or an LVCa(v) nucleic acid molecule containing sequences which allow it to homologously recombine into a specific site of the host cell's genome. A host cell can be any prokaryotic or eukaryotic cell. For example, an LVCa(v) protein can be expressed in bacterial cells such as E. coli, insect cells, yeast, or mammalian cells (such as Chinese hamster ovary cells (CHO)) or COS cells. Other suitable host cells are known to those skilled in the art.

Vector DNA can be introduced into host cells via conventional transformation or transfection techniques, e.g., any art-recognized technique for introducing foreign nucleic acid (e.g., DNA) into a host cell, including calcium phosphate or calcium chloride co-precipitation, DEAE-dextran-mediated transfection, lipofection, or electroporation.

The host cell of the invention can be used to produce (i.e., express) an LVCa(v) protein, e.g., by culturing a host cell (into which a recombinant expression vector encoding an LVCa(v) protein has been introduced) in a suitable medium such that an LVCa(v) protein is produced and, optionally isolating a LVCa(v) protein from the medium or the host cell.

A cell or purified preparation of cells which include an LVCa(v) transgene, or that otherwise mis-express LVCa(v) can be used as a model for studying disorders (e.g., proliferative disorders) that are related to mutated or mis-expressed LVCa(v) alleles or for use in drug screening. The cell preparation can consist of human or non-human cells, e.g., rodent cells, such as mouse or rat cells; rabbit cells; or pig cells. In preferred embodiments, the cell or cells include an LVCa(v) transgene, e.g., a heterologous form of an LVCa(v), e.g., a gene derived from humans (in the case of a non-human cell). The LVCa(v) transgene can be mis-expressed, e.g., overexpressed or underexpressed. The cell or cells can include a gene that mis-expresses an endogenous LVCa(v), e.g., a gene the expression of which is disrupted, e.g., a knockout.

The expression characteristics of an endogenous gene within a cell, e.g., a cell line or microorganism, can be modified by inserting a heterologous DNA regulatory element into the genome of the cell such that the inserted regulatory element is operably linked to the endogenous LVCa(v) gene. For example, an endogenous LVCa(v) gene that is “transcriptionally silent,” e.g., not normally expressed, or expressed only at very low levels, may be activated by inserting a regulatory element that is capable of promoting the expression of a normally expressed gene product in that cell. Techniques such as targeted homologous recombination, can be used to insert the heterologous DNA as described in, e.g., Chappel, U.S. Pat. No. 5,272,071; WO 91/06667, published on May 16, 1991; U.S. Pat. No. 6,270,989.

In general, CMV promoter-containing vectors are used although retroviral based vectors are also useful. Such vectors are commercially available and some are described herein.

Screening Assays

The invention provides screening methods (also referred to herein as “assays”) for identifying modulators, i.e., candidate compounds or agents (e.g., proteins, peptides, peptidomimetics, peptoids, oligonucleotides (such as siRNA or anti-sense RNA), small non-nucleic acid organic molecules, small inorganic molecules, or other drugs) that bind to LVCa(v) proteins, have an inhibitory (or stimulatory) effect on, for example, LVCa(v) expression or LVCa(v) activity, or have a stimulatory or inhibitory effect on, for example, the expression or activity of a LVCa(v) substrate. Such substrates can include Ca2+ and other (e.g., non-∝) subunits of calcium channels (Catterall, 2000, supra). In general, the novel calcium channel subunits described herein are incorporated into channel complexes, for example, in place of a corresponding neuronal channel subunit (such as an ∝ subunit). Compounds thus identified can be used to modulate the activity of target gene products (e.g., LVCa(v) a polypeptides) either directly or indirectly in a therapeutic protocol, to elaborate the biological function of the target gene product, or to identify compounds that disrupt normal target gene interactions.

Compounds that inhibit the activity or expression of an LVCa(v) are useful in the treatment of proliferative disorders involving cells that express an LVCa(v). Such disorders include cancers, hyperproliferative disorders, and neoplastic disorders. Such disorders also include disorders involving lymphocytes, e.g., cancers such as leukemias, autoimmune diseases (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, type II diabetes, uticaria, restenosis, and allergy such as, atopic allergy.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. In general, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, 1991, Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Sternberg disease.

Cells or tissues affected by these disorders can be used in screening methods, e.g., to test whether an agent that modulates expression of activity of an LVCa(v) can reduce proliferation of affected cells.

In one embodiment, the invention provides assays for screening candidate or test compounds that bind to or modulate the activity of an LVCa(v) protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone, which are resistant to enzymatic degradation but that nevertheless remain bioactive; see, e.g., Zuckermann, et al., 1994 J. Med. Chem. 37: 2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., 1993, Proc. Natl. Acad. Sci. USA. 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam (1991) Nature 354:82-84), chips (Fodor, 1993, Nature 364:555-556), bacteria (Ladner, U.S. Pat. No. 5,223,409), spores (Ladner, U.S. Pat. No. 5,223,409), plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. 87:6378-6382; Felici, 1991, J. Mol. Biol. 222:301-310; and Ladner supra.).

The compounds that can be screened by the methods described herein include, but are not limited to, any small molecule compound libraries derived from natural and/or synthetic sources, small non-nucleic acid organic molecules, small inorganic molecules, peptides, peptoids, peptidomimetics, oligonucleotides (e.g., siRNA, antisense RNA, aptamers such as those identified using SELEX), and oligonucleotides containing synthetic components.

In one embodiment, an assay is a cell-based assay in which a cell that expresses an LVCa(v) protein or biologically active portion thereof is contacted with a test compound, and the ability of the test compound to modulate LVCa(v) activity is determined. Determining the ability of the test compound to modulate LVCa(v) activity can be accomplished by monitoring, for example, changes in calcium flux in the cell or by testing downstream effects of modulating calcium flux such as IL-2 expression or NFAT (nuclear factor of activated T cells, a transcription factor regulating IL-2). Methods of testing such downstream effects are known in the art and include modulation of cell proliferation and cell growth. For example, a compound that decreases the number of LVCa(v) molecules in a cell or affects the function of an LVCa(v) channel may decrease cellular proliferation.

In some cases, the cell used in such assays is one that does not normally express the channel protein of interest, e.g., a Xenopus oocyte or immune system cell or derivative thereof that expresses a recombinant LVCa(v) protein, polypeptide or biologically active portion thereof. In general, recombinant expression that results in increased expression of the LVCa(v) compared to a corresponding cell that does not express recombinant LVCa(v), is referred to as “overexpression” of the LVCa(v). Alternatively, the cell can be of mammalian origin. The cell can also be a cell that expresses the channel but in which such channel activity can be distinguished from other calcium channel activity, for example, by comparison with controls. The ability of the test compound to modulate LVCa(v) binding to a compound, e.g., an LVCa(v) substrate, or to bind to LVCa(v) can also be evaluated. This can be accomplished, for example, by coupling the compound, e.g., the substrate, with a radioisotope or enzymatic label such that binding of the compound, e.g., the substrate, to LVCa(v) can be determined by detecting the labeled compound, e.g., substrate, in a complex. Alternatively, LVCa(v) could be coupled with a radioisotope or enzymatic label to monitor the ability of a test compound to modulate LVCa(v) binding to an LVCa(v) substrate in a complex. For example, compounds (e.g., LVCa(v) substrates) can be labeled with 125I, 35S, 14C, or 3H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product. In general, it is not necessary to co-express auxiliary subunits associated with L-type channels although in some embodiments such co-expression is performed (e.g., co-expression of a β subunit.

An example of a screening assay for a compound that specifically modulates activity of an LVCa(v) polypeptide is as follows. Incubate a cell that expresses the LVCa(v) polypeptide of interest (e.g., a Jurkat cell or an HEK293 cell) with a test compound for a time sufficient for the compound to have an effect on transcription or activity (e.g., for at least 1 minute, 10 minutes, 1 hour, 3 hours, 5 hours, or 24 or more hours. Such times can be determined experimentally. The concentration of the test compound can also be varied (e.g., from 1 nM-100 μM, 10 nM to 10 μM or, 1 nM to 10 μM). Inhibition of calcium influx in the presence and absence of the test compound is then assayed using methods known in the art. For example, fura-2, Indo-1, Fluo-3, or Rho-2 can be used to assay calcium flux. Other methods can be used as assays of inhibition. For example, a test compound is considered to have a significant impact on influx if any one or more of the following criteria are met:
a. there is a direct inhibition of Icrac as measured by patch clamp;
b. there is inhibition of downstream signaling functions such as calcineurin activity, NFAT, and/or IL-2 production; or
c. there are modifications in activation-induced cell proliferation, differentiation and/or apoptotic signaling pathways.

Direct testing of the effect of a test compound on that an activity of a specific LVCa(v) polypeptide can be accomplished using, e.g., single channel patch clamping to measure Icrac. This method can be used in screening assays as a second step after testing for general effects on calcium influx or as a second step after identifying a test compound as affecting expression of an LVCa(v) mRNA or polypeptide. Alternatively, direct testing can be used as a first step in a multiple step assay or in single step assays.

In another method of determining whether a test compound is binding L-type channels, competition with DM-BODIPY dyes is used. Such assays employ DM-BODIPY (Molecular Probes, Eugene, R.), a dihydropyridine analog that is covalently attached to the BODIPY dye. The DM-BODIPY dye binds to L-type calcium channels on the cell surface and can be assessed by flow-cytometry. Accordingly, a test compound can be used in a competition assay with a DM-BODIPY dye. If the test compound is able to compete for binding of the DM-BODIPY dye to a cell expressing an LVCa(v) polypeptide, the test compound is a candidate compound for binding to the LVCa(v) polypeptide and being a modulator of the activity of that channel.

The ability of a compound (e.g., an LVCa(v) substrate) to interact with LVCa(v) with or without the labeling of any of the interactants can be evaluated. For example, a microphysiometer can be used to detect the interaction of a compound with LVCa(v) without the labeling of either the compound or the LVCa(v) (McConnell et al., 1992, Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and LVCa(v) polypeptide.

In yet another embodiment, a cell-free assay is provided in which a LVCa(v) protein or biologically active portion thereof is contacted with a test compound and the ability of the test compound to bind to the LVCa(v) protein or biologically active portion thereof is evaluated. Preferred biologically active portions of the LVCa(v) proteins to be used in assays of the present invention include fragments that participate in interactions with non-LVCa(v) molecules, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label is selected such that a first donor molecule's emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the LVCa(v) protein to bind to a target molecule can be accomplished using real-time Biomolecular Interaction Analysis (BIA) (see, e.g., Sjolander and Urbaniczky, 1991, Anal. Chem. 63:2338-2345 and Szabo et al., 1995, Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product (e.g., LVCa(v) polypeptide or the test substance is anchored onto a solid phase. The target gene product/test compound complexes anchored on the solid phase can be detected at the end of the reaction. In general, the target gene product can be anchored onto a solid surface, and the test compound, (which is not anchored), can be labeled, either directly or indirectly, with detectable labels discussed herein.

It may be desirable to immobilize an LVCa(v), an anti-LVCa(v) antibody or its target molecule to facilitate separation of complexed from non-complexed forms of one or both of the proteins, as well as to accommodate automation of the assay. Binding of a test compound to an LVCa(v) protein, or interaction of an LVCa(v) protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/LVCa(v) fusion proteins or glutathione-S-transferase/target fusion proteins can be adsorbed onto glutathione Sepharose™ beads (Sigma Chemical, St. Louis, Mo.) or glutathione-derivatized microtiter plates, which are then combined with the test compound or the test compound and either the non-adsorbed target protein or LVCa(v) protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of LVCa(v) binding or activity determined using standard techniques.

Other techniques for immobilizing either LVCa(v) protein or a target molecule on matrices include using conjugation of biotin and streptavidin. Biotinylated LVCa(v) protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemicals).

To conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

This assay is performed utilizing antibodies reactive with LVCa(v) protein or target molecules but which do not interfere with binding of the LVCa(v) protein to its target molecule. Such antibodies can be derivatized to the wells of the plate, and unbound target or LVCa(v) protein trapped in the wells by antibody conjugation. Methods for detecting such complexes, in addition to those described above for the GST-immobilized complexes, include immunodetection of complexes using antibodies reactive with the LVCa(v) protein or target molecule, as well as enzyme-linked assays which rely on detecting an enzymatic activity associated with the LVCa(v) protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, 1993, Trends Biochem. Sci. 18:284-7); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (see, e.g., Heegaard, 1998, J. Mol. Recognit. 11:141-8; Hage and Tweed, 1997, J. Chromatogr. B. Biomed. Sci. Appl. 699:499-525). Further, fluorescence energy transfer may also be conveniently utilized, as described herein, to detect binding without further purification of the complex from solution.

The assay can include contacting the LVCa(v) protein or biologically active portion thereof with a known compound that binds LVCa(v) to form an assay mixture, contacting the assay mixture with a test compound, and determining the ability of the test compound to interact with an LVCa(v) polypeptide, wherein determining the ability of the test compound to interact with an LVCa(v) protein includes determining the ability of the test compound to preferentially bind to LVCa(v) or biologically active portion thereof, or to modulate the activity of a target molecule, as compared to the known compound.

To the extent that LVCa(v) can, in vivo, interact with one or more cellular or extracellular macromolecules, such as proteins, inhibitors of such an interaction are useful. Such interacting molecules include Ca2+ and subunits of the calcium channel complex as well as signaling molecules that directly interact with the channel such as protein kinase A (PKA) and protein kinase C (PKC) homogeneous assay can be used can be used to identify inhibitors. For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, an LVCa(v) polypeptide can be used as a “bait protein” in a two-hybrid assay or three-hybrid assay (see, e.g., U.S. Pat. No. 5,283,317; Zervos et al., 1993, Cell 72:223-232; Madura et al., 1993, J. Biol. Chem. 268:12046-12054; Bartel et al., 1993, Biotechniques 14:920-924; Iwabuchi et al., 1993, Oncogene 8:1693-1696; and Brent WO94/10300), to identify other proteins, that bind to or interact with LVCa(v) (“LVCa(v)-binding proteins” or “LVCa(v)-bp”) and are involved in LVCa(v) activity. Such LVCa(v)-bps can be activators or inhibitors of signals by the LVCa(v) proteins or LVCa(v) targets as, for example, downstream elements of an LVCa(v)-mediated signaling pathway, e.g., IL-2 expression or activity.

Modulators of LVCa(v) expression can also be identified. For example, a cell or cell free mixture is contacted with a candidate compound and the expression of an LVCa(v) mRNA or protein evaluated relative to the level of expression of an LVCa(v) mRNA or protein in the absence of the candidate compound. When expression of an LVCa(v) mRNA or protein is greater in the presence of the candidate compound than in its absence, the candidate compound is identified as a stimulator of LVCa(v) mRNA or protein expression. Alternatively, when expression of LVCa(v) mRNA or protein is less (i.e., statistically significantly less) in the presence of the candidate compound than in its absence, the candidate compound is identified as an inhibitor of LVCa(v) mRNA or protein expression. The level of LVCa(v) mRNA or protein expression can be determined by methods described herein for detecting an LVCa(v) mRNA or protein.

A modulating agent can be identified using a cell-based or a cell-free assay, and the ability of the agent to modulate the activity of a LVCa(v) protein can be confirmed in vivo, e.g., in an animal such as an animal model for a disease (e.g., an animal with leukemia or autoimmune disease or an animal harboring a xenograft from an animal (e.g., human) or cells from a cancer resulting from a leukemia or other lymphocytic disorder, or cells from a leukemia or other lymphocytic disorder cell line.

This invention further pertains to novel agents identified by the above-described screening assays. Accordingly, it is within the scope of this invention to further use an agent identified as described herein (e.g., a LVCa(v)-modulating agent, an antisense LVCa(v) nucleic acid molecule, a LVCa(v)-specific antibody, or a LVCa(v)-binding partner) in an appropriate animal model (such as those described above) to determine the efficacy, toxicity, side effects, or mechanism of action, of treatment with such an agent. Furthermore, novel agents identified by the above-described screening assays can be used for treatments as described herein.

Animal models that are useful include animal models of leukemia and autoimmune disorders. Examples of such animal models are known in the art and can be obtained from commercial sources, e.g., the Jackson Laboratory (Bar Harbor, Me.) or generated as described in the relevant literature. Examples of animals useful for such studies include mice, rats, dogs, cats, sheep, rabbits, and goats.

Compounds that Modulate LVCa(v) Expression or Activity

Compounds that affect LVCa(v) expression or activity can be identified as described above or using other methods known in the art. The modulator compounds can be novel, compounds not previously identified as having any type of activity as a calcium channel modulator, or a compound previously known to modulate calcium channels, including L-type channels, but that is used at a concentration not previously known to be effective for modulating calcium influx. Such compounds include dihydropyridines, phenylalkylamine, benzodiazepine, benzothiazapine, diarylaminopropylamine ether, and benzimidazole-substituted tetralin. The concentrations at which such compounds are used, e.g., to modulate expression or activity of an LVCa(v), can be, e.g., 0.1-100 μM (e.g., 1-10 μM, 10-100 μM, 0.1-1 μM, or 0.1-10 μM) in cultured cells or tissue explants.

LVCa(v) proteins may also serve as scaffolding areas and as nexus proteins for signaling molecules. Such activities, when associated with and LVCa(v) protein, can be exploited in functional assays of LVCa(v) proteins or polypeptides.

Transgenic Animals

The invention provides non-human transgenic animals that are engineered to overexpress an LVCa(v), ectopically express an LVCa(v), express a mutant LVCa(v), or be knocked out for expression of an LVCa(v). Such animals and cell lines derived from such animals are useful for studying the function and/or activity of an LVCa(v) protein and for identifying and/or evaluating modulators of LVCa(v) activity. An animal that overexpresses an LVCa(v) polypeptide is useful, e.g., for testing the effects of candidate compounds for modulating the activity of the LVCa(v) polypeptide and assessing the effect of the compound in vivo.

As used herein, a “transgenic animal” is a non-human animal, in general, a mammal, for example, a rodent such as a rat or mouse, in which one or more of the cells of the animal include a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which is in most cases integrated into or occurs in the genome of the cells of a transgenic animal. A transgene can direct the expression of an encoded gene product in one or more cell types or tissues of the transgenic animal; other transgenes, e.g., a knockout, reduce expression. Thus, a transgenic animal can be one in which an endogenous LVCa(v) gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of an LVCa(v) protein to particular cells. A transgenic founder animal can be identified based upon the presence of an LVCa(v) transgene in its genome and/or expression of LVCa(v) mRNA in tissues or cells of the animals. A transgenic founder animal can then be used to breed additional animals carrying the transgene. Moreover, transgenic animals carrying a transgene encoding an LVCa(v) protein can further be bred to other transgenic animals carrying other transgenes.

LVCa(v) proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

In one non-limiting example, a mouse is engineered to express an LVCa(v) polypeptide (e.g., an LVCa(v)1.3 or LVCa(v)1.1) using a T cell-specific promoter such as an LCK promoter using methods known in the art (e.g., Zhang et al., 2002, Nat. Immunol. 3:749-755). Engineered animals can be identified using known methods of identifying the presence of a transgene in cells and by assaying a cell sample (e.g., T cells) for the overexpression of the LVCa(v) (for example, using immunocytochemistry) or by assaying calcium flux in a cell from the sample. Such transgenic animals are useful, e.g., for testing compounds for their ability to inhibit LVCa(v)1.3-mediated cell proliferation.

The invention also includes a population of cells from a transgenic animal. Methods of developing primary, secondary, and immortal cell lines from such animals are known in the art.

Predictive Medicine

The present invention also pertains to the field of predictive medicine in which diagnostic assays, prognostic assays, and monitoring clinical trials are used for prognostic (predictive) purposes to thereby treat an individual.

Generally, the invention provides a method of determining if a subject is at risk for a disorder related to a lesion in or the misexpression of a gene that encodes LVCa(v). In general, such lesions are in exons that are specifically expressed in LVCa(v) and not in other L-type channel polypeptides.

Such disorders include, e.g., a disorder associated with the misexpression of gene encoding an LVCa(v).

The method includes one or more of the following detection steps:
(i) detecting, in a tissue of the subject, the presence or absence of a mutation that affects the expression (e.g., increases or decreases expression compared to a wild type or normal control subject) of the LVCa(v) mRNA sequence, or detecting the presence or absence of a mutation in a region that controls the expression of the gene in a lymphocyte, e.g., a mutation in the 5′ control region, or detecting a mutation in an L-type channel exon (or exons) that is specifically expressed in an LVCa(v) (e.g., exon A, exon B, exon A/B, exon 33a, exon A′, and exon B′, or exon A′/B′;
(ii) detecting, in a tissue of the subject, the presence or absence of a mutation that alters the structure of the L-type channel gene such that LVCa(v) will be misexpressed;
(iii) detecting, in a tissue of the subject, the misexpression of the LVCa(v) sequence, at the mRNA level, e.g., detecting a non-wild type level of an mRNA or an inappropriately spliced LVCa(v); or
(iv) detecting, in a tissue of the subject, the misexpression of the gene, at the protein level, e.g., detecting a non-wild type level of an LVCa(v) polypeptide.

In some embodiments the method includes: ascertaining the existence of at least one of: a deletion of one or more nucleotides from an exon expressed preferentially in a LVCa(v) sequence (e.g., exon A, exon B, exon 33a, exon A′, or exon B′); an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from an LVCa(v), or naturally occurring mutants thereof or 5′ or 3′ flanking sequences naturally associated with the LVCa(v) gene; (ii) exposing the probe/primer to nucleic acid of the tissue; and detecting, by hybridization, e.g., in situ hybridization, of the probe/primer to the nucleic acid, the presence or absence of the genetic lesion.

In some cases, detecting the misexpression includes ascertaining the existence of at least one of: an alteration in the level of a messenger RNA transcript of the LVCa(v) gene; the presence of a non-wild type splicing pattern of a messenger RNA transcript of the gene; or a non-wild type level of LVCa(v).

Methods of the invention can be used prenatally or to determine if a subject's offspring will be at risk for a disorder.

The method includes determining the structure of a gene encoding an LVCa(v), an abnormal structure being indicative of risk for the disorder. The entire structure of the gene need not be determined. In general, the determination will be by examining those exons that are specifically expressed in LVCa(v) and not in other known L-type channels.

The method includes contacting a sample from the subject with an antibody to an LVCa(v) protein or a nucleic acid that specifically hybridizes with portions of the L-type channel gene that are specifically expressed in an LVCa(v).

Diagnostic and Prognostic Assays

The presence, level, or absence of a LVCa(v) protein or nucleic acid in a biological sample can be evaluated by obtaining a biological sample from a test subject and contacting the biological sample with a compound or an agent capable of detecting LVCa(v) protein or nucleic acid (e.g., mRNA, genomic DNA) that encodes LVCa(v) protein such that the presence of LVCa(v) protein or nucleic acid is detected in the biological sample. The term “biological sample” includes tissues, cells and biological fluids isolated from a subject, as well as tissues, cells and fluids present within a subject. In general the biological sample is blood. The level of expression of the LVCa(v) gene can be measured in a number of ways, including, but not limited to: measuring an LVCa(v) mRNA; measuring the amount of an LVCa(v) protein; or measuring the activity of an LVCa(v) protein. The measurement can be of the expression of a specific LVCa(v) or expression of several species of LVCa(v) (e.g., by assaying expression of an exon that is in common between more than one LVCa(v)).

The level of an LVCa(v) mRNA in a cell can be determined by in situ or by in vitro formats.

Isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length LVCa(v) nucleic acid, such as the nucleic acid of LV1Ca(v)1.3 or LV2Ca(v)1.3 that contains the complete coding sequence, or a portion thereof, such as an oligonucleotide of at least 7, 15, 30, 50, 100, 250 or 500 nucleotides in length and sufficient to specifically hybridize under stringent conditions to LVCa(v) mRNA or genomic DNA. The sequence detected can be in the 3′ untranslated or 5′ untranslated regions of an LVCa(v) nucleic acid molecule. In general, the sequence detected is a portion of an LVCa(v) sequence that is specifically or preferentially expressed in a particular cell type. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of an LVCa(v) mRNA.

The level of a LVCa(v) mRNA in a sample can be evaluated with nucleic acid amplification, e.g., by RT-PCR (Mullis, 1987, U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the LVCa(v) gene being analyzed.

In another embodiment, the methods further contacting a control sample with a compound or agent that can be used to detect LVCa(v) mRNA, or segments of genomic DNA that are specific to a LVCa(v), and comparing the presence of LVCa(v) mRNA or genomic DNA in the control sample with the presence of LVCa(v) mRNA or genomic DNA in the test sample. A variety of methods can be used to determine the level of an LVCa(v) protein. In general, these methods include contacting an agent that selectively binds to the protein, such as an antibody with a sample, to evaluate the level of protein in the sample. In a preferred embodiment, the antibody bears a detectable label. Antibodies can be polyclonal, or monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect LVCa(v) protein in a biological sample in vitro as well as in vivo. In vitro techniques for detection of LVCa(v) protein include enzyme linked immunosorbent assays (ELISAs), immunoprecipitations, immunofluorescence, enzyme immunoassay (EIA), radioimmunoassay (RIA), and Western blot analysis. In vivo techniques for detection of LVCa(v) protein include introducing into a subject a labeled anti-LVCa(v) antibody. For example, the antibody can be labeled with a radioactive marker whose presence and location in a subject can be detected by standard imaging techniques.

In another embodiment, the methods further include contacting the control sample with a compound or agent capable of detecting LVCa(v) protein, and comparing the presence of LVCa(v) protein in the control sample with the presence of LVCa(v) protein in the test sample.

The invention also includes kits for detecting the presence of LVCa(v) in a biological sample. For example, the kit can include a compound or agent capable of detecting LVCa(v) protein or mRNA in a biological sample; and a standard. The compound or agent can be packaged in a suitable container. The kit can further comprise instructions for using the kit to detect LVCa(v) protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein-stabilizing agent. The kit can also includes components necessary for detecting the detectable agent (e.g., an enzyme or a substrate). The kit can also contain a control sample or a series of control samples that can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted LVCa(v) expression or activity. As used herein, the term “unwanted” includes an unwanted phenomenon involved in a biological response such as pain or deregulated cell proliferation.

In one embodiment, a disease or disorder associated with aberrant or unwanted LVCa(v) expression or activity is identified. A test sample is obtained from a subject and LVCa(v) protein or nucleic acid (e.g., mRNA or genomic DNA) is evaluated, wherein the level, e.g., the presence or absence, of LVCa(v) protein or nucleic acid is diagnostic for a subject having or at risk of developing a disease or disorder associated with aberrant or unwanted LVCa(v) expression or activity. As used herein, a “test sample” refers to a biological sample obtained from a subject of interest, including a biological fluid (e.g., blood or buffy coat), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted LVCa(v) expression or activity. For example, such methods can be used to determine whether a subject can be effectively treated with an agent for an LVCa(v)-associated disorder.

The methods of the invention can also be used to detect genetic alterations in a LVCa(v) gene, thereby determining if a subject with the altered gene is at risk for a disorder characterized by misregulation in LVCa(v) protein activity or nucleic acid expression. In general, the methods include detecting, in a sample from the subject, the presence or absence of a genetic alteration characterized by at least one of an alteration affecting the integrity of a gene encoding a LVCa(v)-protein, or the mis-expression of the LVCa(v) gene. For example, such genetic alterations can be detected by ascertaining the existence of at least one of the following types of alterations/modifications as:
(1) a deletion of one or more nucleotides from a LVCa(v) gene;
(2) an addition of one or more nucleotides to a LVCa(v) gene;
(3) a substitution of one or more nucleotides of a LVCa(v) gene,
(4) a chromosomal rearrangement of a LVCa(v) gene;
(5) an alteration in the level of a messenger RNA transcript of a LVCa(v) gene,
(6) aberrant modification of a LVCa(v) gene, such as of the methylation pattern of the genomic DNA,
(7) the presence of a non-wild type splicing pattern of a messenger RNA transcript of a LVCa(v) gene,
(8) a non-wild type level of a LVCa(v)-protein,
(9) allelic loss of a LVCa(v) gene, and 10) inappropriate post-translational modification of a LVCa(v)-protein.

In the present context, an LVCa(v) gene refers to the genomic sequence that is required to produce a normal LVCa(v) mRNA.

An alteration can be detected without a probe/primer in a polymerase chain reaction, such as anchor PCR or RACE PCR, or, alternatively, in a ligation chain reaction (LCR), the latter of which can be particularly useful for detecting point mutations in a portion of the gene specific to an LVCa(v). This method can include the steps of collecting a sample of cells from a subject, isolating nucleic acid (e.g., genomic, mRNA or both) from the sample, contacting the nucleic acid sample with one or more primers which specifically hybridize to a LVCa(v)-specific portion of a gene under conditions such that hybridization and amplification of the LVCa(v)-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a gene encoding an LVCa(v) from a sample cell can be identified by detecting alterations in restriction enzyme cleavage patterns. In general, the alterations are detected in regions of the gene encoding cell-type specific or preferentially expressed sequences. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in an LVCa(v) nucleic acid sequence can be identified by hybridizing a sample and control nucleic acids, e.g., DNA or RNA, two-dimensional arrays, e.g., chip-based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin et al., 1996, Human Mutation 7: 244-255; Kozal et al,.1996, Nature Medicine 2: 753-759). For example, genetic mutations in LVCa(v) can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a gene encoding an LVCa(v) and detect mutations by comparing the sequence of the sample LVCa(v) sequence with the corresponding wild-type (control) sequence. In general, exons are sequenced that are specifically expressed in the LVCa(v) and not in other species encoded by the gene. Automated sequencing procedures can be utilized when performing the diagnostic assays, 1995, Biotechniques 19:448), including sequencing by mass spectrometry.

Other methods for detecting mutations in sequences encoding an LVCa(v) include methods in which protection from cleavage agents is used to detect mismatched bases in RNA/RNA or RNA/DNA heteroduplexes (Myers et al., 1985, Science 230:1242; Cotton et al., 1988, Proc. Natl. Acad. Sci. USA 85:4397; Saleeba et al., 1992, Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in LVCa(v) cDNAs obtained from samples of cells. For example, the mutY enzyme of E. coli cleaves A at G/A mismatches and the thymidine DNA glycosylase from HeLa cells cleaves T at G/T mismatches (Hsu et al., 1994, Carcinogenesis 15:1657-1662; U.S. Pat. No. 5,459,039).

In other embodiments, alterations in electrophoretic mobility will be used to identify mutations in LVCa(v) genes. For example, single strand conformation polymorphism (SSCP) may be used to detect differences in electrophoretic mobility between mutant and wild type nucleic acids (Orita et al., 1989, Proc. Natl. Acad. Sci. USA: 86:2766, see also Cotton, 1993, Mutat. Res. 285:125-144; and Hayashi, 1992, Genet. Anal. Tech. Appl. 9:73-79). Single-stranded DNA fragments of sample and control LVCa(v) nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., 1991, Trends Genet 7:5).

In yet another embodiment, the movement of mutant or wild-type fragments in polyacrylamide gels containing a gradient of denaturant is assayed using denaturing gradient gel electrophoresis (DGGE) (Myers et al., 1985, Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, 1987, Biophys. Chem. 265:12753).

Examples of other techniques for detecting point mutations include, but are not limited to, selective oligonucleotide hybridization, selective amplification, or selective primer extension (Saiki et al., 1986, Nature 324:163-166; Saiki et al., 1989, Proc. Natl. Acad. Sci. USA 86:6230-6234).

Alternatively, allele specific amplification technology, which depends on selective PCR amplification, may be used in conjunction with the instant invention. Oligonucleotides used as primers for specific amplification may carry the mutation of interest in the center of the molecule (so that amplification depends on differential hybridization) (Gibbs et al., 1989, Nucleic Acids Res. 17:2437-2448) or at the extreme 3′ end of one primer where, under appropriate conditions, mismatch can prevent, or reduce polymerase extension (Prossner, 1993, Tibtech 11:238). In addition it may be desirable to introduce a novel restriction site in the region of the mutation to create cleavage-based detection (Gasparini et al., 1992, Mol. Cell Probes 6:1). It is anticipated that in certain embodiments amplification may also be performed using Taq ligase for amplification (Barany, 1991, Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a LVCa(v) gene.

Pharmaceutical Compositions

The nucleic acid and polypeptides, fragments thereof, as well as anti-LVCa(v) antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, in general, about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, between 2 to 8 weeks, between about 3 to 7 weeks, or for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, can include a series of treatments.

For antibodies, the dosage is generally 0.1 mg/kg of body weight (e.g., 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al., 1997, J. Acquired Immune Deficiency Syndromes and Human Retrovirology 14:193.

The present invention encompasses agents that modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, sex, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents (e.g., vincristine and vinblastine).

The conjugates described herein can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, γ-interferon, nerve growth factor, platelet derived growth factor, tissue plasminogen activator; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Compounds described herein that modulate expression or activity of as described above may be used for the preparation of a medicament for use in any of the methods of treatment described herein.

Uses

A number of embodiments of the invention have been described. Nevertheless, it will be understood that various modifications may be made without departing from the spirit and scope of the invention.

### EXAMPLES

The invention is further illustrated by the following examples. The examples are provided for illustrative purposes only. They are not to be construed as limiting the scope or content of the invention in any way.

### Example 1

### Identification of Alternatively Spliced Ca(v)1.3 Sequences Expressed in Lymphocyte Cell Line

Identification of Ca(v)1.3 Sequence with a Novel 5′ Sequence

Novel splice variants of the L-type calcium channel (termed LVCa(v)) were identified in Jurkat cells using RT-PCR. RT-PCR of partial and full-length sequences was performed using the Superscript™ II (Invitrogen, Carlsbad, Calif.) and Advantage™ II enzymes (BD Biosciences, San Jose, Calif.) according to the manufacturer's instructions. PCR reactions were performed in a Biometra TGradient machine (Biometra, Horsham, Calif.). Sequence identification was performed using an ABI 310 sequencer.

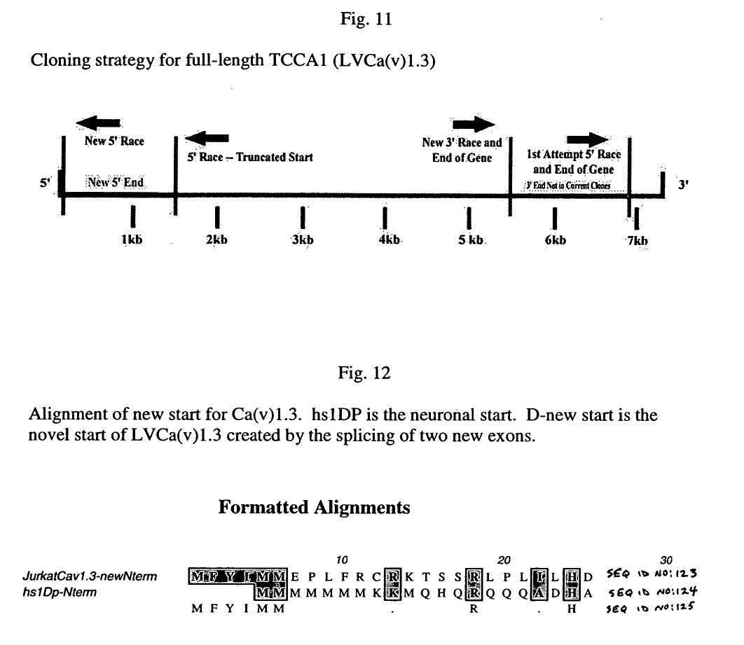

Briefly, 5′ and 3′ RACE was used to identify Ca(v)1.3 sequence expressed in Jurkat cells. 5′RACE was performed using a Generacer kit (Invitrogen) following the manufacturer's instructions. The kit primers were used and gene-specific oligonucleotide primers were designed to initiate in exon 2 of Ca(v)1.3. The GenBank Accession number for Ca(v)1.3 is NM_{—}000720 (Homo sapiens calcium channel, voltage-dependent, L type, alpha 1D subunit (CACNA1D), mRNA). The intron/exon structure of neuronal Ca(v)1.3 is known (see accession number NT_{—}005986, Homo sapiens chromosome 3 genomic contig). Oligonucleotides used in the following protocols were purchased from Invitrogen and were designed based either upon sequences available in public databases or upon sequences identified as described herein. The gene-specific primer used to initiate the RACE reaction in exon 2 of Ca(v)1.3 was 5′CCCTGTTTTTTGCTCTTGGCGTATTGC3′ (D-ex2Race1; SEQ ID NO:29) and the nested primer was 5′CTTGGCGTATTGCTGACGTTTTCTTTG3′ (D-ex2Race1nest; SEQ ID NO:30). The RACE assays were performed on polyA mRNA that was isolated from Jurkat cells (FastTrack 2.0 kit; Invitrogen). Both primers yielded the same product, a novel lymphocyte-specific 5′ end of a Ca(v)1.3 sequence. A schematic drawing of the cloning strategy is shown in FIG. 11.

Two different predicted translations of the start sites and initial amino acid sequence were identified in the novel sequence; NH₃-MFYIMMEPLFRCRKTSSRLPLILHD . . . —COOH (SEQ ID NO:31) and NH₃-MMEPLFRCRKTSSRLPLILHD . . . —COOH (SEQ ID NO:32). Some allelic variation can occur in these sequences. For example, the start sequence can be NH3-MFYIMMEPLFRCRKTSSRLPLILHD . . . —COOH (SEQ. ID NO:33).

To summarize, the novel Ca(v)1.3 RNA sequences have a novel exon region that is transcribed from a region that is located about 170 kb upstream of exon 2 on the genomic contig, NT_{—}005986. The novel exon sequences are in a region of the genome that is otherwise noncoding. In the previously identified forms of Ca(v)1.3 sequences, exon 1 is transcribed from genomic sequence that is about 2 kb upstream of exon 2.

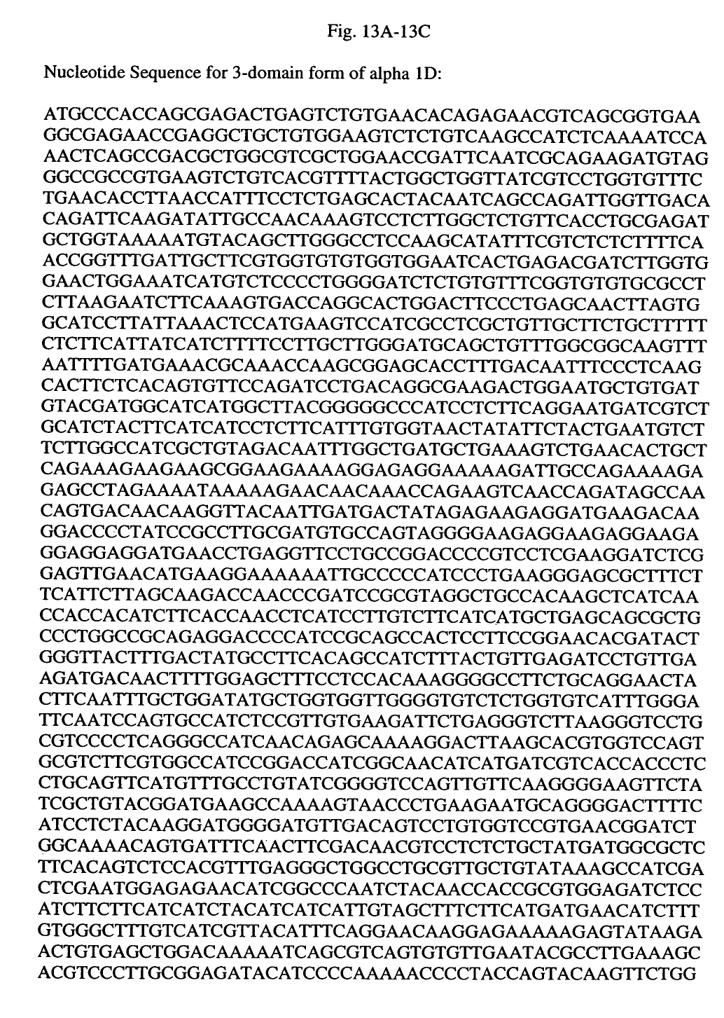

Two novel exons (herein termed exon A and exon B) are combined to create a start for novel LVCa(v)1.3 transcripts. The novel exon(s) (together termed exon A/B) replace the previously identified exon 1 of L-type channels in the novel Ca(v)1.3 transcript. As discussed above, the novel exon A/B is created by an alternate splicing event, about 170 kb upstream of the genomic sequence containing the previously known first exon. FIG. 12 shows a comparison between the amino acid sequence of the first exon of Ca(v)1.3 and exon A/B of LV1Ca(v)1.3.

The inserted sequence of one clone containing novel cDNA sequence corresponding to a novel Ca(v)1.3 transcript (clone 8) was subjected to a BLAST alignment analysis against the genomic contig. NT_{—}005986. The analysis showed that the RACE product is composed of three regions. The first region (exon A) corresponds to a portion of the coding region of the cDNA for LV1Ca(v)1.3 and corresponds to nucleotides 1947035-1947104 of the genomic contig NM_{—}005986. This portion encodes the amino acid sequence MFYI and is in the intronic region of HSA275986 mRNA transcription factor gene, which is encoded on the opposite strand.

The second section (exon B) corresponds to a portion of the coding region of the cDNA for LV1Ca(v)1.3 and corresponds to nucleotides 1950569-1950630 of the genomic contig. This portion encodes the amino acid sequence MMEPLFRCRKTSSRLPLILHD (SEQ ID NO:36) and falls within the intronic region of the HSA275986 mRNA transcription factor gene, which is encoded on the opposite strand.

The third section corresponds to the known sequence encoding Ca(v)1.3 protein, begins at exon 2 of the known sequence, and is located on the contig starting at nucleotide 2121014.

A second set of RACE primers was prepared and used to amplify a Jurkat cDNA preparation. The primers used in the second set of reactions were;

| | |
|---|---|
| D-exon2Race2: | |
| 5′CAGGCTCTTCGGATGGGGTTATTGA | (SEQ ID NO:33) |
| G3′ | |
| and | |
| D-exon2Race1: | |
| 5′CCCTGTTTTTTGCTCTTGGCGTATT | (the nested primer; |
| GC3′. | SEQ ID NO:34) |

The PCR was performed using standard procedures and the products electrophoresed. The primary band that was detected was cloned into a TOPO2.1 vector (Invitrogen, Carlsbad, Calif.) and the sequences of two clones (termed D-clone3contig and D-clone2contig) were analyzed. Analysis of the cloned sequence revealed the same start as in the first set of RACE products (described above) with the exception of a single nucleotide that results in a single amino acid change in the predicted polypeptide (compared to the predicted polypeptide identified in the first RACE protocol) that results in a proline instead of a leucine. The start site in the second sequence is predicted to encode an amino acid sequence that begins with NH₃-MFYIMEPLFRCRKTSSRLPLILHDEANY . . . —COOH (SEQ ID NO:35). In addition, the novel exons A and B are spliced to exon 2 that has been identified in other (e.g., neuronal) Ca(v)1.3 sequences.

A BLAST analysis of the cloned sequences was performed using the default parameters and did not identify any other sequences in GenBank with significant homology to the novel (exon 1) portion of the cloned sequences. A CDC-like kinase 2 motif (consensus sequence) was identified using the Scansite program (scansite.mit.edu) in the novel Ca(v)1.3 sequences; MMEPLFRCRKTSSRLPLILHD (SEQ ID NO:36). This site is in novel exon B. The TSS portion of the sequence (in boldface type) represents a consensus site for targets of basophilic serine and threonine kinases, of which CLK2 and PKA are members. This site is therefore a novel phosphorylation site, whose phosphorylation status can be affected to modulate activity of an LVCa(v), e.g., for regulating calcium influx in a non-excitable cell or other cell type expressing an LVCa(v). This site provides LVCa(v) channel forms with a unique activation/sensitivity and/or localization signal related to the activity of, e.g., CLK2 within T lymphocytes. Note that this kinase family is also involved in RNA splicing.

5′ Untranslated regions of LVCa(v)1.3 sequences were also investigated and three related 5′ untranslated regions were identified. The three sequences are shown in FIG. 5D as is a consensus sequence. In general, such sequences are useful, e.g., for detecting expression of an LVCa(v)1.3 RNA molecule or for targeting an LVCa(v)1.3 sequence in the genome or in RNA. In most cases, the sequences used are those that are in common between all three 5′ sequences of LVCa(v)1.3mRNAs.

Identification of a 3′ Terminus of LVCa(v)1.3

To amplify the 3′ end of the lymphocyte-specific sequence, 3′ RACE was performed with the Generacer Kit (Invitrogen) according to manufacturer's recommendations. A set of nested primers was designed targeting exon 44. The following gene-specific primers were used: 3′ Primer 1: 5′CAAGTTCCCACCTCAACAAATGCCAATC3′ (SEQ ID NO:37) and 3′ nested primer: 5′ACAAATGCCAATCTCAATAATGCCAAT3′ (SEQ ID NO:38). The other primers used in the protocol were supplied in the kit (i.e., primers for the 3′ end). A 600 bp fragment was obtained, TOPO cloned, and sequenced. The sequence revealed a stop codon (TAA) at the end of the sequence corresponding to exon 44. Subsequent repeats of the experiment confirmed this result. The sequences for three experiments determining the 3′ untranslated region are shown in FIG. 5C. A consensus sequence for this region is also shown is FIG. 5C (SEQ ID NO:42). Race primers that were designed using sequences corresponding to exons 46 and beyond did not yield any products using Jurkat cDNA. This is consistent with the result conclusion that the primary end of the Ca(v)1.3 gene that is expressed in Jurkat is at the end of exon 44. Finally, the entire gene cDNA sequence was generated by PCR from the beginning of the new exons A/B to the end of exon 44. Primers designed to hybridize to exon 46 or beyond exon 46 did not yield any products, again consistent with the observation that the primary product extends only to the end of exon 44.

One of the newly identified Ca(v)1.3 sequences is shown in FIGS. 6A-6C (SEQ ID NO:17) and is termed LV1Ca(v)1.3. The predicted translation of the sequence is shown in FIG. 6D (SEQ ID NO:21).

Analysis of the Exon Structure of LVCa(v)1.3

In addition, to lacking neuronal exon 1 that is replace by novel exons A/B, and terminating at the end of exon 44, several other features of the Ca(v)1.3 sequences expressed in Jurkat cells were identified.

Exon 12 of the L-type channel gene was deleted in all of the novel splice variants identified in the Jurkat cells. This exon is, in previously identified Ca(v)1.3 sequences (neuronal), located in the cytoplasmic loop between Motifs I and II. The exon 12 sequence is 5′CWWRRRGAAKAGPSGCRRWG3′ (SEQ ID NO:43). Accordingly, LVCa(v)1.3 sequences can be identified, at least in part, by the absence of this sequence and by a novel sequence that is created by the conjoining of exons 11 and 13 (FIGS. 3A and 3B).

Exon 33 is also deleted in certain the novel Ca(v)1.3 cDNAs expressed in non-excitable cells or a novel exon 33 (exon 33a, FIGS. 2A and 2B) is substituted (see infra). This deletion/alteration impacts channel gating since the polypeptide sequence encoded by exon 33 has been implicated in the gating function. The neuronal Ca(v)1.3 exon 33 sequence is 5′PTESENVPVPTATPG3′ (SEQ ID NO:44). Thus, some LVCa(v)1.3 sequences can be identified, at least in part, by the absence of this exon 33 sequence (and the presence of a novel sequence conjoining exons 32 and 34) or usage of the alternate (i.e., non-neuronal) exon 33a sequence.

Another alternatively spliced Ca(v)1.3 sequence was also identified by its expression in Jurkat cells and is termed herein LV2Ca(v)1.3. The cDNA sequence for LV2Ca(v)1.3 (coding region) is shown in FIGS. 8A-C (SEQ ID NO:20) and the predicted amino acid sequence is shown in FIG. 8D (SEQ ID NO:22). LV2Ca(v)1.3 is similar to LV1Ca(v)1.3 except that, as described above, an alternate splice replaces exon 33 with a novel sequence, HYFTDAWNTFDALIVVGSVVDIAITEVN (exon 33a, (SEQ ID NOs:9 and 10). Exon 33a appears to be produced via an alternative splicing event.

As discussed above, changes in exon 33 can affect channel function and gating in lymphocytes since alterations of this region in neuronal Ca(v)1.3 polypeptides have been reported to alter gating properties and this exon is in the S3-S4 linker region within the fourth domain. This region affects the voltage-dependence and activation of voltage-gated calcium channels (VOCCs) (Koschak et al., 2001, J. Biol. Chem. 276:22100-22106; Bourinet et al., 1999, Nature Neurosci. 2:107-415) and is a loop that connects in the extracellular region.

The novel LVCa(v)1.3 cDNA sequences also terminate after exon 44. Thus, LVCa(v)1.3 sequences can be identified, at least in part, by the absence of exons 45-55, which are expressed in neuronal Ca(v)1.3 transcripts.

These data demonstrate that a non-excitable cell type, Jurkat cells, express a novel set of Ca(v)1.3 sequences that have several structural features that distinguish them from other, previously identified Ca(v)1.3 sequences. Notable are changes in the N-terminus that result in a previously unknown start site, a previously unknown pair of exons, alterations in the IV53-IV54 linker (exon 33) that can affect voltage sensitivity, and termination after exon 44. These features suggest a unique functionality for these polypeptides in Jurkat and other cell types. This is consistent with the mechanisms of calcium regulation in non-excitable or minimally excitable cells such as cells of the immune system.

### Example 2

### Expression of LVCa(v)1.3 Channel Polypeptides

To determine whether a Ca(v)1.3 calcium channel is expressed in non-excitable cells, expression of Ca(v)1.3 was examined using immunocytochemical methods. Briefly, expression of Ca(v)1.3 was examined in extracts of T and B cells (Jurkat and DT40 cells, respectively) that were immunoprecipitated with an antibody that recognizes the Ca(v)1.3 sequence (α1D) and was purchased from Alomone (Jerusalem, Israel). The immunoprecipitates were subjected to SDS-PAGE and transferred to PVDF membranes (Immobilon/Millipore, Billerica, Mass.). Immunoblotting was performed with the Cav1.3 antibody according to manufacturer's recommendations.

As predicted from the sequence data, the length of the immunoprecipitated protein was shorter than that detected by immunoprecipitation of protein from neurons. In fact, the length corresponds to the size of a protein that would be produced by termination of the Ca(v)1.3 sequence at the sequence corresponding to exon 44.

These data demonstrate that an Ca(v)1.3 calcium channel is expressed in at least two immune system cell types and confirms the identification of the LVCa(v)1.3 sequence as being shorter than previously identified neuronal sequence.

### Example 3

### Dominant Negative Effect of LVCa(v)1.3 Expression in Jurkat Cells

To further examine the activity of LVCa(v)1.3 sequences in an immune system cell type, a mutant variant of an LVCa(v)1.3 sequences was constructed and expressed in Jurkat cells using the T-Rex system (Invitrogen, Carlsbad, Calif.). This sequence was designed to function as a dominant negative mutant that was predicted to block the effects of endogenous Ca(v)1.3.

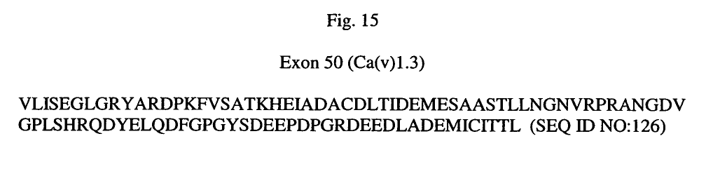

The mutant variant nucleic acid sequence encoded a three-domain form of Ca(v)1.3 that lacked the first transmembrane spanning region. The three-domain form begins with exon 11, skips the next exon (exon 12), and contains the remaining exons, except for exon 45 (which encodes the sequence NH₃-RTRYYETYIR-COOH; SEQ ID NO:45), through the end of exon 50. The amino acid sequence for exon 50 is depicted in FIG. 15. Note that in general, certain LVCa(v)1.3 sequences can be distinguished, at least in part, by their lack of exon 50.

The three-domain variant sequence of Ca(v)1.3 was constructed using RT-PCR methodology. This variant of Ca(v)1.3 was transfected into Jurkat cells using the T-Rex system and the 4TO vector (Invitrogen), and overexpressed. To aid identification of the expressed variant, an N-terminal FLAG tag was included in this vector upstream of the translation start site of the variant sequence. The unique 5′ end was constructed using 5′ RACE (Invitrogen) using the following primer sequences that were designed using the known sequence for neuronal Ca(v)1.3;

| | |
|---|---|
| primer 1: | |
| 5′AGTGTTCAGACTTTCAGCATCAGCCAAAT3′, | (SEQ ID NO:46) |
| and | |
| primer 2: | |
| 5′CAGCATCAGCCAAATTGTCTACAGCGA3′. | (SEQ ID NO:47) |

The 3′ end was verified as corresponding to the 3′ terminus of the neuronal sequence using 3′ RACE PCR (Invitrogen) with the following primers:

| | |
|---|---|
| primer 3: | |
| 5′CGATGACTCGCCCGTTTGCTATGATTC3′ | (SEQ ID NO:48) |
| primer 4: | |
| 5′TGCTATGATTCACGGAGATCTCCA3′ | (SEQ ID NO:49) |

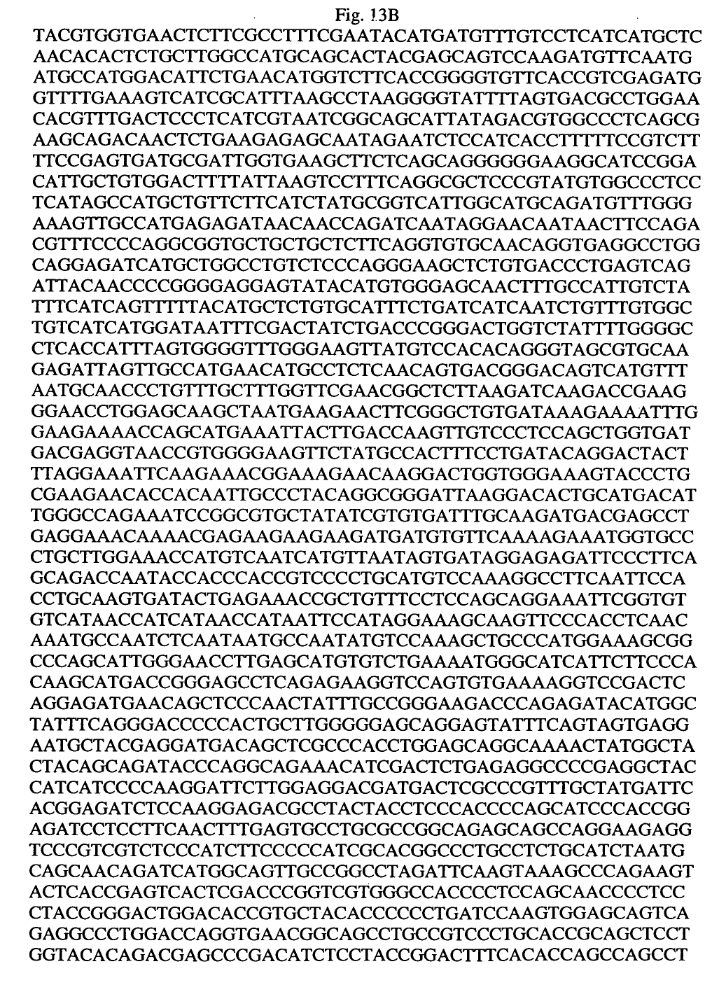

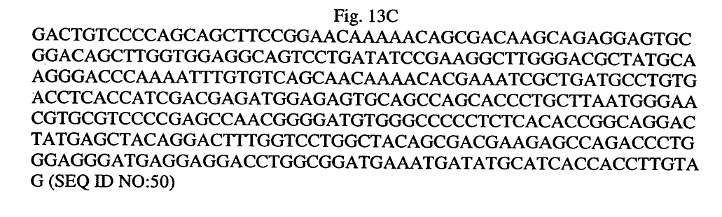

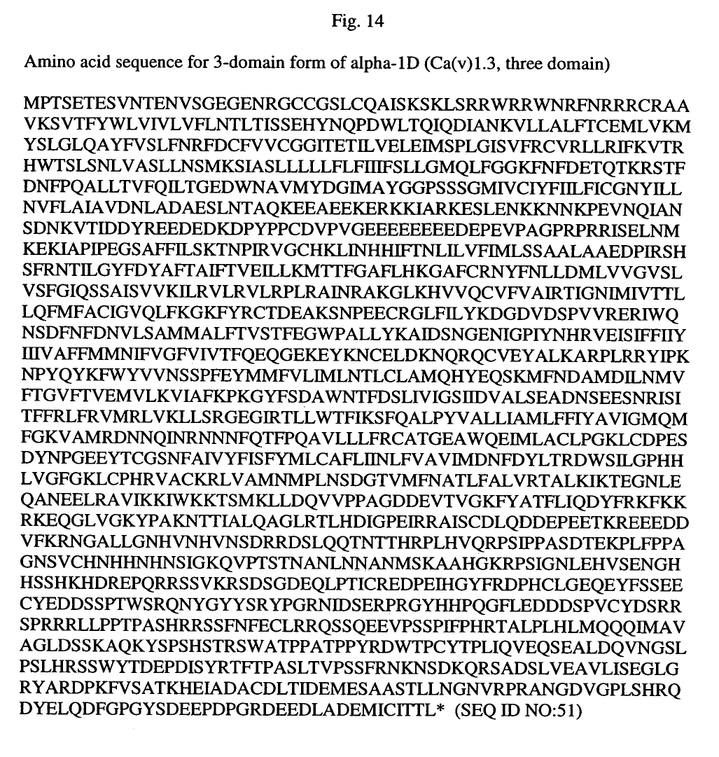

The nucleotide sequence of the three-domain version of Ca(v)1.3 is shown in FIGS. 13A-13C (SEQ ID NO:50). The amino acid sequence is shown in FIG. 14 (SEQ ID NO:51).

The three-domain Ca(v)1.3 was inserted into an expression vector with a CMV promoter such that a chimeric protein that included a FLAG sequence would be expressed. The vector was transfected into Jurkat TRex cells. Cell lines that stably expressed the three-domain Ca(v)1.3 were selected. Two clonal cell lines were analyzed. Overexpression of the three-domain Ca(v)1.3 protein was analyzed by immunoprecipitation and immunoblotting. The three-domain Ca(v)1.3 product was detected using an anti-FLAG2 antibody (Sigma) according to manufacturer's recommendations. Expression of the three-domain Ca(v)1.3 product was detected in both clonal cell lines. Expression was also assayed using a Ca(v)1.3-specific antibody for immunoprecipitation and Western blots.

To assess the effect of expressing the mutant three-domain Ca(v)1.3 on calcium influx in cells, bulk calcium assays were performed using fura-2 (Molecular Probes) ratiometric imaging according to the manufacturer's recommendations. Fura-2 is a calcium-sensitive dye. When excited at short ultra-violet wavelengths (340 nm) the fluorescence of fura-2 increases with increasing calcium concentration, whereas fluorescence decreases with increasing calcium concentration at longer wavelengths (380 nm). By forming a ratio of successive images obtained at each wavelength, a measure of calcium concentration inside the fura-2 loaded cell is obtained.

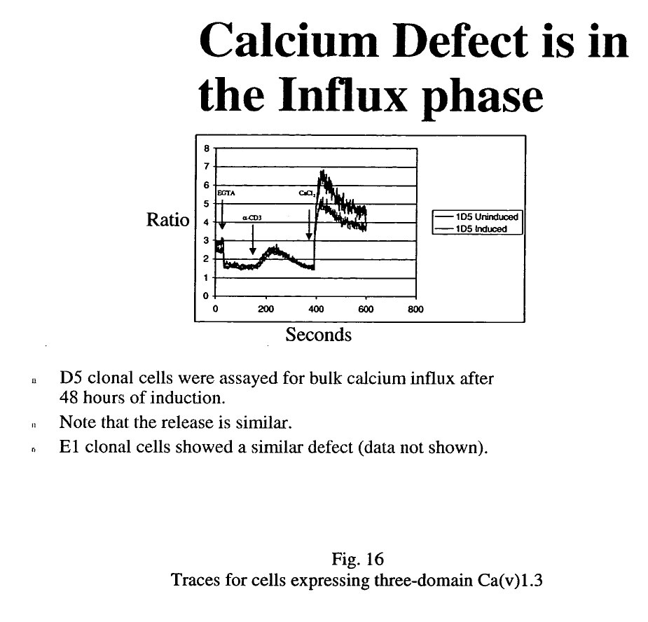

Cells overexpressing the three-domain Ca(v)1.3 were loaded with fura-2 and tested for calcium flux. Cells expressing the three domain Ca(v)1.3 sequence were demonstrated to have inhibited calcium flux compared to cells that did not express the three-domain Ca(v)1.3 sequence (FIG. 16).

These data demonstrate that Ca(v)1.3 sequence expressed in a non-excitable cell type are involved in calcium flux.

### Example 4

### Generation and Analysis of a DT40 Knockout Cell Line

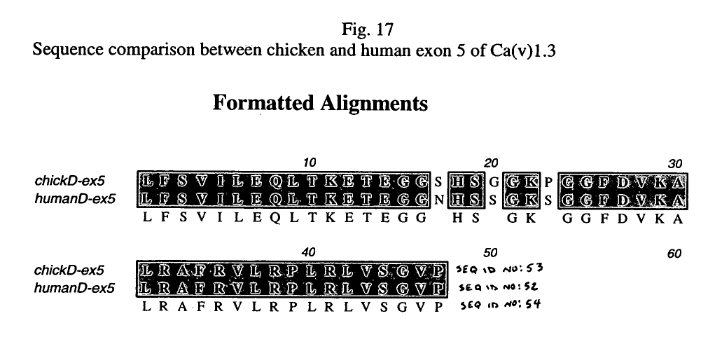

The DT40 chicken (gallus) B cell line has been used extensively to study the involvement of genes in various signaling and growth pathways. Due to its unusually high rate of homologous recombination, a gene of interest can be ablated by the sequential targeting of its alleles in this cell line. The cDNA sequence for the chicken Ca(v)1.3 is known (GenBank accession number AF027602). However, the genomic sequence of chicken Ca(v)1.3 is not known. There is sufficient homology between the human Ca(v)1.3 cDNA sequence and the chicken sequence so that the human sequence can be used as a template for designing primers. Genomic sequence surrounding the chicken sequence that corresponds to the human exon 5 was therefore obtained. Human exon 5 encodes the following amino acid sequence NH₃-LFSVILEQLTKETEGGSHSGGKPGGFDVKALRAFRVLRPLRLVSGVP-COOH (SEQ ID NO:52). FIG. 17 shows an alignment between chicken and human exon 5.

A 3.5 kb intron between exons 4-5 was generated by PCR to form the 5′ arm of the targeting construct. 2.6 kb of genomic sequence that encompasses exon 6 and the intron between exons 6-7 was generated and utilized as the 3′ arm in the targeting construct. The targeting construct contained the B-actin/neo cassette on a pBluescript backbone (Promega). Sequences of this and other cassettes and details regarding targeting are available on the DT40 website: swallow.gsf.de (Buerstadde et al., 2002, Nuc. Acids Res. 30:230-231).

In these experiments, each intron was obtained independently and cloned via TOPO cloning and then sequenced. Thus, primers (shown in Table 1) were designed from exon to exon, so that they would overlap. Once the sequence was obtained, the pieces were assembled by PCR to generate the arms that surround exon 5. All primers were based on the GenBank sequence available for a chicken 1D (Ca(v)1.3) sequence. All primers are denoted 51-3′.

TABLE 1

| | | |
|---|---|---|
| Primers | | |
| for obtaining | | |
| intron between | | |
| exon 4-5 | | |
| Exon 4F | CATATGGATTATTATTACACC | (SEQ ID NO:55) |
| | CCAATG | |
| Exon 5R | CGGAGAGGTCGCAATACACGA | (SEQ ID NO:56) |
| | AAG | |
| Primers | | |
| for obtaining | | |
| intron between | | |
| exons 5-6 | | |
| Exon 5F | AAGCCCTAAGAGCCTTTCGTG | (SEQ ID NO:57) |
| | TATTG | |
| Exon 6R | ACCAAAAGGGCAATATGGAGC | (SEQ ID NO:58) |
| | AG | |
| Primers | | |
| for obtaining | | |
| intron between | | |
| exon 6-7 | | |
| Exon 6F | ⁵⁹) | |
| Exon 7R | TCGTTATGCCTCCATTCGGTC | (SEQ ID NO:60) |
| | CAAC | |

Other alleles of chicken exon 5 of Ca(v)1.3 have been targeted using other drug cassettes described on the website.

Exon 5 was selected for this study because i) it is a 5′ sequence that is not involved in the start site of Ca(v)1.3, i.e., it does not contain exons 1-2 that may be altered, for example, by alternative splicing in lymphocytes; and ii) it is in a highly conserved region of the calcium channel pore that is required for proper functioning.

All PCR experiments were performed as described above except that genomic lysates, prepared as recommended on the website (swallow.gsf.de), were used as the template. Correct targeting was determined by long-range PCR as described on the website.

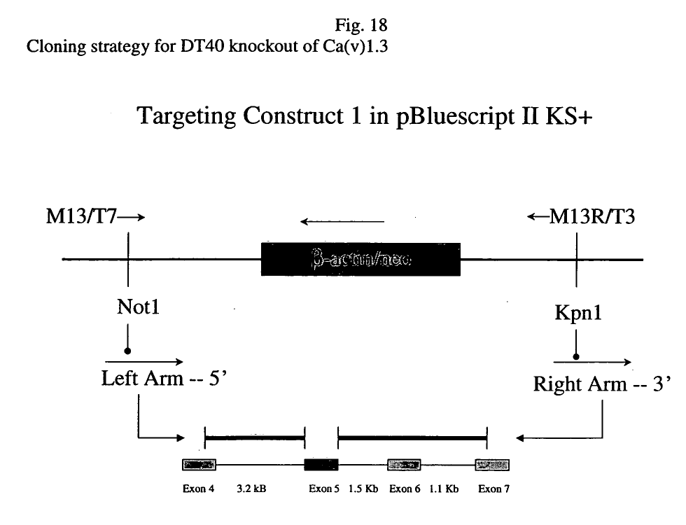

FIG. 18 is a schematic drawing of a targeting construct for DT40. Additional targeting constructs have the same design except that they have a different drug cassette.

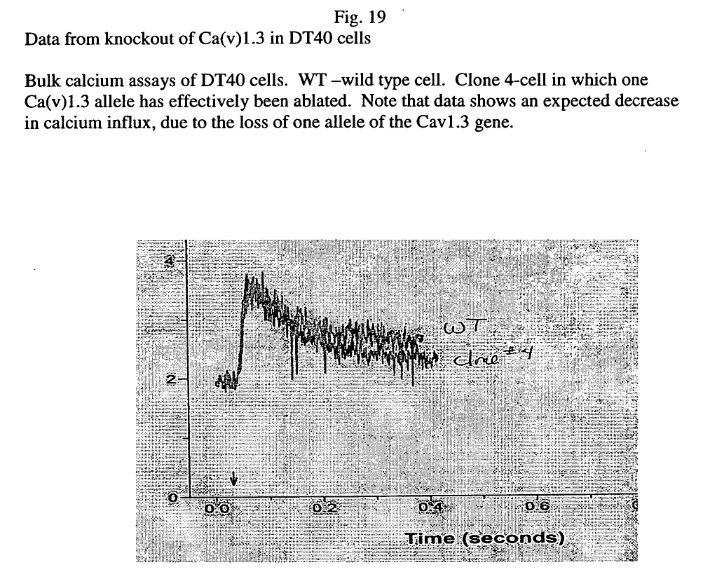

DT40 cells were transfected with the constructs targeting exon 5 and selected for knockout of at least one allele of the chicken sequence corresponding to exon 5. Bulk calcium assays were performed to analyze the effect of loss of an allele on calcium influx using wild type cells or a cell in which on Ca(v)1.3 allele has effectively been ablated (Clone 4 in FIG. 19). FIG. 19 shows the data from such an assay. Cells with a knockout of a sequence corresponding to Ca(v)1.3 exon 5 (Clone 4) showed a decrease in calcium influx compared to cells that were not knocked out. These data demonstrate the expression of a Ca(v)1.3 sequence in an immune system cell is related to the regulation of calcium flux.

Note that these data show an expected decrease in calcium influx, due to the loss of one allele of the Ca(v)1.3 gene. This supports the assertion that the expression of Ca(v)1.3-derived sequence in non-excitable cells serves a function related to calcium flux.

### Example 5

### Identification of a Novel Ca(v)1.1 Channel

A novel Ca(v)1.1 (α1S) expressed nucleic acid sequence was also identified in Jurkat cells. The GenBank accession number for human neuronal Ca(v)1.1 sequence is NM_{—}000069 and the genomic sequence is located on chromosome 1q32. The accession number for the human contig that contains the 1S genomic sequence was originally identified in NT_{—}029862. This contig was replaced with NT_{—}004671. All primers for RT-PCR Ca(v)1.1 sequences were designed based on the cDNA of Genbank accession number NM_{—}000069. The primers used for the 5′ RACE PCR of Ca(v)1.1 in Jurkat cell mRNA were as follows;

| | |
|---|---|
| primer 1: | |
| TGAAGTCCAGCACATTCCAGCCACTG | (SEQ ID NO:61) |
| primer 2: | |
| AGCGTCCTGGTGGAATAAGAAGCCGTAG | (SEQ ID NO:62) |

Ca(v)1.1 sequence having a unique start was identified in mRNA isolated from Jurkat cells. Exons 1 and 2 of neuronal Ca(v)1.1 are replaced in the novel sequence with three new exons as shown in Table 2. This Table shows the locations of LVCa(v)1.1 sequences that were identified in NT_{—}004671 using a BLAST, two-way pairwise BLAST analysis (blast two sequences against each other) using the following default parameters: open gap=5, extension gap=2, gap x_dropoff=50, expect=10.0, work size=11, and filter=on. Table 2 also shows the approximate intron/exon boundaries on the minus strand for the neuronal form of Ca(v)1.1 (from the NM_{—}000069 contig).

TABLE 2

| | | | Intron size |
|---|---|---|---|
| Known start to | Exon location in | | (following |
| Ca(v)1.1 mRNA | contig NM_00467 | Exon size (bp) | listed exon) |
| Neuronal Ca(v)1.3 | | | |
| exons | | | |
| Exon 1 | 12436451 − | 153 bp | 1839 |
| | 12436299 | | |
| Exon 2 | 12434381 − | 107 bp | 3456 |
| | 12434275 | | |
| Exon 3 | 12418134 − | 142 bp | 1761 |
| | 12417993 | | |
| Exon 4 | 12416227 − | 144 bp | 474 |
| | 12416083 | | |
| Novel start in | | | |
| LVCa(v)1.1 mRNA | | | |
| Exon A′ | 12470935 − | 86 bp | |
| | 12470850 | | |
| Exon B′ | 12468065 − | 135 bp | |
| | 12467931 | | |
| Exon 3 | same in neuronal | | |
| | Ca(v)1.1 | | |

Genomic sequence corresponding to the new Ca(v)1.1 sequences listed above were identified in the genomic contig as being generated by unique splicing events within an intronic sequence internal to the known Ca(v)1.1 genomic sequence.

The LVCa(v)1.1 5′ RACE product cDNA sequence is; [AAAAGTCTTTTGCGGCTGCAGCGGGCTTGTAGGTGTCCGGCTTTGCTGGCCCAG CAAGCCTGATAAGCATGAAGCTCTTATCTTTGGTGGCTGTGGTCGGGTGTTTGCT GGTGCCCCCAGCATGAAGCCAACAAGAGTTCTGAAAATATCCGGNGCAAATGCA TCTGTCCACCTTATAGAAACATCAGAGGGCACATTTACAACCAGAATGTATCCCA GAAGGACTGCAACTGCCTGCACGTGGTGGAGCCC]ATGCCAGTGCCTGGCCATGA CGTGGAGGCCTACTGCCTGCTGTGCGAGTGCAGGTACGAGGAGCGCAGCACCAC CACCATCAAGGTCATCATTGTCATCTACCTGTCCGTGGTGGGTGCCCTGTTGCTCT ACATGGCCTTCCTGATGCTGGTGGACCCTCTGATCCGAAAGCCGGATGCATACAC TGAGCAACTGCACAATGAGGAGGAGAATGAGGAGAAGCTGGAGTATTTCTTCCT CATTGTCTTCTCGATTGAAGCCGCCATGAAGATCATTGC (SEQ ID NO:63) The RACE product terminates within exon 3 because of the primer sequence used. The complete sequence includes all of exon 3. The region enclosed in brackets is 5′ untranslated region. The sequence beyond the brackets constitutes the splicing of two novel exons and exon 3. The sequence of the two novel exons (A′ and B′) is ATGCCAGTGCCTGGCCATGACGTGGAGGCCTACTGCCTGCTGTGCGAGTGCAGG TACGAGGAGCGCAGCACCACCACCATCAAGGTCATCATTGTCATCTACCTGTCCG TGGTGGGTGCCCTGTTGCTCTACATGGCCTTCCTGATGCTGGTGGACCCTCTGAT CCGAAAGCCGGATGCATACACTGAGCAACTGCACAATGAGGAGGAGAATGA (SEQ ID NO:26). The final two nucleotides in this sequence form a codon with the first nucleotide of exon 3. The actual coding sequence of the novel amino terminus of LVCa(v)1.1 is MPVPGHDVEAYCLLCECRYEERSTTTIKVIIVIYLSVVGALLLYMAFLMLVDPLIRKP DAYTEQLHNEEENE (SEQ ID NO:27). This sequence replaces the sequence encoded by exons 1 and 2 of the neuronal form of Ca(v)1.1. The complete sequence of exon 3, which follows this sequence is EKLEYFFLIVFSIEAAMKIIAYGFLFHQDAYLRSGWNVLDFTIVFLG (SEQ ID NO:64). The currently known sequence of Ca(v)1.1 (exons 1-3) is as follows (sequence replaced in LVCa(v)1.1 is shown in boldface type and brackets);

| | |
|---|---|
| [MEPSSPQDEGLRKKQPKKPVPEILPRPPRALFCLT | (SEQ ID NO:65) |
| LENPLRKACISIVEWKPFETIILLTIFANCVALAVY | |
| LPMPEDDNNSLNLGL]EKLEYFFLIVFSIEAAMKII | |
| AYGFLFHQDAYLRSGWNVLDFTIVFLG. | |

Using PCR, this sequence was also demonstrated to be contained within a larger cDNA sequence.

The novel amino terminus of an LVCa(v)1.1 sequence contains an oleosin domain. This type of domain is predicted to have lipid binding characteristics. Therefore, this domain may target the LVCa(v)1.1 channel to the endoplasmic reticulum compartment or to other membrane compartments such as the plasma membrane. Therefore, compounds that bind to this domain are useful for disrupting localization (and therefore function) of an LVCa(v)1.1.

The 5′ untranslated region of LVCa(v)1.1 sequence was also identified. Two such sequences (SEQ ID NOs:66 and 67) are shown in FIG. 10E with a consensus sequence (SEQ ID NO:68). In general, and similarly to LVCa(v)1.3 sequence, LVCa(v)1.1 sequence lacks the hinge region (corresponding to exon 33, which is absent or replaced, in LVCa(v)1.3 sequences). In Ca(v)1.1, this IV S3-S4 linker region is amino acids 1200-1231 of the sequence in Genbank accession no. Q13698. The sequence of the absent sequence is SEIDTFLASSGGLYCLGGGCGNVDPDESARIS (SEQ ID NO:69) and is encoded by exons 28-30.

### Example 6

### Differential Tissue Expression of L-Type Calcium Channel mRNAs

To determine the expression pattern of L-type calcium channels in various tissues, RT-PCR was used to examine the expression of twelve L-type channel genes in spleen, brain, and thymus. In each sample, both a specific Ca(v) gene and a GAPDH gene were amplified. The GAPDH gene served as an endogenous control that is an active reference. The data from the GAPDH gene were used to normalize the quantification of mRNA target for the difference in the amount of total RNA used in each reaction. The comparative threshold cycle method (DDCT method; Applied Biosystems) was used to perform quantitative PCR. In the method used, the amount of targeted, normalized to GAPDH reference was measured relative to a calibrator. In general, the most highly expressed gene in a tissue of in a cell type is used as the calibrator and is assigned a value of 1.

The basis for the PCR quantitation was to continuously measure PCR product accumulation using a dual-labeled fluorogenic oligonucleotide probe (TaqMan® probe). This probe was composed of a short (approximately 20-25 bases) oligodeoxynucleotide that was labeled with two different fluorescent dyes. On the 5′ terminus was a reporter dye and on the 3′ terminus was a quenching dye. This oligonucleotide probe sequence is homologous to an internal target sequence present in the PCR amplicon. When the probe is intact, energy transfer occurs between the two fluorophors and emission from the reporter is quenched by the quencher. During the extension phase of PCR, the probe is cleaved by 5′ nuclease activity of Taq polymerase, thereby releasing the reporter from the oligonucleotide-quencher and producing an increase in reporter emission intensity. Software was used to examine the fluorescence intensity of reporter and quencher dyes and to calculate the increase in normalized reporter emission intensity over the course of the amplification. The results were then plotted versus time, represented by cycle number, to produce a continuous measure of PCR amplification. To provide precise quantification of initial target in each PCR reaction, the amplification plot was examined at a point during the early log phase of product accumulation.

As stated above, primer sets for this method should be exclusively specific to the analyzed gene.

Because the alpha-1 gene family shares a strong sequence homology, an alignment study was performed for each alpha-1 channel cDNA nucleotide sequence. The purpose of this analysis was to define for each gene a unique sequence area for designing primer sets.

Table 3 summarizes region (assigning the A of ATG of the open reading frame of the sequence as nucleotide 1) and specific sequence of primer design. For some genes (alpha-1 B and D) primer optimal sequences were difficult to identify. Therefore, two sets of primers were design and tested for these genes.

TABLE 3

| | | | |
|---|---|---|---|
| | Nt | | |
| | from | | |
| Primer | start | Sequence | Tm |

| | | | | |
|---|---|---|---|---|
| For Alpha 1B1 | 5329 | CCGGCTGTGCTCCGAG | (SEQ ID NO:70) | 56 |
| Rev Alpha 1B1 | 5403 | TTGCTGAGGGAGGTGGAACT | (SEQ ID NO:71) | 62 |
| Probe Alpha | | TCAGCCCCCAGACCCGAACACTATT | (SEQ ID NO:72) | 78 |
| 1B1 | | | | |
| For Alpha 1B2 | 2728 | GCAGACAATCAGCGGAACGT | (SEQ ID NO:73) | 62 |
| Rev Alpha 1B | 22881 | CGTCAGCATCACTGGGATATGTA | (SEQ ID NO:74) | 68 |
| Probe Alpha | | AGCCCGGGTTTTCCTTCGACAGAA | (SEQ ID NO:75) | 74 |
| 1B2 | | | | |
| For Alpha 1C | 5646 | TGACGAAAATCGGCAACTGA | (SEQ ID NO:76) | 58 |
| Rev Alpha 1C | 5700 | GGAAACCCCTCTTCGGAGAT | (SEQ ID NO:77) | 62 |
| Probe Alpha | | CCCAGAGGAGGACAAGAGGGACATCC | (SEQ ID NO:78) | 84 |
| 1C | | | | |
| For Alpha 1D | 5674 | CCAGGCAGAAACATCGACTCT | (SEQ ID NO:79) | 64 |
| Rev Alpha 1D1 | 5748 | CGAGTCATCGTCCTCCAAGAA | (SEQ ID NO:80) | 64 |
| Probe Alpha | | CCCGAGGCTACCATCATCCCCAA | (SEQ ID NO:81) | 74 |
| 1D1 | | | | |
| For Alpha 1D2 | 5182 | CACCGTCCCCTGCATGTC | (SEQ ID NO:82) | 60 |
| Rev Alpha 1D2 | 5264 | TGTTTCCTCCAGCAGGAAATTC | (SEQ ID NO:83) | 64 |
| Probe Alpha | | AAGGCCTTCAATTCCACCTGCAAGTGAT | (SEQ ID NO:84) | 82 |
| 1D2 | | | | |
| For Alpha 1E | 2724 | GACAGAAGGCAAGGAGTCCTCTT | (SEQ ID NO:85) | 70 |
| Rev Alpha 1E | 2808 | TCAGTGGGCATGGCTTCAT | (SEQ ID NO:86) | 58 |
| Probe Alpha | | TCTGCCAGCCAGGAACGCAGTCT | (SEQ ID NO:87) | 74 |
| 1E | | | | |
| For Alpha 1F | 5187 | CAAAGGGCAAAACAAGCAAGA | (SEQ ID NO:88) | 60 |
| Rev Alpha 1F | 5260 | TCCCTGCCTGCTCATCTAGGT | (SEQ ID NO:89) | 66 |
| Probe Alpha | | AGGATGAGGAAGTCCCTGATCGGCTTTC | (SEQ ID NO:90) | 86 |
| 1F | | | | |
| For Alpha 1G | 1764 | AGGTGTATCCCACCGTGCA | (SEQ ID NO:91) | 60 |
| Rev Alpha 1G | 1836 | GCAGCCACCTCTACTAGTGCCT | (SEQ ID NO:92) | 70 |
| Probe Alpha | | CCTCCACCGGAGACGCTGAAGG | (SEQ ID NO:93) | 74 |
| 1G | | | | |
| For Alpha 1H | 6339 | CCCCAGCTTTGCCTTTGAG | (SEQ ID NO:94) | 60 |
| Rev Alpha 1H | 6454 | GCACTATGGCCCCTGAAGAG | (SEQ ID NO:95) | 64 |
| Probe Alpha | | TTCTTGGACGGTAGCCACAGTGTGACC | (SEQ ID NO:96) | 84 |
| 1H | | | | |
| For Alpha 1I | 5697 | GGGAGACCTGGGCGAATG | (SEQ ID NO:97) | 72 |
| Rev Alpha 1I | 5774 | ATCTCACACAGGAAGTTCTCTGGAT | (SEQ ID NO:98) | 60 |
| Probe Alpha | | TCTTCCCCTTGTCCTCTACGGCCG | (SEQ ID NO:99) | 78 |
| 1I | | | | |
| For Alpha 1S | 2059 | TCCAAGGGTCTCCCAGACAA | (SEQ ID NO:100) | 62 |
| Rev Alpha 1S | 2129 | TTGGGTTTCTGCTCCAGCTT | (SEQ ID NO:101) | 60 |
| Probe Alpha | | CAGAAGAGGAGAAGTCAACGATGGCCA | (SEQ ID NO:102) | 82 |
| 1S | | | | |

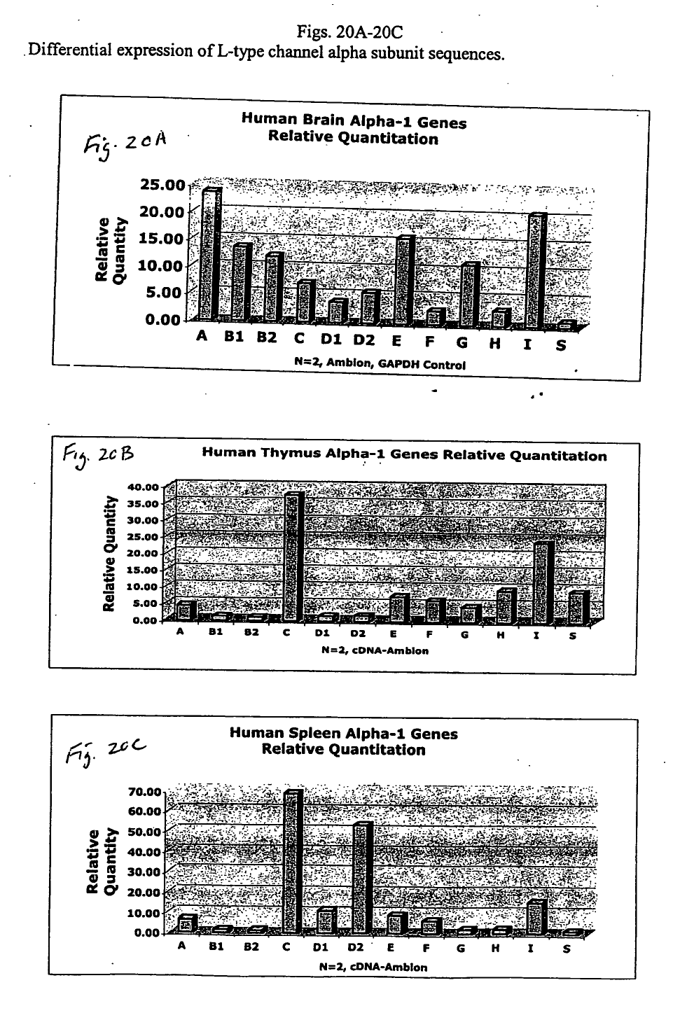

The expression data from these experiments are shown in FIGS. 20A-20C). The X-axis of these figures represents the targeted gene based on the specificity of the primer/probe sets used for the amplification reactions. The Y axis represents a relative quantification of expression of each gene.

FIG. 20A shows the relative expression pattern of L-type channel mRNA in brain. Several genes show prominent expression, including Ca(v)2.1 (α1A), Ca(v)2.2 (α1B), Ca(v)2.3(α1E), and Ca(v)3.3 (α1I). The expression pattern in thymus was surprisingly different. In general, there was low expression of A and B while Ca(v)1.2 had the greatest levels of expression and α1I also demonstrated relatively high levels of expression (FIG. 20B). The most prominent expression in spleen was of Ca(v)1.2 and Ca(v)1.3 genes (FIG. 20C).

The experiments demonstrate that each tissue examined has a unique differential pattern of voltage operated calcium channel gene (VDCC genes) that is distinct from the expression pattern in brain.

These data also indicate that the Ca(v)1.2 (α1C) gene in thymus and the Ca(v)1.2 and Ca(v)1.3 (α1D) genes in spleen are the most highly expressed genes in this family. This surprising result is important because it indicates that these more highly expressed genes may have greater functionality in these respective cell types. Furthermore, as described herein, it has been found that T cells have alternatively spliced (e.g., compared to neurons) Ca(v) sequences. Alternatively spliced sequences (variants) derived from the genes that are most highly expressed in a specific cell type are targets for identifying drugs that modulate calcium metabolism in a cell type expressing the alternatively spliced sequence. The more highly expressed variants are particularly likely candidates as targets for developing drugs for modulating calcium flux in the specific cell type.

### Example 7

### RNAi Inhibition of Ca(v) Expression in Jurkat Cells

To demonstrate that RNAi methodology can be used to useful for knock down expression of L-type channel polypeptides in a non-excitable cell type, an siRNA was designed that had a high degree of sequence identity amongst all four L-type genes; the sequence was an exact match for 1S, 1F, and 1C, and differed from 1D at one base pair as noted below.

| | | |
|---|---|---|
| AACTGTGAGCTGGACAAGAA | consensus | (SEQ ID NO:103) |
| AACTGTGAGCTGGACAAAAA | difference in | (SEQ ID NO:104) |
| | 1D sequence | |

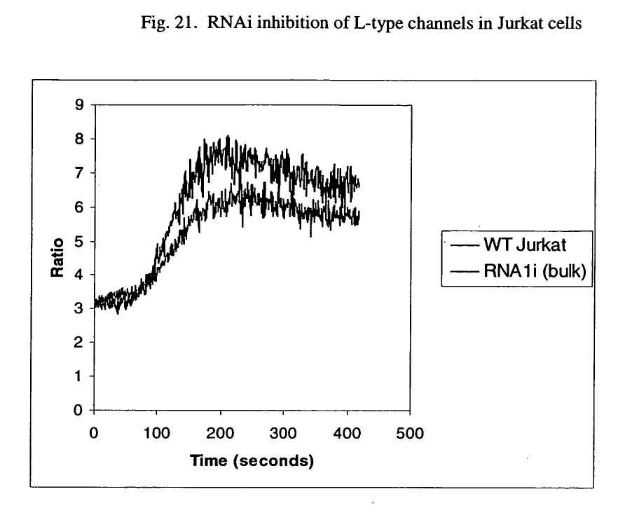

This consensus sequence was used in the design of a sequence that would express an siRNA for stable expression of a hairpin loop containing the consensus sequence. The sequence was cloned into the psiRNA-hHlzeo vector (Invitrogen, San Diego, Calif.), the sequence was verified, and Jurkat cells were transfected with it. To select clones that were positive for expression of the hairpin, 400 micrograms/ml of zeocin was added 24 hours after transfection. A pool of zeocin-resistant clones revealed a decrease in DM-BODIPY binding (Molecular Probes), which was used as a marker for L-type gene expression. These clones grew more slowly and were lost in the bulk culture over time (3 week period). Analysis of this pool of clones revealed a marked decrease in calcium flux, demonstrating the central role of L-type channels in calcium influx in Jurkat cells (FIG. 21). Independent, individual clones were also generated in parallel. These clones showed a marked decrease in DM-BODIPY binding (an approximately 90% reduction) and grew very slowly. Thus, loss of L-type channel expression also correlates with decreased proliferation of Jurkat T cells.

These data demonstrate the association of L-type channels with calcium flux and cellular proliferation in a non-excitable cell type. In addition, these data demonstrate that methods related to the use of siRNA can be used to inhibit expression and activity of L-type channels. Components that specifically target calcium channels expressed in specific all types or that are more highly expressed in certain cell types are useful for selective modulation of the targeted cell type, for example, by inhibiting proliferation of that cell type.

### Example 8

### Identification of Compounds that Modulate LVCa(v)-Containing Channels Using a Cell Proliferation Assay

Assay of cell proliferation is method that can be used to measure the health of cell. During proliferation, DNA is replicated before the cell divides. This close association between DNA synthesis and cell doubling means that monitoring DNA synthesis provides an indirect method for assessing cell proliferation.

Labeled DNA precursors are added to a cell culture, and the precursors are incorporated only into the DNA of cells that are in S phase of the cell cycle. For example, the thymidine analogue, 5-bromo-2′-deoxy-uridine (BrdU), is incorporated into cellular DNA in place of thymidine. Following its incorporation, BrdU can be detected by quantitative cellular enzyme immunoassay using monoclonal antibodies directed against BrdU. Quantification can be performed using commercially available kits (e.g., Roche Cell Proliferation ELISA, BrdU chemiluminescence Cat. No. 1 669 915) that are based on the measurement of BrdU incorporation during DNA synthesis. For example, the assay is performed in 96 well plates that are seeded with cultured cells of interest (e.g., Jurkat cells or HEK293). Briefly, after a period of incubation, e.g., 24 to 120 hours, in the presence or absence of the test compound (e.g., a mitogen, growth factor, cytokine, drugs, or other compound being test for its ability to modulate calcium flux), 10 mM of BrdU is added to the culture medium and cells are incubated for an additional 2 to 24 hours at 37° C. At the end of the labeling period, the labeling medium is removed (generally after centrifugation of the cell cultures) and cells are dried (e.g., for 1 hour at 60° C. or 15 minutes with a hair dryer). Following fixation and a denaturation step, the cells are incubated (e.g., for 90 to 120 minutes) with peroxidase-labeled antibodies directed against BrdU. After washing three times, substrates for detecting the antibodies are added (e.g., luminol and 4-iodophenol). The reaction is quantified by measuring chemiluminescence using a luminometer.

A decrease in cell proliferation indicates that the compound tested inhibited cell proliferation and may be a compound that inhibits calcium flux in the cell. In some cases, the assay is performed using different cell types, e.g., an excitable cell and a non-excitable cell. Alternatively, the assay can be performed in a cell expressing an excitable L-type channel protein (e.g., a neuronal Ca(v) polypeptide) and in a cell expressing a non-excitable L-type channel (e.g., an LVCa(v) polypeptide). Compounds that inhibit cell proliferation in a non-excitable cell to a greater extent than in an excitable cell, or in a cell expressing a LVCa(v) polypeptide compared to a neuronal Ca(v) polypeptide, are candidate compounds for modulation (e.g., inhibition) of calcium flux in non-excitable cell types (e.g., in cell types that express one or more non-voltage gated alpha channel proteins). Cell proliferation assays can also be used to test the effect of a previously identified candidate modulator of a calcium channel on proliferation of a particular cell type.

Similarly, cell viability in the presence and absence of a test compound can be used to evaluate a test compound. Methods for measuring cell viability are known in the art.

### Example 9

### Identification of Compounds that Modulate LVCa(v)-Containing Channels Using an NFAT Assay

In addition to measuring cellular proliferation other indirect methods can be used that assay compounds for their ability to modulate cellular processes known to be affected by calcium metabolism. For example, assay of NFAT (nuclear factor of activated T cells, a transcription factor regulating IL-2) expression or activity is method that can be used to indirectly assess whether a compound modulates calcium flux in a cell.

For example, Jurkat cells are stably cotransfected using electroporation with an NFAT luciferase reporter construct (NFAT-Luciferase, 10 μg) and 1 μg pCS2-(n)-β-gal. Clones are selected in RPMI containing 0.5 mg/ml Hygromycin B. After incubation with test compounds or DMSO control solution, the selected clones are activated with 1 μg/ml of anti-CD3 antibody for 18 hours. Luciferase and β-galactosidase assays are performed on total cell lysates using methods known in the art and measured on a luminometer. For each compound and control, the luciferase activity is normalized to the β-galactosidase activity.

Compounds that inhibit NFAT expression are candidate compounds for inhibition of calcium flux. In some cases, experiments are performed using an excitable cell type and a non-excitable cell type, or in cells expressing, e.g., a neuronal alpha L-type subunit and cells expressing an LVCa(v). Compounds that preferentially inhibit NFAT expression in non-excitable cells or in those cells expressing the LVCa(v) subunit are candidate compounds for preferentially inhibiting calcium flux in via an LVCa(v) subunit. Such compounds are candidates for preferentially inhibiting calcium flux in a non-excitable cell as compared to an excitable or other cell type that, e.g., does not express significant amounts of an LVCa(v) relative to other types of calcium channels.

### Example 10

### Identification of Compounds that Modulate LVCa(v)-Containing Channels Using Patch Clamp Methodology

In general, single channel or whole cell patch clamp methods can be used to examine the effects of a compound on a channel that mediates Icrac. In such experiments, a baseline measurement is established for a patched channel or cell. Then a compound to be tested (e.g., a test compound) is added to the solution in the patch pipette and the effect of the compound on Icrac is measured. A compound that modulates Icrac (e.g., inhibits or increases) is a compound that is useful in the invention for modulating such currents. An example of such an experiment is described below.

Electrophysiology

For patch clamp experiments, Jurkat T cells are grown on glass coverslips, transferred to a recording chamber and kept in a standard modified Ringer's solution of the following composition (in mM); NaCl 145, KCl 2.8, CsCl 10, CaCl₂ 10, MgCl₂ 2, glucose 10, HEPES.NaOH 10, pH 7.2. Compounds of interest (e.g., compounds that are being tested for their ability to modulate activity of a Ca(v) polypeptide) are dissolved in the standard extracellular solution at appropriate concentrations. The standard intracellular pipette-filling solution contains (in mM); Cs-glutamate 145, NaCl 8, MgCl₂ 1, ATP 0.5, GTP 0.3, pH 7.2 adjusted with CsOH. Except for experiments using compounds such as fura-2, the internal solution is supplemented with a mixture of 10 mM Cs-BAPTA and 4.3-5.3 mM CaCl₂ to buffer [Ca²⁺ ]i to resting levels of 100-150 nM and to avoid spontaneous activation of I_{CRAC}.

Patch clamp experiments are performed in the tight-seal whole-cell configuration at 21-25° C. High-resolution current recordings are acquired by a computer-based patch clamp amplifier system (EPC-9, HEKA, Lambrecht, Germany). Sylgard®-coated patch pipettes have resistances between 2-4 MΩ after filling with the standard intracellular solution. Immediately following establishment of the whole-cell configuration, voltage ramps of 50 ms duration spanning the voltage range of −100 to +100 mV are delivered from a holding potential of 0 mV at a rate of 0.5 Hz over a period of 300 to 400 seconds. All voltages are corrected for a liquid junction potential of 10 mV between external and internal solutions. Currents are filtered at 2.3 kHz and digitized at 100 μs intervals. Capacitive currents and series resistance are determined and corrected before each voltage ramp using the automatic capacitance compensation of the EPC-9. For analysis, the very first ramps before activation of I_{CRAC} (usually 1 to 3) are digitally filtered at 2 kHz, pooled and used for leak-subtraction of all subsequent current records. The low-resolution temporal development of inward currents is extracted from the leak-corrected individual ramp current records by measuring the current amplitude at −80 mV or a voltage of choice.

Calcium Measurements

The cytosolic calcium concentration of individual patch clamped channels or intact cells is monitored at a rate of 5 Hz with a photomultiplier-based system using a monochromatic light source tuned to excite fura-2 fluorescence at 360 and 390 nm for 20 ms each. Emission is detected at 450-550 nm with a photomultiplier whose analog signals are sampled and processed by the X-Chart software package (HEKA, Lambrecht, Germany). Fluorescence ratios are translated into free intracellular calcium concentration based on calibration parameters derived from patch clamp experiments with calibrated calcium concentrations. In patch clamp experiments, fura-2 is added to the standard intracellular solution at 100 μM. Ester loading of intact cells is performed by incubating cells for 45-60 minutes in a modified Ringer's solution (1 mM extracellular calcium) supplemented with 5 μM fura-2-AM. In all experiments monitoring intracellular Ca²⁺, the external calcium concentration is 1 or 2 mM. Local perfusion of individual cells with carbachol is achieved through a wide-tipped, pressure-controlled application pipette (3 μm diameter) placed at a distance of 30 μm from the cell under investigation.

An alteration (i.e., increase or decrease) in I_{CRAC} indicates that the compound tested is a modulator of I_{CRAC}. In the case of a single channel experiment, a change indicates that the compound can modulate activity of that type of channel. Similar experiments can be used to demonstrate that a tested compound affects certain channels and not others. These methods can also be used in other cell types, including those containing recombinant channel subunits.

### Example 11

### Expression of L-Type Calcium Channels in T Cells

To demonstrate expression of L-type calcium channels in non-excitable cells, and to characterize the L-type channels in these cell types, various T cell leukemia cell lines were examined for their ability to bind a dihydropyridine derivative.

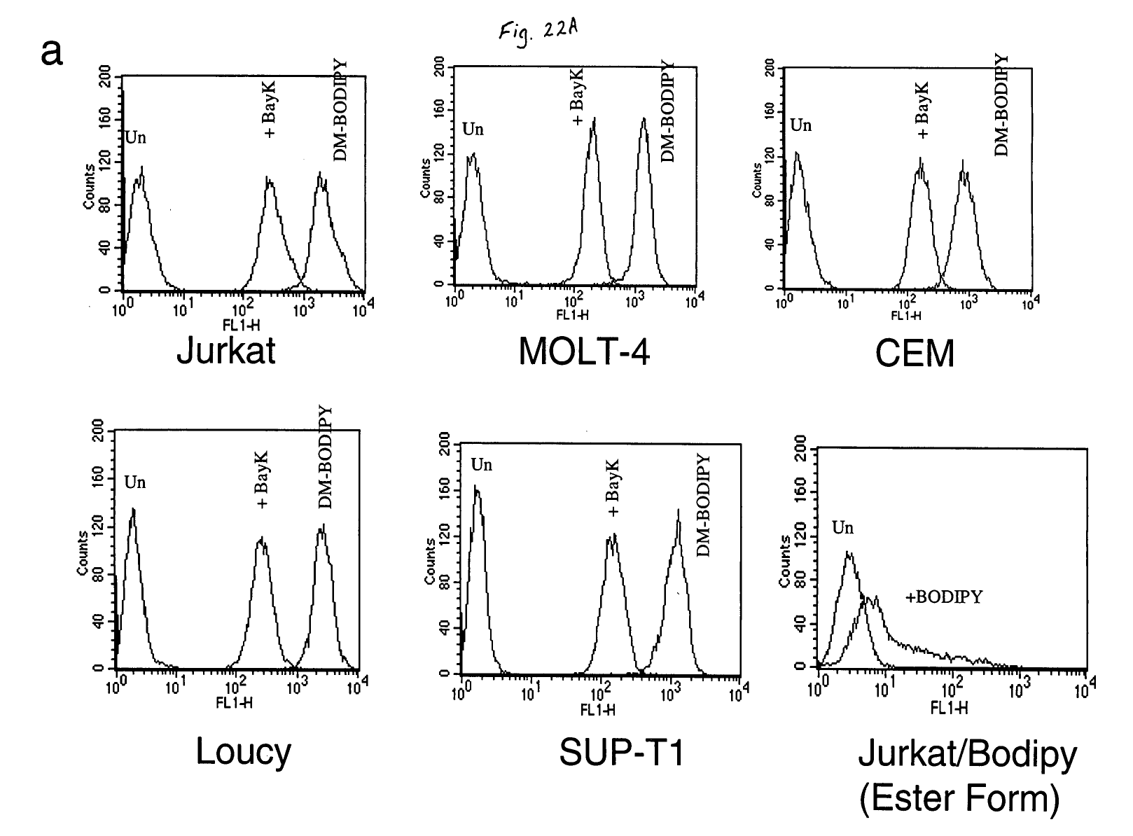

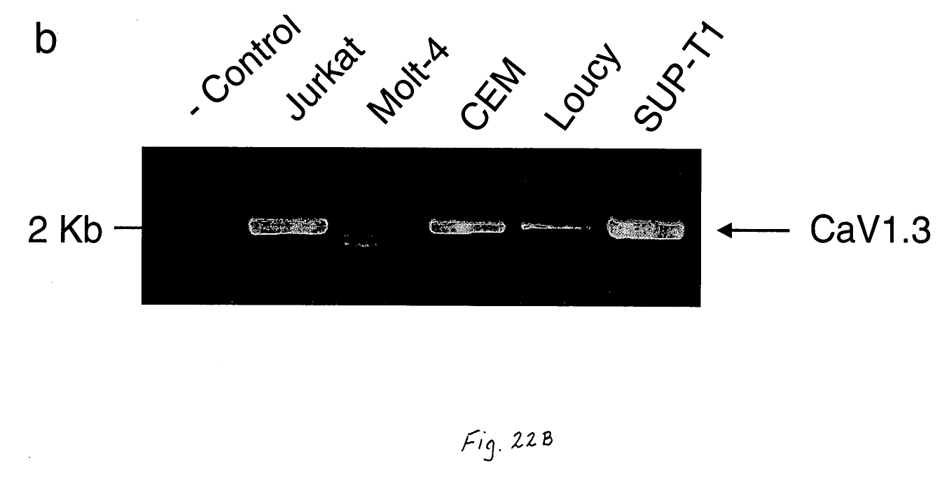

To ascertain the levels of L-type channel expression at the cell surface, cells were stained with an ester form of DM-BODIPY (Molecular Probes, Inc., Eugene, Oreg., catalog no. D-2183) at final concentration of 1 μM in PBS (phosphate buffered saline). Competition of DM-BODIPY binding was performed with a ten-fold excess (S)-(−)-Bay K8644 (Sigma-Aldrich). Analysis was performed using flow cytometry (Becton Dickinson). FIG. 22A illustrates the results of the DM-BODIPY binding experiments. The panels labeled “Jurkat,” “MOLT-4,” “CEM,” “Loucy,” and “SUP-T1” each show a flow cytometry trace of cells from the named cell lines that were unstained (Un), cells stained with DM-BODIPY, and cells treated with an excess concentration of (S)-(−)-Bay K8644, a compound that competes with DM-BODIPY at the same binding site on L-type calcium channels (+BayK). The specificity of DM-BODIPY binding for L-type calcium channels was demonstrated by staining cells with DM-BODIPY in the presence of BayK.

In all of the cell lines tested, the amount of DM-BODIPY binding was reduced, demonstrating the presence of L-type channels that can bind BayK on all of these non-excitable cell lines. Due to the hydrophobic nature of the BODIPY dye itself, there is background binding in the range similar to that competed by the BayK8644 (panel labeled Jurkat/Bodipy).

These experiments were performed on five different T cell lines, all of which are derived from T-ALL (acute lymphoblastic leukemia) patients and were obtained from the American Type Culture Collection (ATCC). The origins of these cell lines are as follows; Jurkat, T lymphoblast cell line, acute T cell leukemia from 14-year-old male; MOLT-4, leukemia cell line, acute lymphoblastic leukemia from 19 year old male; CEM, acute lymphoblastic leukemia, 4 year-old patient; SUP-T1, lymphoblastic leukemia, 8 year-old male patient; and Loucy, T-ALL, from 38 year old female. The ATCC reference numbers for the cells lines are TIB-152 (Jurkat); CCL-119 (CEM); CRL-1582 (MOLT-4); CRL-2629 (Loucy); CRL-1942 (SUP-T1).

All five cell lines demonstrated the presence of DM-BODIPY binding that was competed by BayK8644. This illustrates the presence of L-type channels on a variety of non-excitable cells.

To examine whether transcripts corresponding to Ca(v)1.3 were expressed in these cell types, RT-PCR was performed using primers that specifically amplified Ca(v)1.3 sequences. The D-specific primers used to amplify Ca(v)1.3 were, Forward 1=CAGGCGAAGACTGGAATGCTGTGATG (SEQ ID NO:105); Forward 2=ATGCTGTGATGTACGATGGCATCATG (SEQ ID NO:106); Reverse 1=ATGCTGTGATGTACGATGGCATCATG (SEQ ID NO:107); Reverse 2=TAGATGAAGAACAGCATGGCTATGA (SEQ ID NO:108).

The results of these experiments are shown in FIG. 22B. Ca(v)1.3 was detected in all of the tested cell lines. The MOLT-4 cell line displayed three bands while Jurkat, CEM, Loucy, and SUP-T1 did not display more than one distinct band. This does not preclude the presence of multiple bands that can be obtained from these cell lines. The presence of multiple bands indicates the presence of splice variants in the region corresponding to the amplified sequences.

These data demonstrate the widespread expression of L-type channels in various T cells as well as the as the presence of Cav1.3 within these cell lines.

### Example 12

### Characterization of L-type Channel Calcium Currents in Jurkat T Cells

Jurkat T cells have native L-type currents that are specifically enhanced by (S)-(−)-Bay K8644 (an agonist of L-type currents).

In these patch clamp experiments, Jurkat cells that were grown in suspension cultures were washed once with standard external solution and plated directly onto the experimental bath chamber. Cells were kept in a standard modified Ringer's solution of the following composition (in mM): NaCl 140, KCl 2.8, CaCl₂ 10, MgCl₂ 2, glucose 10, Hepes.NaOH 10, pH 7.2. Intracellular pipette-filling solutions for voltage-gated L-type channels contained (in mM): NaCl 140, KCl 2.8, CaCl₂ 10, MgCl₂ 2, glucose 10, Hepes.NaOH 10, pH 7.2. In some experiments, the above solution was replaced by an otherwise identical extracellular solution in which 10 μM Bay K was added to the bath. Intracellular pipette-filling solutions for voltage-gated L-type channels in some cases contained (in mM): Cs-glutamate 120, NaCl 8, MgCl₂ 1, MgATP 2, NaGTP 0.3, Cs-BAPTA 10, Hepes-CsOH 10, pH 7.2. Patch clamp experiments were performed in the tight-seal whole-cell configuration at 24±2° C. High-resolution current recordings were acquired by a computer-based patch clamp amplifier system (EPC-9, HEKA, Lambrecht, Germany). Patch pipettes had resistances between 2-4 MΩ after filling with the standard intracellular solution. For L-type current measurements the holding potential was −80 mV and the duration of the ramp was 100 ms for optimal L-type current size. All voltages were corrected for a liquid junction potential of 10 mV between external and internal solutions. Currents were filtered at 2.3 kHz and digitized at 100 μs intervals. Capacitive currents and series resistance were determined and corrected before each voltage ramp using the automatic capacitance compensation of the EPC-9. For I_{CRAC} analysis, the very first ramps prior to current activation were digitally filtered at 2 kHz, pooled and used for leak-subtraction of all subsequent current records. The low-resolution temporal development of currents at a given potential was extracted from the leak-corrected individual ramp current records by measuring the current amplitudes at voltages of −80 mV or +80 mV, respectively. Similarly, one of the first ramps could be used as leak-subtraction for L-type currents, as the currents developed over a time course of 6-8 seconds, indicating recovery from inactivation. Where applicable, statistical errors of averaged data are given as means±S.E.M. with n determinations and statistical significance was assessed by Student's t-test.

For measurements of the L-type current with barium as the charge carrier, whole cell voltage-clamp recordings were performed using an Axopatch 200 amplifier (Axon Instruments) in a bath consisting of 135 mM choline Cl, 10 mM HEPES, 1 mM MgCl₂ and 20 mM BaCl₂, adjusted to a pH of 7.2 with CsOH. The internal solution consisted of 135 mM CsCl, 10 mM HEPES, 1 mM EGTA, 1 mM EDTA, and 4 mM Mg-ATP, adjusted to a pH of 7.2 with CsOH. Pclamp 8 software was used for data acquisition (Axon Instruments). For square pulse protocols, data were filtered at 2 kHz and sampled at 10 kHz. Patch electrodes were 3.8-4.4 MΩ when filled with internal solution. Leak subtraction was performed online using a P/-4 protocol. 75% series resistance compensation was used with a 10 μs lag. Statistical references indicate the mean±s.e. After forming the whole-cell patch, cells were lifted off the bottom of the dish and placed in front of an array of perfusion tubes made of 250 μm internal diameter quartz tubing (Polymicro Technologies, Phoenix, Ariz.) connected by Teflon tubing to glass reservoirs. External solutions were exchanged in less than 1 second by moving the cell between continuously flowing solutions from the perfusion tubes. Bay K 8644 and phorbol 12-myristate 13-acetate (PMA) were obtained from Sigma. BayK-8644 was stored at 10 mM stock concentration in DMSO.

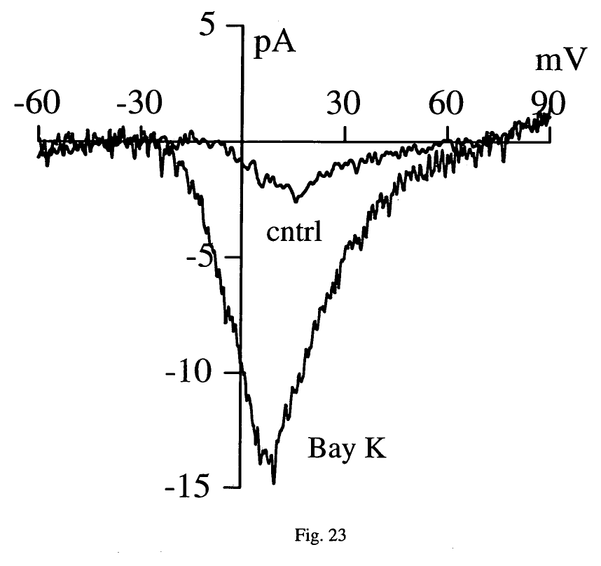

Examples of I/V curves performed in the presence of calcium as the charge carrier are shown in FIG. 23. The I/V curve labeled “cntrl” represents control Jurkat cells that were tested without agonist and the I/V curve labeled “BayK” represents Jurkat cells tested in the presence of 10 μM BayK8644. Control cells had an average of 8 ramps and had 0.44 pA/pF+/−0.1 pA/pF (n=6, +/−s.e.m.) average current densities. Six of fourteen control cells tested had discernible DHPR (L-type calcium channel) currents. BayK-treated cells had an average of 33 ramps and had 1+/−0.3 pA/pF (n=5+/−s.e.m.) average current densities. Five of eleven cells tested in the presence of BayK8644 had discernible DHPR currents. Thus using calcium as the charge carrier, FIG. 23 illustrates that the native current is small but is highly characteristic of L-type calcium channel. The Jurkat L-type current has a reversal potential of about 0 mV and the peak amplitude is smaller (about 3-5 pA) than that seen in excitable cells. In the presence of the L-type agonist, (s)-(−)-Bay K8644 (BayK) augments the current signature; the peak current increases five-fold, thus revealing an unambiguous L-type current. It was also observed that the effect of the BayK agonist was reversible when washed out. In addition, better conductance was observed when barium (e.g., 20 mM) was used as the charge carrier. Furthermore, in Jurkat cells that were pre-treated with PMA (phorbol 12-myristate 13-acetate, an activator of protein kinase C (PKC)), the L-type current amplitude is significantly increased. Therefore, the L-type channels in T cells (e.g., Jurkat cells) are functionally sensitive to PKC-dependent activation pathways.

These data demonstrate that a “non-excitable” cell type (e.g., Jurkat T cells) have L-type voltage-sensitive calcium channels that can conduct calcium currents and are therefore functional channels. These data also illustrate a method of identifying the presence and activity of L-type channels in non-excitable cells. In general, L-type channels identified in non-excitable cells (e.g., T cells) have been identified as “non-voltage” L-type channels. The data provided herein demonstrate that L-type channels in non-excitable cells such as T cells are voltage sensitive and have a molecular signature, that is, a characteristic I/V curve having small currents. Based on these data and the data provided supra showing that the channels expressed in non-excitable cells are the result of alternative splicing, producing an α-subunit having an exon 1 that has not been previously described, the L-type channels expressed in these cells have decreased voltage sensitivity compared to neuronal L-type channels.

### Example 13

### Functional Investigations Using siRNA

To determine the effect of inhibiting expression of Ca(v)1.3 polypeptides in a non-excitable cell type, Jurkat cells were stably transfected with various siRNA constructs targeting L-type channels. Specific siRNA sequences were generated as primers (Invitrogen) and cloned into the psiRNA-hHlzeo vector (Invitrogen) according to the manufacturer's instructions. The specific sequences included: RNAi-1 (LT1), AACTGTGAGCTGGACAAGAA (SEQ ID NO:109); RNAi-2 (LT2), AACAACAACTTCCAGACCTT (SEQ ID NO:110); RNAi-a (D1), AAGATGTTCAATGATGCCA (SEQ ID NO:111); RNAi-b (D2), AAGATGTTCAATGATGCCA (SEQ ID NO:112); and RNAi-2.2 (D3), AATAGGAACAATAACTTCC (SEQ ID NO:113). The RNAi-1 and RNAi-2 sequences generally target L-type channel sequences and the RNAi-a, RNAi-b, and RNAi-2.2 sequences specifically target Ca(v)1.3 sequences.

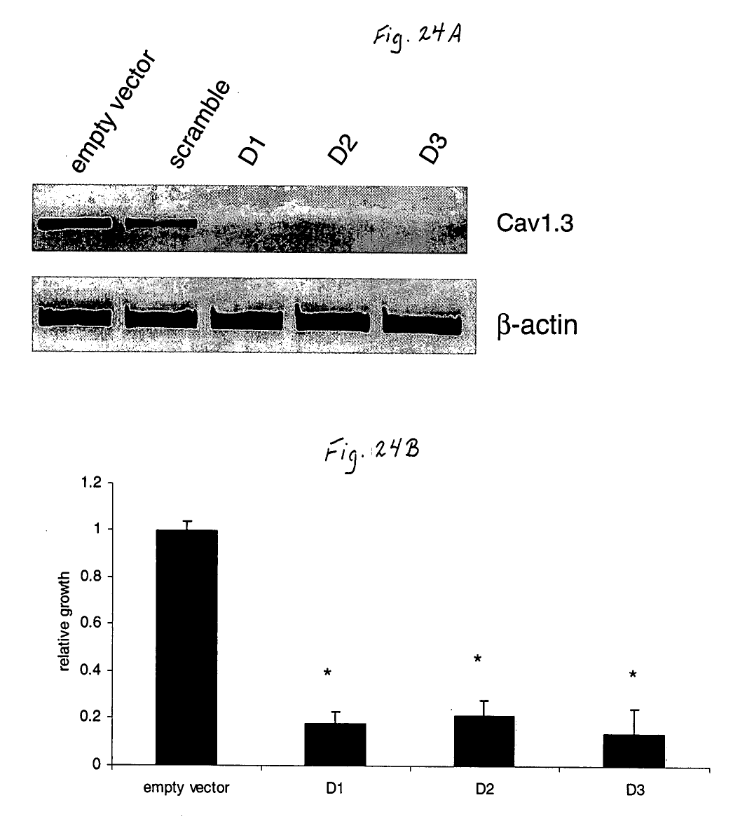

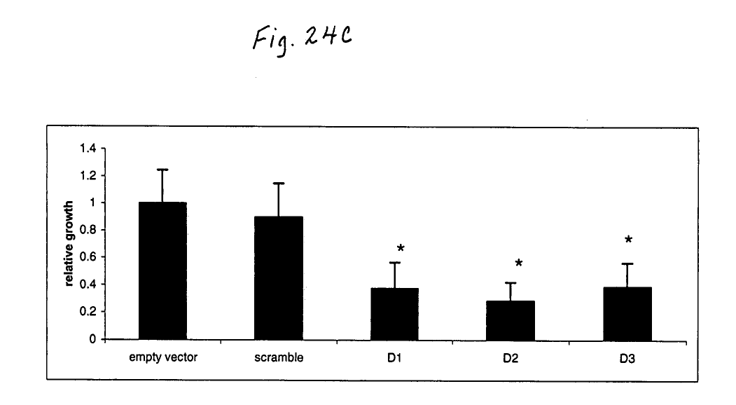

Jurkat cells were stably transfected with each of the siRNA constructs and pools of clones of each were analyzed. RT-PCR was performed on samples from the clones to detect expression of Ca(v)1.3. As shown in FIG. 24A, the Ca(v)1.3 siRNA-expressing clones had decreased Cav1.3 expression. β-Actin gene expression levels were approximately equal in each sample. These data show that siRNA expression of each of the constructs targeting Ca(v)1.3 resulted in a significant decrease in endogenous Ca(v)1.3 expression in Jurkat T cells, as compared to empty vector control and a scrambled siRNA-expressing sequence. In general, Ca(v)1.3 siRNA-expressing clones showed an average 50% decrease in overall cell numbers compared to controls.

An MTT assay (Molecular Probes) was used to further examine cell growth. The assay was performed according to the manufacturer's instructions. In these experiments, cells were transfected with empty vector, RNAi-1, or RNAi-2, cultured, and assayed for percent relative growth (using empty vector as a control). A decrease in relative cell growth was observed in Cav1.3-specific siRNA-expressing cells (FIG. 24B). The asterisks in FIG. 24B indicate p<0.05. In addition, a BrdU assay was performed on these cells. These experiments demonstrated decreased cell proliferation of Cav1.3-expressing siRNA clones compared to empty vector and scrambled clones (FIG. 24C). The asterisks indicate p<0.05. The decreased cell growth was not a result of apoptosis. Furthermore, cell cycle analysis with propidium iodide staining did not indicate a block at any given stage of the cell cycle, although a higher percentage of each of the Cav1.3-depleted cells were quiescent. Altogether, these data clearly demonstrate that Cav1.3 has a non-redundant function that is fundamental for T cell growth. Therefore, channels in the Ca(v)1.3 family (such as LVCa(v)1.3 channels) are involved in cellular proliferation and inhibition of such channels can be used to inhibit cell proliferation.

These data therefore demonstrate the compounds that inhibit L-type channels (e.g., LVCa(v)1.3 channels) are useful for inhibiting cellular proliferation and are candidate compounds for treating proliferative disorders involving cells containing such channels (e.g., non-excitable cells, including cells of the immune system).

### Example 14

### L-type Channels Are Distinct from Channels that Generate Icrac Currents in T Cells

Store-operated calcium flux has been described in non-excitable cells and IP3-mediated store-operated calcium entry is a well-recognized and characterized pathway of calcium influx. The channels involved in this mechanism are characterized by “Icrac” currents. To determine whether L-type channels are involved in the generation of Icrac currents in non-excitable cells, Icrac currents were assayed in Jurkat cells in which L-type channel expression was inhibited using siRNA.

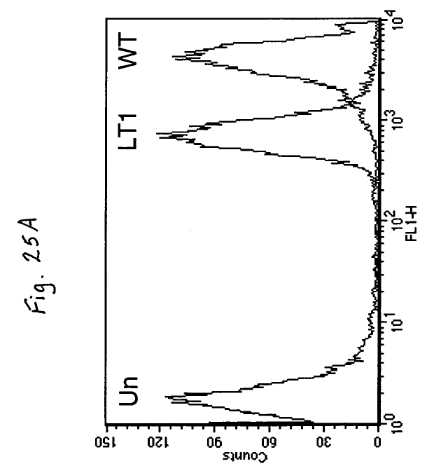

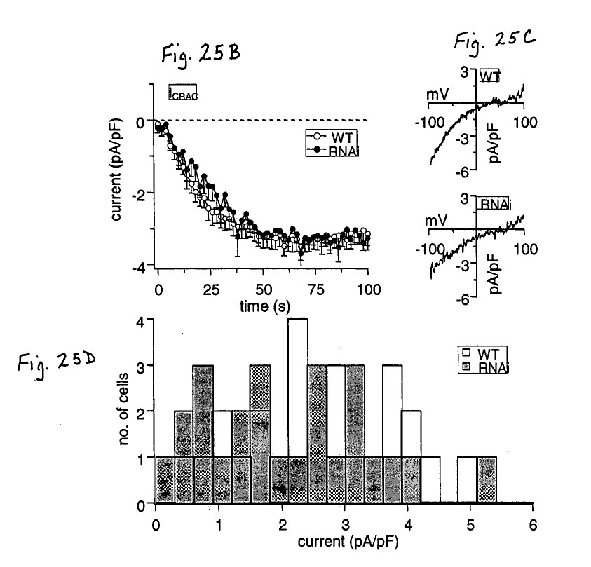

First, control wild type (WT) and L-type siRNA-expressing (LT1) Jurkat T cells were stained with DM-BODIPY and analyzed by flow cytometry (Un indicates unstained cells). The LT1 cells were transfected with an siRNA sequence that targets the majority of L-type channels, and was designed based on sequence consensus of the human L-type calcium channels. A representative trace of these cells is shown in FIG. 25A. As discussed above, these data show that stable expression of such siRNAs decreases the surface expression of L-type channels by about 90%. It was also noted that these clones exhibited a slow rate of growth, showing a similar phenotype to Cav1.3-depleted cells.

Whole cell patch clamp experiments were performed to examine Icrac currents. These experiments were generally performed as described for the experiments in Example 12 except that the intracellular pipette-filling solutions for I_{CRAC} measurements contained (in mM): Cs-glutamate 120, NaCl 8, MgCl₂ 3, InSP₃ 0.02, Cs-BAPTA 10, Hepes.CsOH 10, pH 7.2. To measure I_{CRAC}, voltage ramps of 50 ms duration spanning the voltage range of −100 to +100 mV were delivered from a holding potential of 0 mV at a rate of 0.5 Hz over a period of 200 to 400 seconds. For I_{CRAC} analysis, the very first ramps prior to current activation were digitally filtered at 2 kHz, pooled and used for leak-subtraction of all subsequent current records. The low-resolution temporal development of currents at a given potential was extracted from the leak-corrected individual ramp current records by measuring the current amplitudes at voltages of −80 mV or +80 mV, respectively.

FIG. 25B shows an average time course development of Icrac in WT (control) (n=7) and RNAi clone 25 (n=5) Jurkat cells. FIG. 25C shows two raw data traces of cells at 100 seconds of a whole cell experiment. The upper panel shows a wild type (Jurkat cell) and the lower panel shows a trace from an experiment using a cell transfected with an siRNA targeting L-type channels (RNAi clone 25). FIG. 25D is a current amplitude histogram of cells transfected with RNAi (clone 14) (n=20) and clone 25 (n=5) in comparison to current amplitude histogram of untransfected (WT) Jurkat cells (n=27). The average current size of RNAi cells was 2.1+−0.25 pA/pf (n=25), of WT average current size was 2.9+−0.36 pA/pF (n=27). No statistical difference was observed between the two data sets (Student's t-test). Thus, despite the phenotypic difference between wild type cells and cells having depleted Ca(v)1.3 channel expression, generation of Icrac currents in the L-type channel-diminished cells remained unaltered. Altogether, these data demonstrate that in a non-excitable cell in which expression of L-type channels is expressed, Icrac currents are still present and therefore L-type channels operate a calcium entry pathway that is distinct from that of Icrac in T cells.

Since siRNA targeting L-type channels inhibits expression of L-type channels, but Icrac currents are still present in cells expressing siRNA targeting L-type channels, L-type channels are distinct from the channels that operate Icrac in T cells. Thus, a novel, independent calcium entry pathway that is distinct from that of Icrac is herein demonstrated to exist in non-excitable (e.g., T cells). Thus, compounds that target the L-type channels and do not affect Icrac can be used to specifically target L-type channels that are expressed in non-excitable cells.

### Example 15

### Unique Structure of T cell Cav1.3 is Expressed in Human Blood Cells

To determine whether LVCa(v) is expressed in a non-immortal non-excitable cell type, RT-PCR targeting the first exon of LVCa(v)1.3 (i.e., exon A/B extending into exon 3) was performed. Briefly, the LVCa(v)1.3-specific sequence was cloned by PCR from human peripheral blood cell (PBC) cDNAs (BD Biosciences) with the following primers (5′-3′): CATCATGATGGAACCGCTGTT (SEQ ID NO:114) for the forward direction, and CATCTTCAGGGAATGGGATGTA (SEQ ID NO:115) for reverse. Thirty-five cycles were performed with an annealing temperature of 56° C. Sequence identities were confirmed by TA cloning (Invitrogen) and sequencing on an ABI instrument.

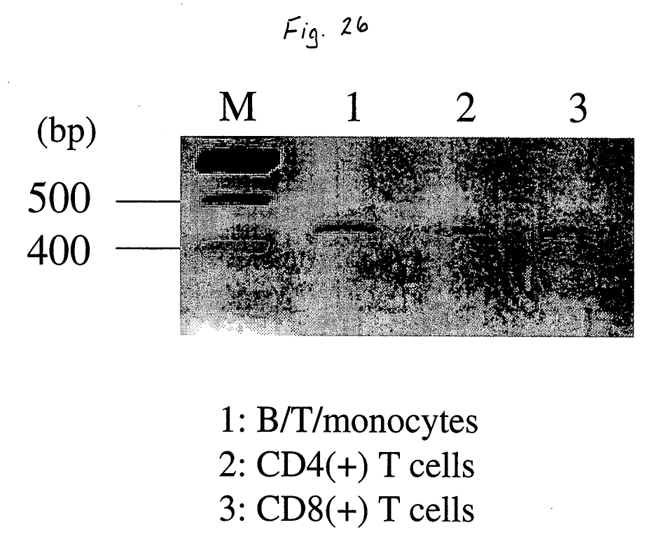

FIG. 26 shows the results of PCR amplification experiments on monocytes (lane 1), CD4(+) T cells, and CD8(+) T cells, shown with marker sequences (M). These results demonstrated the expression of LVCa(v)1.3 in all three cell types including native human blood cells. The presence of these sequences was confirmed by TA-cloning and sequencing.

These data demonstrate that the expression of the LVCa(v) variant is not limited to immortal cell types and is expressed in a normal non-excitable cell type.

### Example 16

### Over-Expression of T Cell Variant Cav1.3 Enhances T Cell Growth

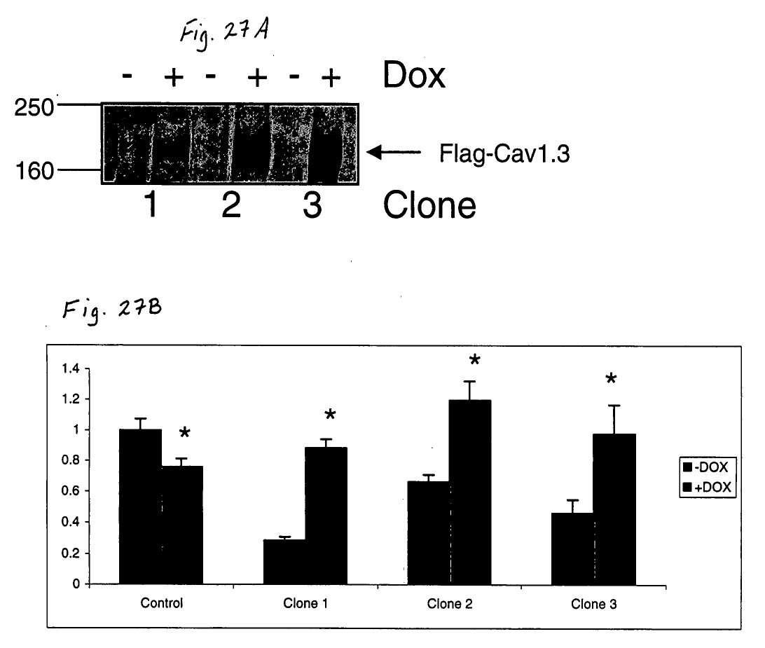

The unique structure of the LVCa(v)1.3 channel proteins and their prominence in non-excitable cell types (e.g., T cells) suggests they have a specific role in these cells. As discussed above, Ca(v)1.3 polypeptides appear to play a role in cellular proliferation. To further investigate this feature, Jurkat T-Rex cells (Invitrogen) were stably transfected with FLAG-tagged T cell variant Cav1.3 containing the A/B exon. Briefly, Cav1.3 cDNA that was obtained using RT-PCR of Jurkat cells was cloned into pcDNA™4/TO (Invitrogen) and stably transfected into the Jurkat T-Rex cell line. Expression of protein was determined by immunoprecipitation and immunoblotting with anti-FLAG M2 antibodies (Sigma-Aldrich, St. Louis, Mo.). Clones were selected based on their ability to express high levels of recombinant LVCav1.3 (containing exons A/B, no exon 33, and terminates after exon 44) and when induced with doxycycline (1 μg/ml for 72 hours). FIG. 27A shows that the cells expressed the recombinant Flag-LVCav1.3 (i.e., over-expressed) in three independent clones, producing a protein of the expected size (about 200 kDa).

A BrdU incorporation assay was used to assay cell growth in induced and uninduced cells. By 48-72 hours after induction, cells expressing LVCa(v)1.3 sequences had increased cell numbers compared to uninduced controls (about a 15% increase). FIG. 27B illustrates the increased cell growth at about 72 hours in the induced (i.e., LVCa(v)1.3-expressing) cells compared to the uninduced cells. LVCa(v)1.3 overexpression enhanced basal T cell growth and no mitogens were required to observe this effect. These data demonstrate that LVCa(v)1.3 has an effect on cell growth, e.g., cells over-expressing an LVCa(v) have enhanced cell growth.

These data complement the experiments demonstrating that inhibition of expression of Ca(v)1.3 inhibits cellular proliferation by demonstrating that over-expression of LVCa(v)1.3 increases cellular proliferation. These data also demonstrate the functionality of the LVCa(v)1.3 polypeptide. They also show that LVCav1.3 is part of a control pathway in T cells, distinct from store-operated calcium entry, that is specifically is required for cell growth. Thus, compounds that increase expression or activity of an LVCa(v)1.3 polypeptide in a cell (e.g., a non-excitable cell such as a T cell) are useful as compounds that can increase cellular proliferation. In addition, such overexpression can be used in models of disorders related to hyperproliferation of T cells or other cell types that express an LVCa(v)1.3 polypeptide. Expression of an LVCa(v)1.3 polypeptide in a cell from a subject suspected of having a proliferative disorder can be used to confirm the presence of the disorder and can serve as a guide for treatment (e.g., providing the subject with a compound that inhibits expression or activity of an LVCa(v)1.3 polypeptide.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated nucleic acid molecule encoding a polypeptide, wherein the polypeptide comprises an amino acid sequence selected from the group consisting of the amino acid sequence encoded by exon A, exon B, exon A/B, exon 11-13, exon 33A, exon 32-34, LV1Ca(v)1.3, LV2Ca(v)1.3, exon A′, exon B′, exon A′/B′, an LVCa(v)1.3 amino acid sequence terminating at the end of exon 44, an amino acid sequence terminating at the end of Exon 50, and variants thereof having one or more conservative amino acid substitutions.

2. The isolated nucleic acid molecule of claim 1, wherein the amino acid sequence is from a mammal.

3. The isolated nucleic acid molecule of claim 2, wherein the mammal is a human.

4. An isolated nucleic acid molecule, the nucleic acid molecule comprising a nucleic acid sequence selected from the group consisting of exon A, exon B, exon A/B, exon 11-13, exon 33A, exon 32-34, a LV1Ca(v)1.3 nucleic acid molecule, an LV2Ca(v)1.3 nucleic acid molecule, exon A′, exon B′, exon A′/B′, a nucleic acid molecule encoding an LVCa(v)1.3 amino acid sequence terminating at the end of exon 44, a nucleic acid molecule encoding an amino acid sequence terminating at the end of exon 50, and variants thereof having one or more substitutions resulting in conservative amino acid substitutions; or a complement thereof.

5. An isolated nucleic acid molecule comprising a polynucleotide sequence that hybridizes to a second polynucleotide sequence selected from the group consisting of exon A, exon B, exon A/B, exon 11-13, exon 33A, exon 32-34, LV1Ca(v)1.3, LV2Ca(v)1.3, exon A′, exon B′, exon A′/B′, a sequence encoding an LVCa(v)1.3 amino acid sequence terminating at the end of exon 44 or a portion thereof, a sequence encoding an amino acid sequence terminating at the end of exon 50, a 3′ untranslated sequence of an LVCa(v)1.3, a 5′ untranslated sequence of an LVCa(v)1.3, a 5′ untranslated sequence of an LVCa(1.1), and variants thereof having one or more substitutions resulting in conservative amino acid substitutions; or a complement thereof, the hybridization conditions comprising hybridization in 50% formamide at 42° C. and washing in 0.2×SSC and 0.1% SDS at 68° C.

6. A recombinant expression vector comprising the nucleic acid molecule of any one of claims 1-5.

7. A recombinant cultured cell comprising a nucleic acid molecule of any one of claims 1-5.

8. A recombinant cultured cell comprising a nucleic acid molecule of any one of claims 1-5, wherein expression of the nucleic acid molecule increases cell growth compared to a control cell.

9. A recombinant cultured cell comprising a polypeptide encoded by a nucleic acid sequence selected from the group consisting of exon A, exon B, exon A/B, exon 11-13, exon 33A, exon 32-34, an LV1Ca(v)1.3 nucleic acid molecule, an LV2Ca(v)1.3 nucleic acid molecule, exon A′, exon B′, exon A′/B′, a nucleic acid molecule encoding an LVCa(v)1.3 amino acid sequence terminating at the end of exon 44, a nucleic acid molecule encoding an LVCa(v) amino acid sequence terminating at the end of exon 50, and variants thereof having one or more conservative amino acid substitutions.

10. The recombinant cultured cell of claim 9, wherein the cell has increased cell growth compared to a control cell that does not comprise the polypeptide.

11. A recombinant cultured cell comprising a deletion in at least one allele of an LVCa(v) nucleic acid sequence, wherein the level of expression of the LVCa(v) polypeptide is reduced compared to a cell without the deletion, and wherein the polypeptide comprises an amino acid sequence selected from the group consisting of exon A, exon B, exon A/B, exon 11-13, exon 33A, exon 32-34, LV1Ca(v)1.3, LV2Ca(v)1.3, exon A′, exon B′, exon A′/B′, an LVCa(v)1.3 amino acid sequence terminating at the end of exon 44, an amino acid sequence terminating at the end of exon 50, and variants thereof having one or more conservative amino acid substitutions.

12. A recombinant cultured cell comprising a deletion in at least one allele of a LVCa(v) nucleic acid sequence, wherein the level of expression of the LVCa(v) nucleic acid sequence is reduced compared to a cell without the deletion, and wherein the gene comprises a nucleic acid sequence comprising a sequence selected from the group consisting of exon A, exon B, exon A/B, exon 11-13, exon 33A, exon 32-34, an LV1Ca(v)1.3 nucleic acid molecule, an LV2Ca(v)1.3 nucleic acid molecule, exon A′, exon B′, exon A′/B′, a sequence encoding an LVCa(v)1.3 amino acid sequence terminating at the end of exon 44 or a portion thereof, a sequence encoding an amino acid sequence terminating at the end of exon 50, a 3′ untranslated sequence of an LVCa(v)1.3, a 5′ untranslated sequence of an LVCa(v)1.3, a 5′ untranslated sequence of an LVCa(1.1), and variants thereof having one or more substitutions resulting in conservative amino acid substitutions; or a complement thereof.

13. The recombinant cultured cell of claim 12, wherein the cell is a DT40 cell or a Jurkat cell.

14. A substantially pure polypeptide comprising an amino acid sequence selected from the group consisting of exon A, exon B, an exon A/B, exon 11-13, exon 33A, exon 32-34, LV1Ca(v)1.3, LV2Ca(v)1.3, exon A′, exon B′, exon A′/B′, an LVCa(v)1.3 amino acid sequence terminating at the end of exon 44, an amino acid sequence terminating at the end of exon 50, and variants thereof having one or more conservative amino acid substitutions.

15. The polypeptide of claim 14, consisting of the amino acid sequence of exon A, exon B, exon A/B, exon 11-13, exon 33A, exon 32-34, LV1Ca(v)1.3, LV2Ca(v)1.3, exon A′, exon B′, exon A′/B′, an LVCa(v)1.3 amino acid sequence terminating at the end of exon 44 (SEQ ID NO. 2), an amino acid sequence terminating at the end of exon 50, and variants thereof having one of more conservative amino acid substitutions.

16. The polypeptide of claim 14 or 15, wherein the polypeptide can exhibit LVCa(v) activity.

17. The polypeptide of claim 14 or 15, wherein recombinant expression of the polypeptide in a cell modulates cell growth compared to a control.

18. A substantially pure polypeptide encoded by a nucleic acid sequence that hybridizes to any of exon A, exon B, exon A/B, exon 11-13, exon 33A, exon 32-34, LV1Ca(v)1.3, LV2Ca(v)1.3, exon A′, exon B′, exon A′/B′, a sequence encoding an LVCa(v)1.3 amino acid sequence terminating at the end of Exon 44 or a portion thereof, a sequence encoding an amino acid sequence terminating at the end of Exon 50, a 3′ untranslated sequence of an LVCa(v)1.3, a 5′ untranslated sequence of an LVCa(v)1.3, a 5′ untranslated sequence of an LVCa(1.1), and variants thereof having one or more substitutions resulting in conservative amino acid substitutions, or a complement thereof, the hybridization conditions comprising hybridization in 50% formamide at 42° C. and washing in 0.2×SSC and 0.1% SDS at 68° C.

19. A method for identifying an agent that modulates expression of an LVCa(v) gene in a cell, the method comprising:
(a) obtaining a test cell that expresses an LVCa(v) polypeptide;
(b) contacting the test cell with a test agent;
(c) measuring the level of expression of the LVCa(v) mRNA in the test sample exposed to the test agent;
(d) determining that the test agent is a modulator of LVCa(v) expression if the level of expression of the LVCa(v) mRNA in the test sample exposed to the test agent is less than the level of expression of the LVCa(v) in a test cell that was not contacted with the test agent.

20. The method of claim 19, wherein the cell is a non-excitable cell.

21. The method of claim 19, wherein the LVCa(v) is an LVCa(v)1.3

22. The method of claim 19, wherein step (c) comprises contacting the test sample with a nucleic acid molecule that hybridizes to the LVCa(v) mRNA under stringent conditions.

23. The method of claim 19, wherein the test agent is an antisense agent or an RNAi agent.

24. A method for identifying an agent that modulates expression of an LVCa(v) polypeptide in a cell, the method comprising:
(a) obtaining a test cell that expresses an LVCa(v) polypeptide;
(b) contacting the test cell with a test agent;
(c) measuring the level of expression of the LVCa(v) polypeptide in the test cell contacted with the test agent;
(d) determining that the test agent is an agent that modulates expression of the LVCa(v) polypeptide if the level of expression of the LVCa(v) polypeptide in the test sample contacted with the test agent is less than the level of expression in a test cell that was not contacted with the test agent.

25. The method of claim 24, wherein the cell is a non-excitable cell.

26. The method of claim 24, wherein step (c) comprises contacting the test sample with an agent that binds to the LVCa(v) polypeptide.

27. The method of claim 24, wherein the test agent is an antibody.

28. The method of claim 27, wherein the antibody is a monoclonal antibody.

29. The method of claim 27, wherein the test agent is a single chain antibody, a Fab, or an epitope-binding fragment of an antibody.

30. The method of claim 24, wherein the test agent is detectably labeled.

31. The method of claim 30, wherein the detectable label is a radioactive label, a fluorescent label, a chemiluminescent label, or a bioluminescent label.

32. A method for identifying an agent that modulates activity of an LVCa(v) polypeptide in a cell, the method comprising
(a) obtaining a test sample comprising a cell that expresses an LVCa(v) polypeptide;
(b) contacting the test sample with a test agent;
(c) measuring the level of activity of the LVCa(v) polypeptide in the test sample contacted with the test agent;
(d) determining that the test agent is an agent that modulates an LVCa(v) activity if the level of activity of the LVCa(v) polypeptide in the test sample contacted with the test agent is less than the level of expression in test sample that was not contacted with the test agent.

33. The method of claim 32, wherein the test agent is a dihydropyridine, phenylalkylamine, benzodiazepine, benzothiazapine, diarylaminopropylamine ether, or benzimidazole-substituted tetralin.

34. The method of claim 32, wherein the test agent can inhibit the activity of the LVCa(v) polypeptide in vitro by at least about 50% at a concentration of less than about 1 μm.

35. The method of claim 32, wherein the activity of the LVCa(v) polypeptide is modulation of cell growth.

36. The method of claim 32, wherein the activity of the LVCa(v) polypeptide is modulation of calcium flux.

37. The method of claim 32, wherein the test agent inhibits phosphorylation of the LVCa(v).

38. The method of claim 32, wherein the LVCa(v) is an LVCa(v)1.3 polypeptide.

39. A method of inhibiting calcium influx in a non-excitable cell, the method comprising inhibiting the activity of an LVCa(v) polypeptide that is expressed in the non-excitable cell.

40. The method of claim 39, wherein the activity of the LVCa(v) polypeptide is increased cell growth.

41. The method of claim 39, wherein the LVCa(v) polypeptide is an LVCa(v)1.3 polypeptide.

42. A method of inhibiting calcineurin activity in a non-excitable cell, the method comprising inhibiting the activity of an LVCa(v) polypeptide that is expressed in the non-excitable cell.

43. A method of inhibiting NFAT activity in a non-excitable cell, the method comprising inhibiting activity of an LVCa(v) polypeptide that is expressed in the non-excitable cell.

44. A method of inhibiting IL-2 production in a non-excitable cell, the method comprising inhibiting the activity of an LVCa(v) polypeptide that is in the non-excitable cell.

45. A method of inhibiting secretion of a cytokine in a non-excitable cell selected from the group consisting of a lymphocyte, a mast cell, an HEK293 cell, and a Jurkat cell, the method comprising inhibiting the expression or activity of an LVCa(v) polypeptide in the non-excitable cell.

46. A method of inhibiting the activity of a Ca²⁺-activated gene product in a non-excitable cell, the method comprising the step of inhibiting the activity of an LVCa(v) polypeptide in the non-excitable cell.

47. A method of inhibiting proliferation of a non-excitable cell, the method comprising selectively inhibiting the activity of an LVCa(v) polypeptide in the non-excitable cell.

48. The method of claim 47, wherein phosphorylation of the LVCa(v) polypeptide is inhibited.

49. The method of claim 47, wherein the non-excitable cell is a cancer cell.

50. A method of inhibiting differentiation of a non-excitable cell, the method comprising the step of inhibiting the activity of an LVCa(v) polypeptide in the non-excitable cell.

51. The method of claim 50, wherein phosphorylation of the LVCa(v) polypeptide is inhibited.

52. A method of inhibiting immune cell function, the method comprising inhibiting the activity of an LVCa(v) polypeptide in the cell.

53. The method of any one of claims 39-52, wherein the activity of the LVCa(v) polypeptide is inhibited in vitro by at least about 50% using an agent that is present at a concentration of less than about 1 μM.

54. The method of claim any one of claims 39-52, wherein the agent is a dihydropyridine, phenylalkylamine, benzodiazepine, benzothiazapine, diarylaminopropylamine ether, or benzimidazole-substituted tetralin.

55. The method of claim 54, wherein the agent is a dihydropyridine.

56. The method of any one of claims 39-52, wherein the LVCa(v) polypeptide is an LVCa(v)1.3 polypeptide.

57. The method of any one of claims 39-52, wherein the LVCa(v) polypeptide is an LVCa(v)1.3 polypeptide and comprises an amino acid sequence selected from the group depicted in FIGS. 1B, 1D, 2B, 2D, 2F, 3B, 4B, 6B, 8B, an amino acid sequence terminating at the end of a Ca(v)1.3 exon 44, an amino acid sequence terminating at the end of a Ca(v)1.3 exon 50 and variants thereof having one or more conservative amino acid substitutions.

58. The method according to any one of claims 39-52, wherein the non-excitable cell is selected from the group consisting of a lymphocyte, mast cell, and a cell derived from a lymphocyte or mast cell.

59. The method of claim 58, wherein the non-excitable cell is a T cell, a B cell, or a DT40 chicken cell.

60. The method of claim 58, wherein the non-excitable cell is a Jurkat cell.

61. A method for treating or preventing a cancer, an immune system disorder, or an inflammatory condition in a subject, the method comprising inhibiting expression or activity of an LVCa(v) polypeptide that is expressed in a non-excitable cell.

62. The method of claim 61, wherein the immune system disorder is an allergic disorder, an immune system-related cancer, or an autoimmune disorder.

63. The method of claim 62, wherein the disorder is an autoimmune disorder that is selected from the group consisting of multiple sclerosis, myasthenia gravis, autoimmune neuropathies, Guillain-Barre, autoimmune uveitis, autoimmune hemolytic anemia, pernicious anemia, autoimmune thrombocytopenia, temporal arteritis, anti-phospholipid syndrome, vasculitides, Wegener's granulomatosis, Behcet's disease, psoriasis, dermatitis herpetiformis, pemphigus vulgaris, vitiligo, Crohn's disease, ulcerative colitis, primary biliary cirrhosis, and autoimmune hepatitis, Type 1 or immune-mediated diabetes mellitus, Grave's disease, Hashimoto's thyroiditis, autoimmune oophoritis and orchitis, autoimmune disease of the adrenal gland; rheumatoid arthritis, systemic lupus erythematosus, scleroderma, polymyositis, dermatomyositis, ankylosing spondylitis, Sjogren's syndrome and graft-versus-host disease.

64. The method of claim 62, wherein the disorder is an immune system-related cancer that is selected from the group consisting of Kaposi's sarcoma and leukemia.

65. The method of claim 61, wherein the activity of the LVCa(v) polypeptide is inhibited and inhibition in vitro is at least about 50% using an agent that is present at a concentration of less than about 1 μM.

66. The method of claim 65, wherein the agent is a dihydropyridine, phenylalkylamine, benzodiazepine, benzothiazapine, diarylaminopropylamine ether, or benzimidazole-substituted tetralin.

67. The method of claim 65, wherein the agent is a dihydropyridine.

68. The method of claim 61, wherein the LVCa(v) polypeptide is an LVCa(v)1.3 polypeptide.

69. The method of clam 61, wherein the LVCa(v) polypeptide is an LVCa(v)1.3 polypeptide that comprises an amino acid sequence selected from the group consisting of the sequence depicted in any one of FIGS. 1B, 1D, 2B, 2D, 2F, 3B, 4B, 6B, 8B, an amino acid sequence terminating at the end of an exon 44, an amino acid sequence terminating at the end of an exon 50 and variants thereof having one or more conservative amino acid substitutions.

70. The method of claim 61, wherein the non-excitable cell is selected from the group consisting of a tumor cell, lymphocyte, mast cell, and a cell derived from a lymphocyte or mast cell.

71. A cell line derived from a non-excitable cell that can overexpress an LVCa(v) polypeptide.

72. The cell line of claim 71, wherein the LVCa(v) polypeptide is an LVCa(v)1.3 polypeptide.

73. The cell line of claim 72, wherein the LVCa(v)1.3 polypeptide comprises an amino acid sequence selected from the group consisting of amino acid sequence selected from the group depicted in FIGS. 1B, 1D, 2B, 2D, 2F, 3B, 4B, 6B, 8B, an amino acid sequence terminating at the end of exon 44 (SEQ ID NO. 2), an amino acid sequence terminating at the end of exon 50 (SEQ ID NO. 3) and variants thereof having one or more conservative amino acid substitutions.

74. An isolated LVCa(v) polypeptide produced by cell of any one of claims 71-73.

75. A method for identifying an LVCa(v) polypeptide that is differentially expressed in two or more non-excitable cell types, comprising quantitatively measuring the amount of mRNA encoding different LVCa(v) polypeptides in each cell type.

76. The method of claim 75, further comprising determining the expression profile of the LVCa(v) polypeptides in each cell type.

77. A method for identifying a candidate modulator of an LVCa(v) polypeptide in a non-excitable cell, the method comprising
(a) providing a non-excitable cell that can express one or more LVCa(v) polypeptides;
(b) contacting the cell with a test agent;
(c) measuring the ability of the test agent to inhibit calcium influx modulated by one or more LVCa(v) polypeptides that are differentially expressed in the cell, wherein a test agent that inhibits calcium influx modulated by one or more LVCa(v) polypeptides in the cell is a candidate modulator of an LVCa(v) polypeptide.

78. The method of claim 77, wherein the differential expression occurs between two or more different tissue types.

79. The method of claim 78, wherein the tissue types are thymus tissue and spleen tissue.

80. The method of claim 77, wherein the differential expression occurs between two or more different cell types.

81. The method of claim 80, wherein the cell types are T cells, mast cells, and B cells.

82. The method of claim 77, wherein the ability of the test agent to inhibit calcium influx is measured by assaying for one or more of the following activities:
calcineurin activity, NFAT activity, or IL-2 activity.

83. The method of claim 77, wherein expression of the LVCa(v) polypeptide is decreased when the cell is activated.

84. The method of claim 77, wherein expression of the LVCa(v) polypeptide is increased when the cell is deactivated.

85. The method of claim 77, wherein the expression of the LVCa(v) polypeptide is modulated when the cell is undergoing differentiation.

86. The method of claim 77, wherein the differentially expressed LVCa(v) polypeptides are identified using quantitative PCR.

87. A method of screening for a modulator of an LVCa(v) polypeptide in a cell, the method comprising
(a) providing a non-excitable cell that can express one or more LVCa(v) polypeptides;
(b) contacting the cell with a test agent; and
(c) evaluating the ability of the test agent to inhibit calcium influx modulated by one or more of the LVCa(v) polypeptides, wherein inhibition of calcium influx in the presence of the test agent compared to a reference that was not contacted with the test agent indicates that the test agent is a modulator of an LVCa(v) polypeptide.

88. The method of claim 87, wherein the ability of the test agent to inhibit calcium influx is measured in a cell line that overexpresses an LVCa(v)1.3 polypeptide.

89. The method of claim 87, wherein the cell expresses at least two different LVCa(v) polypeptides that are differentially expressed between two or more different tissue types.

90. The method of claim 89, wherein the tissue types are thymus and spleen.

91. The method of claim 87, wherein at least two LVCa(v) polypeptides are expressed and the LVCa(v) polypeptides are differentially expressed between two or more different cell types.

92. The method of claim 91, wherein one or more of the LVCa(v) polypeptides are differentially expressed between a tumor cell and a normal cell.

93. The method of claim 91, wherein the cell types are T cells, mast cells, or B cells.

94. The method of claim 87, wherein the LVCa(v) polypeptide is differentially expressed when the cell is activated compared to a cell that is not activated.

95. A method for identifying a modified agent that can modulate the activity of an LVCa(v) polypeptide in a cell, the method comprising
(a) providing an agent that modulates the activity of an LVCa(v) polypeptide in a cell;
(b) modifying the agent by producing a chemical analog or derivative thereof, thereby producing a modified agent; and
(c) measuring the ability of the modified agent to modulate the activity of an LVCa(v) polypeptide in a non-excitable cell, wherein increased modulation in the presence of the modified agent compared to the agent indicates that the modified agent is an improved agent.

96. The method of claim 95, wherein the modified agent modulates the LVCa(v) polypeptide in a non-excitable cell at or below a chosen threshold level.

97. The method of claim 96, wherein the threshold level is 50% inhibition of the LVCa(v) polypeptide in vitro at about 100 nM.

98. The method of claim 96, wherein the threshold level is 50% inhibition of the LVCa(v) polypeptide in vitro at about 100 nM.

99. The method of any one of claims 95-98, wherein the agent is a dihydropyridine, phenylalkylamine, benzodiazepine, benzothiazapine, diarylaminopropylamine ether, benzimidazole-substituted tetralin, or a derivative thereof.

100. The method of any one of claims 95-98, wherein the agent is a dihydropyridine.

101. The method according to claim 95, wherein the ability of the modified agent to modulate the activity of the LVCa(v) polypeptide in the non-excitable cell is measured by evaluating bulk calcium influx.

102. A modified agent identified according to the method of claim 95.

103. The method of claim 95, wherein the non-excitable cell is a T cell.

104. A method for identifying a candidate modulator of activity of an LVCa(v) polypeptide in a non-excitable cell, the method comprising:
(a) providing a non-excitable cell;
(b) contacting the cell with a test compound;
(c) measuring the ability of the test compound to inhibit calcineurin activity in the non-excitable cell; and
(d) testing the ability of the compound to inhibit bulk calcium influx in the non-excitable cell, wherein, a compound that can inhibit calcineurin activity and bulk calcium influx in the non-excitable cell is a candidate modulator of the LVCa(v) polypeptide.

105. A modulator of an LVCa(v) polypeptide in a non-excitable cell identified by the method of claim 104.

106. A method for identifying a nucleic acid sequence that can inhibit expression of an LVCa(v) gene, the method comprising
(a) transfecting a cell with an expression vector comprising a nucleotide sequence comprising at least 19 contiguous nucleotides of an LVCa(v) cDNA sequence;
(b) culturing the cell under conditions sufficient for expression of the nucleotide sequence,
(c) measuring the level of expression of the LVCa(v) mRNA or polypeptide in the cell, wherein a decrease in the level of expression of the LVCa(v) mRNA or polypeptide indicates that the nucleic acid sequence can inhibit expression of the LVCa(v).

107. The method of claim 106, wherein the cDNA is selected from the group consisting of a sequence depicted in any one of FIGS. 1A, 1C, 2A, 2C, 2E, 3A, 4A, 6A, 7, 8A, 9, 10A, 10C, 5C, 5D, 10E, degenerate variants thereof, or a complement thereof.

108. The method of claim 106, wherein calcium influx is assayed in the cell and wherein calcium influx is inhibited when the nucleic acid sequence is expressed.

109. An RNAi agent derived from a nucleic acid sequence selected from the group consisting of a sequence depicted in any one of FIGS. 1A, 1C, 2A, 2C, 2E, 3A, 4A, 6A, 7, 8A, 9, 10A, 10C, 5C, 5D, 10E, degenerate variants thereof, or a complement thereof.

110. A method for inhibiting expression of a LVCa(v) nucleic acid sequence, the method comprising introducing an RNAi agent complementary to at least 19 contiguous nucleotides of the LVCa(v) nucleic acid sequence into a cell.

111. A method for inhibiting expression of an LVCa(v) gene in a subject in need thereof, the method comprising administering a therapeutically effective amount of an RNAi agent targeted to an LVCa(v) nucleotide sequence to the subject.

112. The method of claim 110 or 111, wherein the RNAi agent is an RNAi agent of claim 109.

113. An antisense agent derived from a nucleic acid sequence selected from the group consisting of a sequence depicted in any one of FIGS. 1A, 1C, 2A, 2C, 2E, 3A, 4A, 6A, 7, 8A, 9, 10A, 10C, 5C, 5D, 10E, degenerate variants thereof, or a complement thereof.

114. A method for inhibiting expression of an LVCa(v) gene in a cell, the method comprising introducing an antisense agent complementary to a portion of the nucleotide sequence of the LVCa(v) gene into the cell.

115. A method for inhibiting expression of an LVCa(v) gene in a subject in need thereof, the method comprising administering therapeutically effective amount of an antisense agent complementary to a portion of the LVCa(v) gene to the subject.

116. The method of claim 114 or 115, wherein the antisense agent is the antisense agent of claim 113.

117. A calcium channel comprising an LVCa(v) polypeptide or variant thereof comprising one of more conservative substitutions, and when the LVCa(v) polypeptide or variant is expressed in a cell, the cell exhibits an L-type current having a reversal potential of about 0 mV and a peak amplitude of about 3-5 pA.

118. The calcium channel of claim 117, wherein the I/V curve of the cell has the characteristics of FIG. 23.

119. The calcium channel of claim 117, wherein the cell is a non-excitable cell and the LVCa(v) polypeptide is a recombinant LVCa(v) polypeptide.

120. A calcium channel comprising an LVCa(v) polypeptide, wherein activity of the calcium channel is modulated by phosphorylation of the LVCa(v) polypeptide.

121. The calcium channel of claim 120, wherein the LVCa(v) polypeptide is an LVCa(v)1.3 polypeptide and activity is modulated by phosphorylation of the A/V exon of the LVCa(v)1.3 polypeptide.

122. A calcium channel that is expressed in a T cell, the calcium channel comprising a polypeptide, wherein activity of the channel is modulated by phosphorylation of an N-terminus sequence of the polypeptide.

123. The calcium channel of claim 122, wherein the N-terminus sequence is encoded by the first two exons of the mRNA encoding the polypeptide.

124. The calcium channel of claim 122, wherein the calcium channel comprises an LVCa(v) polypeptide or variant thereof comprising one or more conservative amino acid substitutions, and the channel is modulated by phosphorylation of the A/B exon.

125. The calcium channel of claim 124, wherein the A/B exon is phosphorylated at the TSS site.

126. A method of modulating calcium influx in a cell, the method comprising contacting a cell with a compound that affects phosphorylation of an LVCa(v)1.3 polypeptide.

127. The method of claim 126, wherein the cell is a non-excitable cell.

128. The method of claim 126, wherein the cell is a T cell.

129. The method of claim 126, wherein the compound affects phosphorylation of exon A/B of an LVCa(v)1.3 polypeptide.

130. A method of modulating cell proliferation, the method comprising contacting a cell with a compound that affects phosphorylation of an LVCa(v)1.3 polypeptide.

131. The method of claim 130, wherein the compound affects phosphorylation of exon A/B of an LVCa(v)1.3 polypeptide.

132. A method of inhibiting calcium influx into a non-excitable cell that expresses an LVCa(v) polypeptide, the method comprising contacting the cell with a selective inhibitor of the LVCa(v) polypeptide.

133. The method of claim 132, wherein the LVCa(v) polypeptide is an LVCa(v)1.3 polypeptide.

134. A method of identifying a subject having a proliferative cell disorder or who is at risk of developing a proliferative cell disorder, the method comprising
(a) obtaining a sample from the subject; and
(b) determining whether the subject has an aberrant level of expression of an LVCa(1.3), wherein an aberrant level of expression of an LVCa(1.3) compared to the level of expression in a normal population indicates that the subject has a proliferative cell disorder or is at risk for developing a proliferative cell disorder.

135. The method of claim 134, wherein the level of expression of the LVCa(1.3) is elevated compared to a normal population.

136. The method of claim 134, wherein the proliferative cell disorder includes undesirable proliferation of T cells.

137. The method of claim 134, wherein the level of expression of the LVCa(1.3) is decreased compared to a normal population.

138. The method of claim 134, wherein the proliferative cell disorder includes a low level of T cell proliferation compared to a normal population.


SEQUENCE LISTING (126 sequences)

SEQ ID NO: 1 - DNA, 12 bp, Homo sapiens
atgttttacatc

SEQ ID NO: 2 - protein, 4 aa, Homo sapiens
MFYI

SEQ ID NO: 3 - DNA, 63 bp, Homo sapiens
atgatggaaccgctgtttagatgcaggaaaacaagctccaggctcccactgattctacatgat

SEQ ID NO: 4 - protein, 21 aa, Homo sapiens
MMEPLFRCRKTSSRLPLILHD

SEQ ID NO: 5 - DNA, 75 bp, Homo sapiens
atgttttacatcatgatggaaccgctgtttagatgcaggaaaacaagctccaggctcccactgattctacatgat

SEQ ID NO: 6 - protein, 25 aa, Homo sapiens
MFYIMMEPLFRCRKTSSRLPLILHD

SEQ ID NO: 7 - protein, 21 aa, Homo sapiens
MMEPLFRCRKTSSRLPLILHD

SEQ ID NO: 8 - protein, 25 aa, Homo sapiens
MFYIMMEPLFRCRKTSSRLPLILHD

SEQ ID NO: 9 - DNA, 84 bp, Homo sapiens
cactatttcactgatgcatggaacacttttgatgccttaattgttgttggtagcgtcgttgatattgctataactgaagtgaat

SEQ ID NO: 10 - protein, 28 aa, Homo sapiens
HYFTDAWNTFDALIVVGSVVDIAITEVN

SEQ ID NO: 11 - DNA, 213 bp, Homo sapiens
gggtattttagtgacgcctggaacacgtttgactccctcatcgtaatcggcagcattatagacgtggccctcagcgaagcagacaactctgaagagagcaatagaatctccatcacctttttccgtcttttccgagtgatgcgattggtgaagcttctcagcaggggggaaggcatccggacattgctgtggacttttattaagtcctttcag

SEQ ID NO: 12 - protein, 71 aa, Homo sapiens
GYFSDAWNTFDSLIVIGSIIDVALSEADNSEESNRISITFFRLFRVMRLVKLLSRGEGIRTLLWTFIKSFQ

SEQ ID NO: 13 - DNA, 115 bp, Homo sapiens
ctagcatgcccaccagcgagactgagtctgtgaacacagagaacgtcagcggtgaaggcgagaaccgaggctgctgtggaagtctctgtcaagccatctcaaaatccaaactcag

SEQ ID NO: 14 - protein, 37 aa, Homo sapiens
SMPTSETESVNTENVSGEGENRGCCGSLCQAISKSKL

SEQ ID NO: 15 - DNA, 368 bp, Homo sapiens
agaaatggtgccctgcttggaaaccatgtcaatcatgttaatagtgataggagagattcccttcagcagaccaataccacccaccgtcccctgcatgtccaaaggccttcaattccacctgcaagtgatactgagaaaccgctgtttcctccagcaggaaattcggtgtgtcataaccatcataaccataattccataggaaagcaagttcccacctcaacaaatgccaatctcaataatgccaatatgtccaaagctgcccatggaaagcggcccagcattgggaaccttgagcatgtgtctgaaaatgggcatcattcttcccacaagcatgaccgggagcctcagagaaggtccagtgtgaaaag

SEQ ID NO: 16 - protein, 123 aa, Homo sapiens
RNGALLGNHVNHVNSDRRDSLQQTNTTHRPLHVQRPSIPPASDTEKPLFPPAGNSVCHNHHNHNSIGKQVPTSTNANLNNANMSKAAHGKRPSIGNLEHVSENGHHSSHKHDREPQRRSSVKR

SEQ ID NO: 17 - DNA, 5376 bp, Homo sapiens
atgttttacatcatgatggaaccgctgtttaaatgcaggaaaacaagctccaggctcccactgattctacatgatgaggcaaactatgcaagaggcaccagacttcctctttctggtgaaggaccaacttctcagccgaatagctccaagcaaactgtcctgtcttggcaagctgcaatcgatgctgctagacaggccaaggctgcccaaactatgagcacctctgcacccccacctgtaggatctctctcccaaagaaaacgtcagcaatacgccaagagcaaaaaacagggtaactcgtccaacagccgacctgcccgcgcccttttctgtttatcactcaataaccccatccgaagagcctgcattagtatagtggaatggaaaccatttgacatatttatattattggctatttttgccaattgtgtggccttagctatttacatcccattccctgaagatgattctaattcaacaaatcataacttggaaaaagtagaatatgccttcctgattatttttacagtcgagacatttttgaagattatagcgtatggattattgctacatcctaatgcttatgttaggaatggatggaatttactggattttgttatagtaatagtaggattgtttagtgtaattttggaacaattaaccaaagaaacagaaggcgggaaccactcaagcggcaaatctggaggctttgatgtcaaagccctccgtgcctttcgagtgttgcgaccacttcgactagtgtcaggggtgcccagtttacaagttgtcctgaactccattataaaagccatggttcccctccttcacatagcccttttggtattatttgtaatcataatctatgctattataggattggaactttttattggaaaaatgcacaaaacatgtttttttgctgactcagatatcgtagctgaagaggacccagctccatgtgcgttctcagggaatggacgccagtgtactgccaatggcacggaatgtaggagtggctgggttggcccgaacggaggcatcaccaactttgataactttgcctttgccatgcttactgtgtttcagtgcatcaccatggagggctggacagacgtgctctactgggtaaatgatgcgataggatgggaatggccatgggtgtattttgttagtctgatcatccttggctcatttttcgtccttaacctggttcttggtgtccttagtggagaattctcaaaggaaagagagaaggcaaaagcacggggagatttccagaagctccgggagaagcagcagctggaggaggatctaaagggctacttggattggatcacccaagctgaggacatcgatccggagaatgaggaagaaggaggagaggaaggcaaacgaaatactagcatgcccaccagcgagactgagtctgtgaacacagagaacgtcagcggtgaaggcgagaaccgaggctgctgtggaagtctctgtcaagccatctcaaaatccaaactcagccgacgctggcgtcgctggaaccgattcaatcgcagaagatgtagggccgccgtgaagtctgtcacgttttactggctggttatcgtcctggtgtttctgaacaccttaaccatttcctctgagcactacaatcagccagattggttgacacagattcaagatattgccaacaaagtcctcttggctctgttcacctgcgagatgctggtaaaaatgtacagcttgggcctccaagcatatttcgtctctcttttcaaccggtttgattgcttcgtggtgtgtggtggaatcactgagacgatcttggtggaactggaaatcatgtctcccctggggatctctgtgtttcggtgtgtgcgcctcttaagaatcttcaaagtgaccaggcactggacttccctgagcaacttagtggcatccttattaaactccatgaagtccatcgcctcgctgttgcttctgctttttctcttcattatcatcttttccttgcttgggatgcagctgtttggcggcaagtttaattttgatgaaacgcaaaccaagcggagcacctttgacaatttccctcaagcacttctcacagtgttccagatcctgacaggcgaagactggaatgctgtgatgtacgatggcatcatggcttacgggggcccatcctcttcaggaatgatcgtctgcatctacttcatcatcctcttcatttgtggtaactatattctactgaatgtcttcttggccatcgctgtagacaatttggctgatgctgaaagtctgaacactgctcagaaagaagaagcggaagaaaaggagaggaaaaagattgccagaaaagagagcctagaaaataaaaagaacaacaaaccagaagtcaaccagatagccaacagtgacaacaaggttacaattgatgactatagagaagaggatgaagacaaggacccctatccgccttgcgatgtgccagtaggggaagaggaagaggaagaggaggaggatgaacctgaggttcctgccggaccccgtcctcgaaggatctcggagttgaacatgaaggaaaaaattgcccccatccctgaagggagcgctttcttcattcttagcaagaccaacccgatccgcgtaggctgccacaagctcatcaaccaccacatcttcaccaacctcatccttgtcttcatcatgctgagcagcgctgccctggccgcagaggaccccatccgcagccactccttccggaacacgatactgggttactttgactatgccttcacagccatctttactgttgagatcctgttgaagatgacaacttttggagctttcctccacaaaggggccttctgcaggaactacttcaatttgctggatatgctggtggttggggtgtctctggtgtcatttgggattcaatccagtgccatctccgttgtgaagattctgagggtcttaagggtcctgcgtcccctcagggccatcaacagagcaaaaggacttaagcacgtggtccagtgcgtcttcgtggccatccggaccatcggcaacatcatgatcgtcaccaccctcctgcagttcatgtttgcctgtatcggggtccagttgttcaaggggaagttctatcgctgtacggatgaagccaaaagtaaccctgaagaatgcaggggacttttcatcctctacaaggatggggatgttgacagtcctgtggtccgtgaacggatctggcaaaacagtgatttcaacttcgacaacgtcctctctgctatgatggcgctcttcacagtctccacgtttgagggctggcctgcgttgctgtataaagccatcgactcgaatggagagaacatcggcccaatctacaaccaccgcgtggagatctccatcttcttcatcatctacatcatcattgtagctttcttcatgatgaacatctttgtgggctttgtcatcgttacatttcaggaacaaggagaaaaagagtataagaactgtgagctggacaaaaatcagcgtcagtgtgttgaatacgccttgaaagcacgtcccttgcggagatacatccccaaaaacccctaccagtacaagttctggtacgtggtgaactcttcgcctttcgaatacatgatgtttgtcctcatcatgctcaacacactctgcttggccatgcagcactacgagcagtccaagatgttcaatgatgccatggacattctgaacatggtcttcaccggggtgttcaccgtcgagatggttttgaaagtcatcgcatttaagcctaaggggtattttagtgacgcctggaacacgtttgactccctcatcgtaatcggcagcattatagacgtggccctcagcgaagcagacaactctgaagagagcaatagaatctccatcacctttttccgtcttttccgagtgatgcgattggtgaagcttctcagcaggggggaaggcatccggacattgctgtggacttttattaagtcctttcaggcgctcccgtatgtggccctcctcatagccatgctgttcttcatctatgcggtcattggcatgcagatgtttgggaaagttgccatgagagataacaaccagatcaataggaacaataacttccagacgtttccccaggcggtgctgctgctcttcaggtgtgcaacaggtgaggcctggcaggagatcatgctggcctgtctcccagggaagctctgtgaccctgagtcagattacaaccccggggaggagtatacatgtgggagcaactttgccattgtctatttcatcagtttttacatgctctgtgcatttctgatcatcaatctgtttgtggctgtcatcatggataatttcgactatctgacccgggactggtctattttggggcctcaccatttagatgaattcaaaagaatatggtcagaatatgaccctgaggcaaagggaaggataaaacaccttgatgtggtcactctgcttcgacgcatccagcctcccctggggtttgggaagttatgtccacacagggtagcgtgcaagagattagttgccatgaacatgcctctcaacagtgacgggacagtcatgtttaatgcaaccctgtttgctttggttcgaacggctcttaagatcaagaccgaagggaacctggagcaagctaatgaagaacttcgggctgtgataaagaaaatttggaagaaaaccagcatgaaattacttgaccaagttgtccctccagctggtgatgacgaggtaaccgtggggaagttctatgccactttcctgatacaggactactttaggaaattcaagaaacggaaagaacaaggactggtgggaaagtaccctgcgaagaacaccacaattgccctacaggcgggattaaggacactgcatgacattgggccagaaatccggcgtgctatatcgtgtgatttgcaagatgacgagcctgaggaaacaaaacgagaagaagaagatgatgtgttcaaaagaaatggtgccctgcttggaaaccatgtcaatcatgttaatagtgataggagagattcccttcagcagaccaataccacccaccgtcccctgcatgtccaaaggccttcaattccacctgcaagtgatactgagaaaccgctgtttcctccagcaggaaattcggtgtgtcataaccatcataaccataattccataggaaagcaagttcccacctcaacaaatgccaatctcaataatgccaatatgtccaaagctgcccatggaaagcggcccagcattgggaaccttgagcatgtgtctgaaaatgggcatcattcttcccacaagcatgaccgggagcctcagagaaggtccagtgtgaaaaggtaa

SEQ ID NO: 18 - DNA, 6137 bp, Homo sapiens
  modified_base (5946): a, t, c , g, unknown or other
  modified_base (5958): a, t, c , g, unknown or other
  modified_base (5983): a, t, c , g, unknown or other
  modified_base (5989): a, t, c , g, unknown or other
  modified_base (6000): a, t, c , g, unknown or other
  modified_base (6012): a, t, c , g, unknown or other
  modified_base (6047): a, t, c , g, unknown or other
  modified_base (6049): a, t, c , g, unknown or other
  modified_base (6051): a, t, c , g, unknown or other
  modified_base (6057): a, t, c , g, unknown or other
  modified_base (6062): a, t, c , g, unknown or other
  modified_base (6082): a, t, c , g, unknown or other
gaagcttttatactgcaacaaacaatacttgtgctttccctccatctagcactgtgcctccaggagagagggtttttttctggtcgcccctgcagacatgaccctgcttgtccctggccaatcccagggccagactggattgttggccctctgactcaccctagaaggcctggggctggagaggcctggaactcctcggagagttgagtgctccaaacagaatcaaactccaatgaataacaagtaattttggaatgttttacatcatgatggaaccgctgtttaaatgcaggaaaacaagctccaggctcccactgattctacatgatgaggcaaactatgcaagaggcaccagacttcctctttctggtgaaggaccaacttctcagccgaatagctccaagcaaactgtcctgtcttggcaagctgcaatcgatgctgctagacaggccaaggctgcccaaactatgagcacctctgcacccccacctgtaggatctctctcccaaagaaaacgtcagcaatacgccaagagcaaaaaacagggtaactcgtccaacagccgacctgcccgcgcccttttctgtttatcactcaataaccccatccgaagagcctgcattagtatagtggaatggaaaccatttgacatatttatattattggctatttttgccaattgtgtggccttagctatttacatcccattccctgaagatgattctaattcaacaaatcataacttggaaaaagtagaatatgccttcctgattatttttacagtcgagacatttttgaagattatagcgtatggattattgctacatcctaatgcttatgttaggaatggatggaatttactggattttgttatagtaatagtaggattgtttagtgtaattttggaacaattaaccaaagaaacagaaggcgggaaccactcaagcggcaaatctggaggctttgatgtcaaagccctccgtgcctttcgagtgttgcgaccacttcgactagtgtcaggggtgcccagtttacaagttgtcctgaactccattataaaagccatggttcccctccttcacatagcccttttggtattatttgtaatcataatctatgctattataggattggaactttttattggaaaaatgcacaaaacatgtttttttgctgactcagatatcgtagctgaagaggacccagctccatgtgcgttctcagggaatggacgccagtgtactgccaatggcacggaatgtaggagtggctgggttggcccgaacggaggcatcaccaactttgataactttgcctttgccatgcttactgtgtttcagtgcatcaccatggagggctggacagacgtgctctactgggtaaatgatgcgataggatgggaatggccatgggtgtattttgttagtctgatcatccttggctcatttttcgtccttaacctggttcttggtgtccttagtggagaattctcaaaggaaagagagaaggcaaaagcacggggagatttccagaagctccgggagaagcagcagctggaggaggatctaaagggctacttggattggatcacccaagctgaggacatcgatccggagaatgaggaagaaggaggagaggaaggcaaacgaaatactagcatgcccaccagcgagactgagtctgtgaacacagagaacgtcagcggtgaaggcgagaaccgaggctgctgtggaagtctctgtcaagccatctcaaaatccaaactcagccgacgctggcgtcgctggaaccgattcaatcgcagaagatgtagggccgccgtgaagtctgtcacgttttactggctggttatcgtcctggtgtttctgaacaccttaaccatttcctctgagcactacaatcagccagattggttgacacagattcaagatattgccaacaaagtcctcttggctctgttcacctgcgagatgctggtaaaaatgtacagcttgggcctccaagcatatttcgtctctcttttcaaccggtttgattgcttcgtggtgtgtggtggaatcactgagacgatcttggtggaactggaaatcatgtctcccctggggatctctgtgtttcggtgtgtgcgcctcttaagaatcttcaaagtgaccaggcactggacttccctgagcaacttagtggcatccttattaaactccatgaagtccatcgcctcgctgttgcttctgctttttctcttcattatcatcttttccttgcttgggatgcagctgtttggcggcaagtttaattttgatgaaacgcaaaccaagcggagcacctttgacaatttccctcaagcacttctcacagtgttccagatcctgacaggcgaagactggaatgctgtgatgtacgatggcatcatggcttacgggggcccatcctcttcaggaatgatcgtctgcatctacttcatcatcctcttcatttgtggtaactatattctactgaatgtcttcttggccatcgctgtagacaatttggctgatgctgaaagtctgaacactgctcagaaagaagaagcggaagaaaaggagaggaaaaagattgccagaaaagagagcctagaaaataaaaagaacaacaaaccagaagtcaaccagatagccaacagtgacaacaaggttacaattgatgactatagagaagaggatgaagacaaggacccctatccgccttgcgatgtgccagtaggggaagaggaagaggaagaggaggaggatgaacctgaggttcctgccggaccccgtcctcgaaggatctcggagttgaacatgaaggaaaaaattgcccccatccctgaagggagcgctttcttcattcttagcaagaccaacccgatccgcgtaggctgccacaagctcatcaaccaccacatcttcaccaacctcatccttgtcttcatcatgctgagcagcgctgccctggccgcagaggaccccatccgcagccactccttccggaacacgatactgggttactttgactatgccttcacagccatctttactgttgagatcctgttgaagatgacaacttttggagctttcctccacaaaggggccttctgcaggaactacttcaatttgctggatatgctggtggttggggtgtctctggtgtcatttgggattcaatccagtgccatctccgttgtgaagattctgagggtcttaagggtcctgcgtcccctcagggccatcaacagagcaaaaggacttaagcacgtggtccagtgcgtcttcgtggccatccggaccatcggcaacatcatgatcgtcaccaccctcctgcagttcatgtttgcctgtatcggggtccagttgttcaaggggaagttctatcgctgtacggatgaagccaaaagtaaccctgaagaatgcaggggacttttcatcctctacaaggatggggatgttgacagtcctgtggtccgtgaacggatctggcaaaacagtgatttcaacttcgacaacgtcctctctgctatgatggcgctcttcacagtctccacgtttgagggctggcctgcgttgctgtataaagccatcgactcgaatggagagaacatcggcccaatctacaaccaccgcgtggagatctccatcttcttcatcatctacatcatcattgtagctttcttcatgatgaacatctttgtgggctttgtcatcgttacatttcaggaacaaggagaaaaagagtataagaactgtgagctggacaaaaatcagcgtcagtgtgttgaatacgccttgaaagcacgtcccttgcggagatacatccccaaaaacccctaccagtacaagttctggtacgtggtgaactcttcgcctttcgaatacatgatgtttgtcctcatcatgctcaacacactctgcttggccatgcagcactacgagcagtccaagatgttcaatgatgccatggacattctgaacatggtcttcaccggggtgttcaccgtcgagatggttttgaaagtcatcgcatttaagcctaaggggtattttagtgacgcctggaacacgtttgactccctcatcgtaatcggcagcattatagacgtggccctcagcgaagcagacaactctgaagagagcaatagaatctccatcacctttttccgtcttttccgagtgatgcgattggtgaagcttctcagcaggggggaaggcatccggacattgctgtggacttttattaagtcctttcaggcgctcccgtatgtggccctcctcatagccatgctgttcttcatctatgcggtcattggcatgcagatgtttgggaaagttgccatgagagataacaaccagatcaataggaacaataacttccagacgtttccccaggcggtgctgctgctcttcaggtgtgcaacaggtgaggcctggcaggagatcatgctggcctgtctcccagggaagctctgtgaccctgagtcagattacaaccccggggaggagtatacatgtgggagcaactttgccattgtctatttcatcagtttttacatgctctgtgcatttctgatcatcaatctgtttgtggctgtcatcatggataatttcgactatctgacccgggactggtctattttggggcctcaccatttagatgaattcaaaagaatatggtcagaatatgaccctgaggcaaagggaaggataaaacaccttgatgtggtcactctgcttcgacgcatccagcctcccctggggtttgggaagttatgtccacacagggtagcgtgcaagagattagttgccatgaacatgcctctcaacagtgacgggacagtcatgtttaatgcaaccctgtttgctttggttcgaacggctcttaagatcaagaccgaagggaacctggagcaagctaatgaagaacttcgggctgtgataaagaaaatttggaagaaaaccagcatgaaattacttgaccaagttgtccctccagctggtgatgacgaggtaaccgtggggaagttctatgccactttcctgatacaggactactttaggaaattcaagaaacggaaagaacaaggactggtgggaaagtaccctgcgaagaacaccacaattgccctacaggcgggattaaggacactgcatgacattgggccagaaatccggcgtgctatatcgtgtgatttgcaagatgacgagcctgaggaaacaaaacgagaagaagaagatgatgtgttcaaaagaaatggtgccctgcttggaaaccatgtcaatcatgttaatagtgataggagagattcccttcagcagaccaataccacccaccgtcccctgcatgtccaaaggccttcaattccacctgcaagtgatactgagaaaccgctgtttcctccagcaggaaattcggtgtgtcataaccatcataaccataattccataggaaagcaagttcccacctcaacaaatgccaatctcaataatgccaatatgtccaaagctgcccatggaaagcggcccagcattgggaaccttgagcatgtgtctgaaaatgggcatcattcttcccacaagcatgaccgggagcctcagagaaggtccagtgtgaaaaggtaaccttgacaatgtgtttggacttgctcatgtggtgtctgcccgtgttgcgtctagtcccaaggagcaaggcgggtctgtgctctgtgctctggggcatggaggaggttccccgtaggcatttgtgtcaggatctgtgagtgccccccaggtcactgctgcctctgacactctccacgtgatggacctaaaggataagggccacgtgccatcctacatcctgtgtggggtgccgagggaccttwaaacagcagcagccacaaaatccacctctcaattgtgctcacatactgtgcgacaggggttggtgaattgtttntttaaagggtcnctgagtcaatattttaggctttganaaccanacagttttgcnccttttcagctntgccgttgcagtgtgaaatatgtaagtgaatgggngngnggctgngcccnttaaaatttccctatggtcnctgaaatttgaacttcaaaaaaaaaaaaaaaaaaaaaaaaaaaaaaaaaaaaaaa

SEQ ID NO: 19 - skipped

SEQ ID NO: 20 - DNA, 5460 bp, Homo sapiens
atgttttacatcatgatggaaccgctgtttaaatgcaggaaaacaagctccaggctcccactgattctacatgatgaggcaaactatgcaagaggcaccagacttcctctttctggtgaaggaccaacttctcagccgaatagctccaagcaaactgtcctgtcttggcaagctgcaatcgatgctgctagacaggccaaggctgcccaaactatgagcacctctgcacccccacctgtaggatctctctcccaaagaaaacgtcagcaatacgccaagagcaaaaaacagggtaactcgtccaacagccgacctgcccgcgcccttttctgtttatcactcaataaccccatccgaagagcctgcattagtatagtggaatggaaaccatttgacatatttatattattggctatttttgccaattgtgtggccttagctatttacatcccattccctgaagatgattctaattcaacaaatcataacttggaaaaagtagaatatgccttcctgattatttttacagtcgagacatttttgaagattatagcgtatggattattgctacatcctaatgcttatgttaggaatggatggaatttactggattttgttatagtaatagtaggattgtttagtgtaattttggaacaattaaccaaagaaacagaaggcgggaaccactcaagcggcaaatctggaggctttgatgtcaaagccctccgtgcctttcgagtgttgcgaccacttcgactagtgtcaggggtgcccagtttacaagttgtcctgaactccattataaaagccatggttcccctccttcacatagcccttttggtattatttgtaatcataatctatgctattataggattggaactttttattggaaaaatgcacaaaacatgtttttttgctgactcagatatcgtagctgaagaggacccagctccatgtgcgttctcagggaatggacgccagtgtactgccaatggcacggaatgtaggagtggctgggttggcccgaacggaggcatcaccaactttgataactttgcctttgccatgcttactgtgtttcagtgcatcaccatggagggctggacagacgtgctctactgggtaaatgatgcgataggatgggaatggccatgggtgtattttgttagtctgatcatccttggctcatttttcgtccttaacctggttcttggtgtccttagtggagaattctcaaaggaaagagagaaggcaaaagcacggggagatttccagaagctccgggagaagcagcagctggaggaggatctaaagggctacttggattggatcacccaagctgaggacatcgatccggagaatgaggaagaaggaggagaggaaggcaaacgaaatactagcatgcccaccagcgagactgagtctgtgaacacagagaacgtcagcggtgaaggcgagaaccgaggctgctgtggaagtctctgtcaagccatctcaaaatccaaactcagccgacgctggcgtcgctggaaccgattcaatcgcagaagatgtagggccgccgtgaagtctgtcacgttttactggctggttatcgtcctggtgtttctgaacaccttaaccatttcctctgagcactacaatcagccagattggttgacacagattcaagatattgccaacaaagtcctcttggctctgttcacctgcgagatgctggtaaaaatgtacagcttgggcctccaagcatatttcgtctctcttttcaaccggtttgattgcttcgtggtgtgtggtggaatcactgagacgatcttggtggaactggaaatcatgtctcccctggggatctctgtgtttcggtgtgtgcgcctcttaagaatcttcaaagtgaccaggcactggacttccctgagcaacttagtggcatccttattaaactccatgaagtccatcgcctcgctgttgcttctgctttttctcttcattatcatcttttccttgcttgggatgcagctgtttggcggcaagtttaattttgatgaaacgcaaaccaagcggagcacctttgacaatttccctcaagcacttctcacagtgttccagatcctgacaggcgaagactggaatgctgtgatgtacgatggcatcatggcttacgggggcccatcctcttcaggaatgatcgtctgcatctacttcatcatcctcttcatttgtggtaactatattctactgaatgtcttcttggccatcgctgtagacaatttggctgatgctgaaagtctgaacactgctcagaaagaagaagcggaagaaaaggagaggaaaaagattgccagaaaagagagcctagaaaataaaaagaacaacaaaccagaagtcaaccagatagccaacagtgacaacaaggttacaattgatgactatagagaagaggatgaagacaaggacccctatccgccttgcgatgtgccagtaggggaagaggaagaggaagaggaggaggatgaacctgaggttcctgccggaccccgtcctcgaaggatctcggagttgaacatgaaggaaaaaattgcccccatccctgaagggagcgctttcttcattcttagcaagaccaacccgatccgcgtaggctgccacaagctcatcaaccaccacatcttcaccaacctcatccttgtcttcatcatgctgagcagcgctgccctggccgcagaggaccccatccgcagccactccttccggaacacgatactgggttactttgactatgccttcacagccatctttactgttgagatcctgttgaagatgacaacttttggagctttcctccacaaaggggccttctgcaggaactacttcaatttgctggatatgctggtggttggggtgtctctggtgtcatttgggattcaatccagtgccatctccgttgtgaagattctgagggtcttaagggtcctgcgtcccctcagggccatcaacagagcaaaaggacttaagcacgtggtccagtgcgtcttcgtggccatccggaccatcggcaacatcatgatcgtcaccaccctcctgcagttcatgtttgcctgtatcggggtccagttgttcaaggggaagttctatcgctgtacggatgaagccaaaagtaaccctgaagaatgcaggggacttttcatcctctacaaggatggggatgttgacagtcctgtggtccgtgaacggatctggcaaaacagtgatttcaacttcgacaacgtcctctctgctatgatggcgctcttcacagtctccacgtttgagggctggcctgcgttgctgtataaagccatcgactcgaatggagagaacatcggcccaatctacaaccaccgcgtggagatctccatcttcttcatcatctacatcatcattgtagctttcttcatgatgaacatctttgtgggctttgtcatcgttacatttcaggaacaaggagaaaaagagtataagaactgtgagctggacaaaaatcagcgtcagtgtgttgaatacgccttgaaagcacgtcccttgcggagatacatccccaaaaacccctaccagtacaagttctggtacgtggtgaactcttcgcctttcgaatacatgatgtttgtcctcatcatgctcaacacactctgcttggccatgcagcactacgagcagtccaagatgttcaatgatgccatggacattctgaacatggtcttcaccggggtgttcaccgtcgagatggttttgaaagtcatcgcatttaagcctaaggggtattttagtgacgcctggaacacgtttgactccctcatcgtaatcggcagcattatagacgtggccctcagcgaagcagaccactatttcactgatgcatggaacacttttgatgccttaattgttgttggtagcgtcgttgatattgctataactgaagtgaataactctgaagagagcaatagaatctccatcacctttttccgtcttttccgagtgatgcgattggtgaagcttctcagcaggggggaaggcatccggacattgctgtggacttttattaagtcctttcaggcgctcccgtatgtggccctcctcatagccatgctgttcttcatctatgcggtcattggcatgcagatgtttgggaaagttgccatgagagataacaaccagatcaataggaacaataacttccagacgtttccccaggcggtgctgctgctcttcaggtgtgcaacaggtgaggcctggcaggagatcatgctggcctgtctcccagggaagctctgtgaccctgagtcagattacaaccccggggaggagtatacatgtgggagcaactttgccattgtctatttcatcagtttttacatgctctgtgcatttctgatcatcaatctgtttgtggctgtcatcatggataatttcgactatctgacccgggactggtctattttggggcctcaccatttagatgaattcaaaagaatatggtcagaatatgaccctgaggcaaagggaaggataaaacaccttgatgtggtcactctgcttcgacgcatccagcctcccctggggtttgggaagttatgtccacacagggtagcgtgcaagagattagttgccatgaacatgcctctcaacagtgacgggacagtcatgtttaatgcaaccctgtttgctttggttcgaacggctcttaagatcaagaccgaagggaacctggagcaagctaatgaagaacttcgggctgtgataaagaaaatttggaagaaaaccagcatgaaattacttgaccaagttgtccctccagctggtgatgacgaggtaaccgtggggaagttctatgccactttcctgatacaggactactttaggaaattcaagaaacggaaagaacaaggactggtgggaaagtaccctgcgaagaacaccacaattgccctacaggcgggattaaggacactgcatgacattgggccagaaatccggcgtgctatatcgtgtgatttgcaagatgacgagcctgaggaaacaaaacgagaagaagaagatgatgtgttcaaaagaaatggtgccctgcttggaaaccatgtcaatcatgttaatagtgataggagagattcccttcagcagaccaataccacccaccgtcccctgcatgtccaaaggccttcaattccacctgcaagtgatactgagaaaccgctgtttcctccagcaggaaattcggtgtgtcataaccatcataaccataattccataggaaagcaagttcccacctcaacaaatgccaatctcaataatgccaatatgtccaaagctgcccatggaaagcggcccagcattgggaaccttgagcatgtgtctgaaaatgggcatcattcttcccacaagcatgaccgggagcctcagagaaggtccagtgtgaaaaggtaa

SEQ ID NO: 21 - protein, 1796 aa, Homo sapiens
MFYIMMEPLMMEPLFRCRKTSSRLPLILHDEANYARGTRLPLSGEGPTSQPNSSKQTVLSWQAAIDAARQAKAAQTMSTSAPPPVGSLSQRKRQQYAKSKKQGNSSNSRPARALFCLSLNNPIRRACISIVEWKPFDIFILLAIFANCVALAIYIPFPEDDSNSTNHNLEKVEYAFLIIFTVETFLKIIAYGLLLHPNAYVRNGWNLLDFVIVIVGLFSVILEQLTKETEGGNHSSGKSGGFDVKALRAFRVLRPLRLVSGVPSLQVVLNSIIKAMVPLLHIALLVLFVIIIYAIIGLELFIGKMHKTCFFADSDIVAEEDPAPCAFSGNGRQCTANGTECRSGWVGPNGGITNFDNFAFAMLTVFQCITMEGWTDVLYWVNDAIGWEWPWVYFVSLIILGSFFVLNLVLGVLSGEFSKEREKAKARGDFQKLREKQQLEEDLKGYLDWITQAEDIDPENEEEGGEEGKRNTSMPTSETESVNTENVSGEGENRGCCGSLCQAISKSKLSRRWRRWNRFNRRRCRAAVKSVTFYWLVIVLVFLNTLTISSEHYNQPDWLTQIQDIANKVLLALFTCEMLVKMYSLGLQAYFVSLFNRFDCFVVCGGITETILVELEIMSPLGISVFRCVRLLRIFKVTRHWTSLSNLVASLLNSMKSIASLLLLLFLFIIIFSLLGMQLFGGKFNFDETQTKRSTFDNFPQALLTVFQILTGEDWNAVMYDGIMAYGGPSSSGMIVCIYFIILFICGNYILLNVFLAIAVDNLADAESLNTAQKEEAEEKERKKIARKESLENKKNNKPEVNQIANSDNKVTIDDYREEDEDKDPYPPCDVPVGEEEEEEEEDEPEVPAGPRPRRISELNMKEKIAPIPEGSAFFILSKTNPIRVGCHKLINHHIFTNLILVFIMLSSAALAAEDPIRSHSFRNTILGYFDYAFTAIFTVEILLKMTTFGAFLHKGAFCRNYFNLLDMLVVGVSLVSFGIQSSAISVVKILRVLRVLRPLRAINRAKGLKHVVQCVFVAIRTIGNIMIVTTLLQFMFACIGVQLFKGKFYRCTDEAKSNPEECRGLFILYKDGDVDSPVVRERIWQNSDFNFDNVLSAMMALFTVSTFEGWPALLYKAIDSNGENIGPIYNHRVEISIFFIIYIIIVAFFMMNIFVGFVIVTFQEQGEKEYKNCELDKNQRQCVEYALKARPLRRYIPKNPYQYKFWYVVNSSPFEYMMFVLIMLNTLCLAMQHYEQSKMFNDAMDILNMVFTGVFTVEMVLKVIAFKPKGYFSDAWNTFDSLIVIGSIIDVALSEADNSEESNRISITFFRLFRVMRLVKLLSRGEGIRTLLWTFIKSFQALPYVALLIAMLFFIYAVIGMQMFGKVAMRDNNQINRNNNFQTFPQAVLLLFRCATGEAWQEIMLACLPGKLCDPESDYNPGEEYTCGSNFAIVYFISFYMLCAFLIINLFVAVIMDNFDYLTRDWSILGPHHLDEFKRIWSEYDPEAKGRIKHLDVVTLLRRIQPPLGFGKLCPHRVACKRLVAMNMPLNSDGTVMFNATLFALVRTALKIKTEGNLEQANEELRAVIKKIWKKTSMKLLDQVVPPAGDDEVTVGKFYATFLIQDYFRKFKKRKEQGLVGKYPAKNTTIALQAGLRTLHDIGPEIRRAISCDLQDDEPEETKREEEDDVFKRNGALLGNHVNHVNSDRRDSLQQTNTTHRPLHVQRPSIPPASDTEKPLFPPAGNSVCHNHHNHNSIGKQVPTSTNANLNNANMSKAAHGKRPSIGNLEHVSENGHHSSHKHDREPQRRSSVKR

SEQ ID NO: 22 - protein, 1815 aa, Homo sapiens
MMEPLFRCRKTSSRLPLILHDEANYARGTRLPLSGEGPTSQPNSSKQTVLSWQAAIDAARQAKAAQTMSTSAPPPVGSLSQRKRQQYAKSKKQGNSSNSRPARALFCLSLNNPIRRACISIVEWKPFDIFILLAIFANCVALAIYIPFPEDDSNSTNHNLEKVEYAFLIIFTVETFLKIIAYGLLLHPNAYVRNGWNLLDFVIVIVGLFSVILEQLTKETEGGNHSSGKSGGFDVKALRAFRVLRPLRLVSGVPSLQVVLNSIIKAMVPLLHIALLVLFVIIIYAIIGLELFIGKMHKTCFFADSDIVAEEDPAPCAFSGNGRQCTANGTECRSGWVGPNGGITNFDNFAFAMLTVFQCITMEGWTDVLYWVNDAIGWEWPWVYFVSLIILGSFFVLNLVLGVLSGEFSKEREKAKARGDFQKLREKQQLEEDLKGYLDWITQAEDIDPENEEEGGEEGKRNTSMPTSETESVNTENVSGEGENRGCCGSLCQAISKSKLSRRWRRWNRFNRRRCRAAVKSVTFYWLVIVLVFLNTLTISSEHYNQPDWLTQIQDIANKVLLALFTCEMLVKMYSLGLQAYFVSLFNRFDCFVVCGGITETILVELEIMSPLGISVFRCVRLLRIFKVTRHWTSLSNLVASLLNSMKSIASLLLLLFLFIIIFSLLGMQLFGGKFNFDETQTKRSTFDNFPQALLTVFQILTGEDWNAVMYDGIMAYGGPSSSGMIVCIYFIILFICGNYILLNVFLAIAVDNLADAESLNTAQKEEAEEKERKKIARKESLENKKNNKPEVNQIANSDNKVTIDDYREEDEDKDPYPPCDVPVGEEEEEEEEDEPEVPAGPRPRRISELNMKEKIAPIPEGSAFFILSKTNPIRVGCHKLINHHIFTNLILVFIMLSSAALAAEDPIRSHSFRNTILGYFDYAFTAIFTVEILLKMTTFGAFLHKGAFCRNYFNLLDMLVVGVSLVSFGIQSSAISVVKILRVLRVLRPLRAINRAKGLKHVVQCVFVAIRTIGNIMIVTTLLQFMFACIGVQLFKGKFYRCTDEAKSNPEECRGLFILYKDGDVDSPVVRERIWQNSDFNFDNVLSAMMALFTVSTFEGWPALLYKAIDSNGENIGPIYNHRVEISIFFIIYIIIVAFFMMNIFVGFVIVTFQEQGEKEYKNCELDKNQRQCVEYALKARPLRRYIPKNPYQYKFWYVVNSSPFEYMMFVLIMLNTLCLAMQHYEQSKMFNDAMDILNMVFTGVFTVEMVLKVIAFKPKGYFSDAWNTFDSLIVIGSIIDVALSEADHYFTDAWNTFDALIVVGSVVDIAITEVNNSEESNRISITFFRLFRVMRLVKLLSRGEGIRTLLWTFIKSFQALPYVALLIAMLFFIYAVIGMQMFGKVAMRDNNQINRNNNFQTFPQAVLLLFRCATGEAWQEIMLACLPGKLCDPESDYNPGEEYTCGSNFAIVYFISFYMLCAFLIINLFVAVIMDNFDYLTRDWSILGPHHLDEFKRIWSEYDPEAKGRIKHLDVVTLLRRIQPPLGFGKLCPHRVACKRLVAMNMPLNSDGTVMFNATLFALVRTALKIKTEGNLEQANEELRAVIKKIWKKTSMKLLDQVVPPAGDDEVTVGKFYATFLIQDYFRKFKKRKEQGLVGKYPAKNTTIALQAGLRTLHDIGPEIRRAISCDLQDDEPEETKREEEDDVFKRNGALLGNHVNHVNSDRRDSLQQTNTTHRPLHVQRPSIPPASDTEKPLFPPAGNSVCHNHHNHNSIGKQVPTSTNANLNNANMSKAAHGKRPSIGNLEHVSENGHHSSHKHDREPQRRSSVKR

SEQ ID NO: 23 - DNA, 86 bp, Homo sapiens
atgccagtgcctggccatgacgtggaggcctactgcctgctgtgcgagtgcaggtacgaggagcgcagcaccaccaccatcaaggt

SEQ ID NO: 24 - protein, 29 aa, Homo sapiens
MPVPGHDVEAYCLLCECRYEERSTTTIKV

SEQ ID NO: 25 - DNA, 129 bp, Homo sapiens
catcattgtcatctacctgtccgtggtgggtgccctgttgctctacatggccttcctgatgctggtggaccctctgatccgaaagccggatgcatacactgagcaactgcacaatgaggaggagaatga

SEQ ID NO: 26 - DNA, 215 bp, Homo sapiens
atgccagtgcctggccatgacgtggaggcctactgcctgctgtgcgagtgcaggtacgaggagcgcagcaccaccaccatcaaggtcatcattgtcatctacctgtccgtggtgggtgccctgttgctctacatggccttcctgatgctggtggaccctctgatccgaaagccggatgcatacactgagcaactgcacaatgaggaggagaatga

SEQ ID NO: 27 - protein, 72 aa, Homo sapiens
MPVPGHDVEAYCLLCECRYEERSTTTIKVIIVIYLSVVGALLLYMAFLMLVDPLIRKPDAYTEQLHNEEENE

SEQ ID NO: 28 - protein, 43 aa, Homo sapiens
IIVIYLSVVGALLLYMAFLMLVDPLIRKPDAYTEQLHNEEENE

SEQ ID NO: 29 - DNA, 27 bp, Artificial Sequence
  Description of Artificial Sequence: Primer
ccctgttttttgctcttggcgtattgc

SEQ ID NO: 30 - DNA, 27 bp, Artificial Sequence
  Description of Artificial Sequence: Primer
cttggcgtattgctgacgttttctttg

SEQ ID NO: 31 - protein, 25 aa, Homo sapiens
MFYIMMEPLFRCRKTSSRLPLILHD

SEQ ID NO: 32 - protein, 21 aa, Homo sapiens
MMEPLFRCRKTSSRLPLILHD

SEQ ID NO: 33 - protein, 25 aa, Homo sapiens
MFYIMMEPLFRCRKTSSRLPLILHD

SEQ ID NO: 34 - DNA, 26 bp, Artificial Sequence
  Description of Artificial Sequence: Primer
caggctcttcggatggggttattgag

SEQ ID NO: 35 - protein, 28 aa, Homo sapiens
MFYIMEPLFRCRKTSSRLPLILHDEANY

SEQ ID NO: 36 - protein, 21 aa, Homo sapiens
MMEPLFRCRKTSSRLPLILHD

SEQ ID NO: 37 - DNA, 28 bp, Artificial Sequence
  Description of Artificial Sequence: Primer
caagttcccacctcaacaaatgccaatc

SEQ ID NO: 38 - DNA, 27 bp, Artificial Sequence
  Description of Artificial Sequence: Primer
acaaatgccaatctcaataatgccaat

SEQ ID NO: 39 - DNA, 510 bp, Homo sapiens
  modified_base (319): a, t, c, g, unknown or other
  modified_base (331): a, t, c, g, unknown or other
  modified_base (356): a, t, c, g, unknown or other
  modified_base (362): a, t, c, g, unknown or other
  modified_base (373): a, t, c, g, unknown or other
  modified_base (385): a, t, c, g, unknown or other
  modified_base (420): a, t, c, g, unknown or other
  modified_base (422): a, t, c, g, unknown or other
  modified_base (424): a, t, c, g, unknown or other
  modified_base (430): a, t, c, g, unknown or other
  modified_base (435): a, t, c, g, unknown or other
  modified_base (455): a, t, c, g, unknown or other
taaccttgacaatgtgtttggacttgctcatgtggtgtctgcccgtgttgcgtctagtcccaaggagcaaggcgggtctgtgctctgtgctctggggcatggaggaggttccccgtaggcatttgtgtcaggatctgtgagtgccccccaggtcactgctgcctctgacactctccacgtgatggacctaaaggataagggccacgtgccatcctacatcctgtgtggggtgccgagggaccttwaaacagcagcagccacaaaatccacctctcaattgtgctcacatactgtgcgacaggggttggtgaattgtttntttaaagggtcnctgagtcaatattttaggctttganaaccanacagttttgcnccttttcagctntgccgttgcagtgtgaaatatgtaagtgaatgggngngnggctgngcccnttaaaatttccctatggtcnctgaaatttgaacttcaaaaaaaaaaaaaaaaaaaaaaaaaaaaaaaaaaaaaaa

SEQ ID NO: 40 - DNA, 517 bp, Homo sapiens
  modified_base (273): a, t, c, g, unknown or other
  modified_base (281): a, t, c, g, unknown or other
  modified_base (283): a, t, c, g, unknown or other
  modified_base (286): a, t, c, g, unknown or other
  modified_base (289): a, t, c, g, unknown or other
  modified_base (319): a, t, c, g, unknown or other
  modified_base (356): a, t, c, g, unknown or other
  modified_base (362): a, t, c, g, unknown or other
  modified_base (387): a, t, c, g, unknown or other
  modified_base (400): a, t, c, g, unknown or other
  modified_base (420): a, t, c, g, unknown or other
  modified_base (422): a, t, c, g, unknown or other
  modified_base (424): a, t, c, g, unknown or other
  modified_base (435): a, t, c, g, unknown or other
  modified_base (472): a, t, c, g, unknown or other
taaccttgacaatgtgtttggacttgctcatgtggtgtctgcccgtgttgcgtctagtcccaaggagcaaggtgggtctgtgctctgtgctctggggcatggaggaggttccccgtaggcatttgtgtcaggatctgtgagtgccccccaggtcactgctgcctctgacactctccacgtgatggacctaaaggataagggccacgtgccatcctacatcctgtgtggggtgccgagggaccttaaaacagcagcagccmcaaaatccmcctntcaattgngntcncanactgtgcgacaggggttggtgaattgtttntttaaagggtccctgagtcaatattttaggctttganaaccanacagttttgccccttttcagctctnccgttgcagtgtnaaatatgtaagtgaatgggngngnggctgtgcccnttaaaatttccctatggtccctgaaatttgaacttcnaaaaaaaaaaaaaaaaaaaaaaaaaaaaaaaaaaaaaaaaaaaaa

SEQ ID NO: 41 - DNA, 510 bp, Homo sapiens
  modified_base (319): a, t, c, g, unknown or other
  modified_base (321): a, t, c, g, unknown or other
  modified_base (331): a, t, c, g, unknown or other
  modified_base (362): a, t, c, g, unknown or other
  modified_base (368): a, t, c, g, unknown or other
  modified_base (373): a, t, c, g, unknown or other
  modified_base (422): a, t, c, g, unknown or other
  modified_base (424): a, t, c, g, unknown or other
  modified_base (430): a, t, c, g, unknown or other
  modified_base (455): a, t, c, g, unknown or other
taaccttgacaatgtgtttggacttgctcatgtggtgtctgcccgtgttgcgtctagtcccaaggagcaaggcgggtctgtgctctgtgctctggggcatggaggaggttccccgtaggcatttgtgtcaggatttgtgagtgccccccaggtcactgctgcctctgacactctccacgtgatggacctaaaggataagggccacgtgccatcctacatcctgtgtggggtgccgagggaccttaaaacagcagcagccacaaaatccacctctcaattgtgcttacatactgtgcgacaggggttggtgaattgtttntntaaagggtcnctgagtcaatattttaggctttgagaaccanacagtnttgcnccttttcagctctgccgttgcagtgtgaaatatgtaagtgaatgggtgngnggctgngcccattaaaatttccctatggtcnctgaaatttgaacttcgagaaaaaaaaaaaaaaaaaaaaaaaaaaaaaaaaaaaa

SEQ ID NO: 42 - DNA, 517 bp, Artificial Sequence
  Description of Artificial Sequence: Consensus sequence
  modified_base (319): a, t, c, g, unknown or other
  modified_base (331): a, t, c, g, unknown or other
  modified_base (356): a, t, c, g, unknown or other
  modified_base (362): a, t, c, g, unknown or other
  modified_base (373): a, t, c, g, unknown or other
  modified_base (420): a, t, c, g, unknown or other
  modified_base (422): a, t, c, g, unknown or other
  modified_base (430): a, t, c, g, unknown or other
  modified_base (435): a, t, c, g, unknown or other
  modified_base (455): a, t, c, g, unknown or other
taaccttgacaatgtgtttggacttgctcatgtggtgtctgcccgtgttgcgtctagtcccaaggagcaaggcgggtctgtgctctgtgctctggggcatggaggaggttccccgtaggcatttgtgtcaggatctgtgagtgccccccaggtcactgctgcctctgacactctccacgtgatggacctaaaggataagggccacgtgccatcctacatcctgtgtggggtgccgagggaccttaaaacagcagcagccacaaaatccacctctcaattgtgctcacatactgtgcgacaggggttggtgaattgtttntttaaagggtcnctgagtcaatattttaggctttganaaccanacagttttgcnccttttcagctctgccgttgcagtgtgaaatatgtaagtgaatgggngngtggctgngcccnttaaaatttccctatggtcnctgaaatttgaacttcraaaaaaaaaaaaaaaaaaaaaaaaaaaaaaaaaaaaaaaaaaaaa

SEQ ID NO: 43 - protein, 20 aa, Homo sapiens
CWWRRRGAAKAGPSGCRRWG

SEQ ID NO: 44 - protein, 15 aa, Homo sapiens
PTESENVPVPTATPG

SEQ ID NO: 45 - protein, 10 aa, Homo sapiens
RTRYYETYIR

SEQ ID NO: 46 - DNA, 29 bp, Artificial Sequence
  Description of Artificial Sequence: Primer
agtgttcagactttcagcatcagccaaat

SEQ ID NO: 47 - DNA, 27 bp, Artificial Sequence
  Description of Artificial Sequence: Primer
cagcatcagccaaattgtctacagcga

SEQ ID NO: 48 - DNA, 27 bp, Artificial Sequence
  Description of Artificial Sequence: Primer
cgatgactcgcccgtttgctatgattc

SEQ ID NO: 49 - DNA, 24 bp, Artificial Sequence
  Description of Artificial Sequence: Primer
tgctatgattcacggagatctcca

SEQ ID NO: 50 - DNA, 4920 bp, Homo sapiens
atgcccaccagcgagactgagtctgtgaacacagagaacgtcagcggtgaaggcgagaaccgaggctgctgtggaagtctctgtcaagccatctcaaaatccaaactcagccgacgctggcgtcgctggaaccgattcaatcgcagaagatgtagggccgccgtgaagtctgtcacgttttactggctggttatcgtcctggtgtttctgaacaccttaaccatttcctctgagcactacaatcagccagattggttgacacagattcaagatattgccaacaaagtcctcttggctctgttcacctgcgagatgctggtaaaaatgtacagcttgggcctccaagcatatttcgtctctcttttcaaccggtttgattgcttcgtggtgtgtggtggaatcactgagacgatcttggtggaactggaaatcatgtctcccctggggatctctgtgtttcggtgtgtgcgcctcttaagaatcttcaaagtgaccaggcactggacttccctgagcaacttagtggcatccttattaaactccatgaagtccatcgcctcgctgttgcttctgctttttctcttcattatcatcttttccttgcttgggatgcagctgtttggcggcaagtttaattttgatgaaacgcaaaccaagcggagcacctttgacaatttccctcaagcacttctcacagtgttccagatcctgacaggcgaagactggaatgctgtgatgtacgatggcatcatggcttacgggggcccatcctcttcaggaatgatcgtctgcatctacttcatcatcctcttcatttgtggtaactatattctactgaatgtcttcttggccatcgctgtagacaatttggctgatgctgaaagtctgaacactgctcagaaagaagaagcggaagaaaaggagaggaaaaagattgccagaaaagagagcctagaaaataaaaagaacaacaaaccagaagtcaaccagatagccaacagtgacaacaaggttacaattgatgactatagagaagaggatgaagacaaggacccctatccgccttgcgatgtgccagtaggggaagaggaagaggaagaggaggaggatgaacctgaggttcctgccggaccccgtcctcgaaggatctcggagttgaacatgaaggaaaaaattgcccccatccctgaagggagcgctttcttcattcttagcaagaccaacccgatccgcgtaggctgccacaagctcatcaaccaccacatcttcaccaacctcatccttgtcttcatcatgctgagcagcgctgccctggccgcagaggaccccatccgcagccactccttccggaacacgatactgggttactttgactatgccttcacagccatctttactgttgagatcctgttgaagatgacaacttttggagctttcctccacaaaggggccttctgcaggaactacttcaatttgctggatatgctggtggttggggtgtctctggtgtcatttgggattcaatccagtgccatctccgttgtgaagattctgagggtcttaagggtcctgcgtcccctcagggccatcaacagagcaaaaggacttaagcacgtggtccagtgcgtcttcgtggccatccggaccatcggcaacatcatgatcgtcaccaccctcctgcagttcatgtttgcctgtatcggggtccagttgttcaaggggaagttctatcgctgtacggatgaagccaaaagtaaccctgaagaatgcaggggacttttcatcctctacaaggatggggatgttgacagtcctgtggtccgtgaacggatctggcaaaacagtgatttcaacttcgacaacgtcctctctgctatgatggcgctcttcacagtctccacgtttgagggctggcctgcgttgctgtataaagccatcgactcgaatggagagaacatcggcccaatctacaaccaccgcgtggagatctccatcttcttcatcatctacatcatcattgtagctttcttcatgatgaacatctttgtgggctttgtcatcgttacatttcaggaacaaggagaaaaagagtataagaactgtgagctggacaaaaatcagcgtcagtgtgttgaatacgccttgaaagcacgtcccttgcggagatacatccccaaaaacccctaccagtacaagttctggtacgtggtgaactcttcgcctttcgaatacatgatgtttgtcctcatcatgctcaacacactctgcttggccatgcagcactacgagcagtccaagatgttcaatgatgccatggacattctgaacatggtcttcaccggggtgttcaccgtcgagatggttttgaaagtcatcgcatttaagcctaaggggtattttagtgacgcctggaacacgtttgactccctcatcgtaatcggcagcattatagacgtggccctcagcgaagcagacaactctgaagagagcaatagaatctccatcacctttttccgtcttttccgagtgatgcgattggtgaagcttctcagcaggggggaaggcatccggacattgctgtggacttttattaagtcctttcaggcgctcccgtatgtggccctcctcatagccatgctgttcttcatctatgcggtcattggcatgcagatgtttgggaaagttgccatgagagataacaaccagatcaataggaacaataacttccagacgtttccccaggcggtgctgctgctcttcaggtgtgcaacaggtgaggcctggcaggagatcatgctggcctgtctcccagggaagctctgtgaccctgagtcagattacaaccccggggaggagtatacatgtgggagcaactttgccattgtctatttcatcagtttttacatgctctgtgcatttctgatcatcaatctgtttgtggctgtcatcatggataatttcgactatctgacccgggactggtctattttggggcctcaccatttagtggggtttgggaagttatgtccacacagggtagcgtgcaagagattagttgccatgaacatgcctctcaacagtgacgggacagtcatgtttaatgcaaccctgtttgctttggttcgaacggctcttaagatcaagaccgaagggaacctggagcaagctaatgaagaacttcgggctgtgataaagaaaatttggaagaaaaccagcatgaaattacttgaccaagttgtccctccagctggtgatgacgaggtaaccgtggggaagttctatgccactttcctgatacaggactactttaggaaattcaagaaacggaaagaacaaggactggtgggaaagtaccctgcgaagaacaccacaattgccctacaggcgggattaaggacactgcatgacattgggccagaaatccggcgtgctatatcgtgtgatttgcaagatgacgagcctgaggaaacaaaacgagaagaagaagatgatgtgttcaaaagaaatggtgccctgcttggaaaccatgtcaatcatgttaatagtgataggagagattcccttcagcagaccaataccacccaccgtcccctgcatgtccaaaggccttcaattccacctgcaagtgatactgagaaaccgctgtttcctccagcaggaaattcggtgtgtcataaccatcataaccataattccataggaaagcaagttcccacctcaacaaatgccaatctcaataatgccaatatgtccaaagctgcccatggaaagcggcccagcattgggaaccttgagcatgtgtctgaaaatgggcatcattcttcccacaagcatgaccgggagcctcagagaaggtccagtgtgaaaaggtccgactcaggagatgaacagctcccaactatttgccgggaagacccagagatacatggctatttcagggacccccactgcttgggggagcaggagtatttcagtagtgaggaatgctacgaggatgacagctcgcccacctggagcaggcaaaactatggctactacagcagatacccaggcagaaacatcgactctgagaggccccgaggctaccatcatccccaaggattcttggaggacgatgactcgcccgtttgctatgattcacggagatctccaaggagacgcctactacctcccaccccagcatcccaccggagatcctccttcaactttgagtgcctgcgccggcagagcagccaggaagaggtcccgtcgtctcccatcttcccccatcgcacggccctgcctctgcatctaatgcagcaacagatcatggcagttgccggcctagattcaagtaaagcccagaagtactcaccgagtcactcgacccggtcgtgggccacccctccagcaacccctccctaccgggactggacaccgtgctacacccccctgatccaagtggagcagtcagaggccctggaccaggtgaacggcagcctgccgtccctgcaccgcagctcctggtacacagacgagcccgacatctcctaccggactttcacaccagccagcctgactgtccccagcagcttccggaacaaaaacagcgacaagcagaggagtgcggacagcttggtggaggcagtcctgatatccgaaggcttgggacgctatgcaagggacccaaaatttgtgtcagcaacaaaacacgaaatcgctgatgcctgtgacctcaccatcgacgagatggagagtgcagccagcaccctgcttaatgggaacgtgcgtccccgagccaacggggatgtgggccccctctcacaccggcaggactatgagctacaggactttggtcctggctacagcgacgaagagccagaccctgggagggatgaggaggacctggcggatgaaatgatatgcatcaccaccttgtag

SEQ ID NO: 51 - protein, 1639 aa, Homo sapiens
MPTSETESVNTENVSGEGENRGCCGSLCQAISKSKLSRRWRRWNRFNRRRCRAAVKSVTFYWLVIVLVFLNTLTISSEHYNQPDWLTQIQDIANKVLLALFTCEMLVKMYSLGLQAYFVSLFNRFDCFVVCGGITETILVELEIMSPLGISVFRCVRLLRIFKVTRHWTSLSNLVASLLNSMKSIASLLLLLFLFIIIFSLLGMQLFGGKFNFDETQTKRSTFDNFPQALLTVFQILTGEDWNAVMYDGIMAYGGPSSSGMIVCIYFIILFICGNYILLNVFLAIAVDNLADAESLNTAQKEEAEEKERKKIARKESLENKKNNKPEVNQIANSDNKVTIDDYREEDEDKDPYPPCDVPVGEEEEEEEEDEPEVPAGPRPRRISELNMKEKIAPIPEGSAFFILSKTNPIRVGCHKLINHHIFTNLILVFIMLSSAALAAEDPIRSHSFRNTILGYFDYAFTAIFTVEILLKMTTFGAFLHKGAFCRNYFNLLDMLVVGVSLVSFGIQSSAISVVKILRVLRVLRPLRAINRAKGLKHVVQCVFVAIRTIGNIMIVTTLLQFMFACIGVQLFKGKFYRCTDEAKSNPEECRGLFILYKDGDVDSPVVRERIWQNSDFNFDNVLSAMMALFTVSTFEGWPALLYKAIDSNGENIGPIYNHRVEISIFFIIYIIIVAFFMMNIFVGFVIVTFQEQGEKEYKNCELDKNQRQCVEYALKARPLRRYIPKNPYQYKFWYVVNSSPFEYMMFVLIMLNTLCLAMQHYEQSKMFNDAMDILNMVFTGVFTVEMVLKVIAFKPKGYFSDAWNTFDSLIVIGSIIDVALSEADNSEESNRISITFFRLFRVMRLVKLLSRGEGIRTLLWTFIKSFQALPYVALLIAMLFFIYAVIGMQMFGKVAMRDNNQINRNNNFQTFPQAVLLLFRCATGEAWQEIMLACLPGKLCDPESDYNPGEEYTCGSNFAIVYFISFYMLCAFLIINLFVAVIMDNFDYLTRDWSILGPHHLVGFGKLCPHRVACKRLVAMNMPLNSDGTVMFNATLFALVRTALKIKTEGNLEQANEELRAVIKKIWKKTSMKLLDQVVPPAGDDEVTVGKFYATFLIQDYFRKFKKRKEQGLVGKYPAKNTTIALQAGLRTLHDIGPEIRRAISCDLQDDEPEETKREEEDDVFKRNGALLGNHVNHVNSDRRDSLQQTNTTHRPLHVQRPSIPPASDTEKPLFPPAGNSVCHNHHNHNSIGKQVPTSTNANLNNANMSKAAHGKRPSIGNLEHVSENGHHSSHKHDREPQRRSSVKRSDSGDEQLPTICREDPEIHGYFRDPHCLGEQEYFSSEECYEDDSSPTWSRQNYGYYSRYPGRNIDSERPRGYHHPQGFLEDDDSPVCYDSRRSPRRRLLPPTPASHRRSSFNFECLRRQSSQEEVPSSPIFPHRTALPLHLMQQQIMAVAGLDSSKAQKYSPSHSTRSWATPPATPPYRDWTPCYTPLIQVEQSEALDQVNGSLPSLHRSSWYTDEPDISYRTFTPASLTVPSSFRNKNSDKQRSADSLVEAVLISEGLGRYARDPKFVSATKHEIADACDLTIDEMESAASTLLNGNVRPRANGDVGPLSHRQDYELQDFGPGYSDEEPDPGRDEEDLADEMICITTL

SEQ ID NO: 52 - protein, 47 aa, Homo sapiens
LFSVILEQLTKETEGGNHSSGKSGGFDVKALRAFRVLRPLRLVSGVP

SEQ ID NO: 53 - protein, 47 aa, Gallus gallus
LFSVILEQLTKETEGGSHSGGKPGGFDVKALRAFRVLRPLRLVSGVP

SEQ ID NO: 54 - protein, 47 aa, Artificial Sequence
  Description of Artificial Sequence: Consensus sequence
  MOD_RES (17): Ser or Asn
  MOD_RES (20): Ser or Gly
  MOD_RES (23): Ser or Pro
LFSVILEQLTKETEGGXHSXGKXGGFDVKALRAFRVLRPLRLVSGVP

SEQ ID NO: 55 - DNA, 27 bp, Artificial Sequence
  Description of Artificial Sequence: Primer
catatggattattattacaccccaatg

SEQ ID NO: 56 - DNA, 24 bp, Artificial Sequence
  Description of Artificial Sequence: Primer
cggagaggtcgcaatacacgaaag

SEQ ID NO: 57 - DNA, 26 bp, Artificial Sequence
  Description of Artificial Sequence: Primer
aagccctaagagcctttcgtgtattg

SEQ ID NO: 58 - DNA, 23 bp, Artificial Sequence
  Description of Artificial Sequence: Primer
accaaaagggcaatatggagcag

SEQ ID NO: 59 - skipped

SEQ ID NO: 60 - DNA, 25 bp, Artificial Sequence
  Description of Artificial Sequence: Primer
tcgttatgcctccattcggtccaac

SEQ ID NO: 61 - DNA, 26 bp, Artificial Sequence
  Description of Artificial Sequence: Primer
tgaagtccagcacattccagccactg

SEQ ID NO: 62 - DNA, 28 bp, Artificial Sequence
  Description of Artificial Sequence: Primer
agcgtcctggtggaataagaagccgtag

SEQ ID NO: 63 - DNA, 530 bp, Homo sapiens
  modified_base (154): a, t, c , g, unknown or other
aaaagtcttttgcggctgcagcgggcttgtaggtgtccggctttgctggcccagcaagcctgataagcatgaagctcttatctttggtggctgtggtcgggtgtttgctggtgcccccagcatgaagccaacaagagttctgaaaatatccggngcaaatgcatctgtccaccttatagaaacatcagagggcacatttacaaccagaatgtatcccagaaggactgcaactgcctgcacgtggtggagcccatgccagtgcctggccatgacgtggaggcctactgcctgctgtgcgagtgcaggtacgaggagcgcagcaccaccaccatcaaggtcatcattgtcatctacctgtccgtggtgggtgccctgttgctctacatggccttcctgatgctggtggaccctctgatccgaaagccggatgcatacactgagcaactgcacaatgaggaggagaatgaggagaagctggagtatttcttcctcattgtcttctcgattgaagccgccatgaagatcattgc

SEQ ID NO: 64 - protein, 47 aa, Homo sapiens
EKLEYFFLIVFSIEAAMKIIAYGFLFHQDAYLRSGWNVLDFTIVFLG

SEQ ID NO: 65 - protein, 133 aa, Homo sapiens
MEPSSPQDEGLRKKQPKKPVPEILPRPPRALFCLTLENPLRKACISIVEWKPFETIILLTIFANCVALAVYLPMPEDDNNSLNLGLEKLEYFFLIVFSIEAAMKIIAYGFLFHQDAYLRSGWNVLDFTIVFLG

SEQ ID NO: 66 - DNA, 284 bp, Homo sapiens
  modified_base (210): a, t, c, g, unknown or other
gaaaaaaaaaaaaaaaaaacactgtcatgccgttacgtagcgtatcgttgcagcgggcttgtaggtgtccggctttgctggcccagcaagcctgataagcatggagctcttatctttggtggctgtggtcgggtgtttgctggtgcccccagctgaagccaacaagagttctgaagatatccggtgcaaatgcatctgtccaccttatanaaacatcaagtgggcacaattacaaccagaatgtatcccagaaggactgcaactgcctgcacgtggtggagccc

SEQ ID NO: 67 - DNA, 252 bp, Homo sapiens
  modified_base (154): a, t, c, g, unknown or other
aaaagtcttttgcggctgcagcgggcttgtaggtgtccggctttgctggcccagcaagcctgataagcatgaagctcttatctttggtggctgtggtcgggtgtttgctggtgcccccagcatgaagccaacaagagttctgaaaatatccggngcaaatgcatctgtccaccttatagaaacatcagagggcacatttacaaccagaatgtatcccagaaggactgcaactgcctgcacgtggtggagccc

SEQ ID NO: 68 - DNA, 285 bp, Artificial Sequence
  Description of Artificial Sequence: Consensus sequence
  modified_base (211): a, t, c, g, unknown or other
gaaaaaaaaaaaaaaaaaacactgtcatgccgwwamgtmkyktrysgytgcagcgggcttgtaggtgtccggctttgctggcccagcaagcctgataagcatgragctcttatctttggtggctgtggtcgggtgtttgctggtgcccccagcatgaagccaacaagagttctgaaratatccggtgcaaatgcatctgtccaccttatanaaacatcaagtgggcacawttacaaccagaatgtatcccagaaggactgcaactgcctgcacgtggtggagccc

SEQ ID NO: 69 - protein, 32 aa, Homo sapiens
SEIDTFLASSGGLYCLGGGCGNVDPDESARIS

SEQ ID NO: 70 - DNA, 16 bp, Artificial Sequence
  Description of Artificial Sequence: Primer
ccggctgtgctccgag

SEQ ID NO: 71 - DNA, 20 bp, Artificial Sequence
  Description of Artificial Sequence: Primer
ttgctgagggaggtggaact

SEQ ID NO: 72 - DNA, 25 bp, Artificial Sequence
  Description of Artificial Sequence: Probe
tcagcccccagacccgaacactatt

SEQ ID NO: 73 - DNA, 20 bp, Artificial Sequence
  Description of Artificial Sequence: Primer
gcagacaatcagcggaacgt

SEQ ID NO: 74 - DNA, 23 bp, Artificial Sequence
  Description of Artificial Sequence: Primer
cgtcagcatcactgggatatgta

SEQ ID NO: 75 - DNA, 24 bp, Artificial Sequence
  Description of Artificial Sequence: Probe
agcccgggttttccttcgacagaa

SEQ ID NO: 76 - DNA, 20 bp, Artificial Sequence
  Description of Artificial Sequence: Primer
tgacgaaaatcggcaactga

SEQ ID NO: 77 - DNA, 20 bp, Artificial Sequence
  Description of Artificial Sequence: Primer
ggaaacccctcttcggagat

SEQ ID NO: 78 - DNA, 26 bp, Artificial Sequence
  Description of Artificial Sequence: Probe
cccagaggaggacaagagggacatcc

SEQ ID NO: 79 - DNA, 21 bp, Artificial Sequence
  Description of Artificial Sequence: Primer
ccaggcagaaacatcgactct

SEQ ID NO: 80 - DNA, 21 bp, Artificial Sequence
  Description of Artificial Sequence: Primer
cgagtcatcgtcctccaagaa

SEQ ID NO: 81 - DNA, 23 bp, Artificial Sequence
  Description of Artificial Sequence: Probe
cccgaggctaccatcatccccaa

SEQ ID NO: 82 - DNA, 18 bp, Artificial Sequence
  Description of Artificial Sequence: Primer
caccgtcccctgcatgtc

SEQ ID NO: 83 - DNA, 22 bp, Artificial Sequence
  Description of Artificial Sequence: Primer
tgtttcctccagcaggaaattc

SEQ ID NO: 84 - DNA, 28 bp, Artificial Sequence
  Description of Artificial Sequence: Probe
aaggccttcaattccacctgcaagtgat

SEQ ID NO: 85 - DNA, 23 bp, Artificial Sequence
  Description of Artificial Sequence: Primer
gacagaaggcaaggagtcctctt

SEQ ID NO: 86 - DNA, 19 bp, Artificial Sequence
  Description of Artificial Sequence: Primer
tcagtgggcatggcttcat

SEQ ID NO: 87 - DNA, 23 bp, Artificial Sequence
  Description of Artificial Sequence: Probe
tctgccagccaggaacgcagtct

SEQ ID NO: 88 - DNA, 21 bp, Artificial Sequence
  Description of Artificial Sequence: Primer
caaagggcaaaacaagcaaga

SEQ ID NO: 89 - DNA, 21 bp, Artificial Sequence
  Description of Artificial Sequence: Primer
tccctgcctgctcatctaggt

SEQ ID NO: 90 - DNA, 28 bp, Artificial Sequence
  Description of Artificial Sequence: Probe
aggatgaggaagtccctgatcggctttc

SEQ ID NO: 91 - DNA, 19 bp, Artificial Sequence
  Description of Artificial Sequence: Primer
aggtgtatcccaccgtgca

SEQ ID NO: 92 - DNA, 22 bp, Artificial Sequence
  Description of Artificial Sequence: Primer
gcagccacctctactagtgcct

SEQ ID NO: 93 - DNA, 22 bp, Artificial Sequence
  Description of Artificial Sequence: Probe
cctccaccggagacgctgaagg

SEQ ID NO: 94 - DNA, 19 bp, Artificial Sequence
  Description of Artificial Sequence: Primer
ccccagctttgcctttgag

SEQ ID NO: 95 - DNA, 20 bp, Artificial Sequence
  Description of Artificial Sequence: Primer
gcactatggcccctgaagag

SEQ ID NO: 96 - DNA, 27 bp, Artificial Sequence
  Description of Artificial Sequence: Probe
ttcttggacggtagccacagtgtgacc

SEQ ID NO: 97 - DNA, 18 bp, Artificial Sequence
  Description of Artificial Sequence: Primer
gggagacctgggcgaatg

SEQ ID NO: 98 - DNA, 25 bp, Artificial Sequence
  Description of Artificial Sequence: Primer
atctcacacaggaagttctctggat

SEQ ID NO: 99 - DNA, 24 bp, Artificial Sequence
  Description of Artificial Sequence: Probe
tcttccccttgtcctctacggccg

SEQ ID NO: 100 - DNA, 20 bp, Artificial Sequence
  Description of Artificial Sequence: Primer
tccaagggtctcccagacaa

SEQ ID NO: 101 - DNA, 20 bp, Artificial Sequence
  Description of Artificial Sequence: Primer
ttgggtttctgctccagctt

SEQ ID NO: 102 - DNA, 27 bp, Artificial Sequence
  Description of Artificial Sequence: Probe
cagaagaggagaagtcaacgatggcca

SEQ ID NO: 103 - DNA, 20 bp, Artificial Sequence
  Description of Artificial Sequence: consensus sequence
aactgtgagctggacaagaa

SEQ ID NO: 104 - DNA, 20 bp, Artificial Sequence
  Description of Artificial Sequence: Synthetic oligonucleotide
aactgtgagctggacaaaaa

SEQ ID NO: 105 - DNA, 26 bp, Artificial Sequence
  Description of Artificial Sequence: Primer
caggcgaagactggaatgctgtgatg

SEQ ID NO: 106 - DNA, 26 bp, Artificial Sequence
  Description of Artificial Sequence: Primer
atgctgtgatgtacgatggcatcatg

SEQ ID NO: 107 - DNA, 26 bp, Artificial Sequence
  Description of Artificial Sequence: Primer
atgctgtgatgtacgatggcatcatg

SEQ ID NO: 108 - DNA, 25 bp, Artificial Sequence
  Description of Artificial Sequence: Primer
tagatgaagaacagcatggctatga

SEQ ID NO: 109 - DNA, 20 bp, Artificial Sequence
  Description of Artificial Sequence: Primer
aactgtgagctggacaagaa

SEQ ID NO: 110 - DNA, 20 bp, Artificial Sequence
  Description of Artificial Sequence: Primer
aacaacaacttccagacctt

SEQ ID NO: 111 - DNA, 19 bp, Artificial Sequence
  Description of Artificial Sequence: Primer
aagatgttcaatgatgcca

SEQ ID NO: 112 - DNA, 19 bp, Artificial Sequence
  Description of Artificial Sequence: Primer
aagatgttcaatgatgcca

SEQ ID NO: 113 - DNA, 19 bp, Artificial Sequence
  Description of Artificial Sequence: Primer
aataggaacaataacttcc

SEQ ID NO: 114 - DNA, 21 bp, Artificial Sequence
  Description of Artificial Sequence: Primer
catcatgatggaaccgctgtt

SEQ ID NO: 115 - DNA, 22 bp, Artificial Sequence
  Description of Artificial Sequence: Primer
catcttcagggaatgggatgta

SEQ ID NO: 116 - DNA, 297 bp, Homo sapiens
gggtattttagtgacgcctggaacacgtttgactccctcatcgtaatcggcagcattatagacgtggccctcagcgaagcagaccactatttcactgatgcatggaacacttttgatgccttaattgttgttggtagcgtcgttgatattgctataactgaagtgaataactctgaagagagcaatagaatctccatcacctttttccgtcttttccgagtgatgcgattggtgaagcttctcagcaggggggaaggcatccggacattgctgtggacttttattaagtcctttcag

SEQ ID NO: 117 - protein, 99 aa, Homo sapiens
GYFSDAWNTFDSLIVIGSIIDVALSEADHYFTDAWNTFDALIVVGSVVDIAITEVNNSEESNRISITFFRLFRVMRLVKLLSRGEGIRTLLWTFIKSFQ

SEQ ID NO: 118 - DNA, 254 bp, Homo sapiens
gaagcttttatactgcaacaaacaatacttgtgctttccctccatctagcactgtgcctccaggagagagggtttttttctggtcgcccctgcagacatgaccctgcttgtccctggccaatcccagggccagactggattgttggccctctgactcaccctagaaggcctggggctggagaggcctggaactcctcggagagttgagtgctccaaacagaatcaaactccaatgaataacaagtaattttgga

SEQ ID NO: 119 - DNA, 400 bp, Homo sapiens
gaaaaaacaatacttgtgctttccctccatctagcactgtgcctccaggagagagggtttttttttctggtcgcccctgcagacatgaccctgcttgtccctggccaatcccagggccagactggattgttggccctctgactcaccctagaaggcctggggctggagaggcctggaactcctcggagaagccaaagatttgcagaatcaaggatggatggagtatcaaaataaggaacggaaaaaactgaagatatactaaggattaaggcccaggttcatctagtgtccccaggtgccaggcattgtgctgtgactgtgatgtgaaaaaagaggcaggacaactgagttgagtgctccaaacagaatcaaactccaatgaataacaagtaattttgga

SEQ ID NO: 120 - DNA, 302 bp, Homo sapiens
gccctctagatgcatgctcgagcggccgccagtgtgatggatatctgcagaattcgcccttggacactgacatggactgaaggagtagaaatctagcactgtgcctccaggagagagggttttttttctggtcgcccctgcagacatgaccctgcttgtccctggccaatcccagggccagactggattgttggccctctgactcaccctagaaggcctggggctggagaggcctggaactcctcggagagttgagtgctccaaacagaatcaaactccaatgaataacaagtaattttgga

SEQ ID NO: 121 - DNA, 465 bp, Artificial Sequence
  Description of Artificial Sequence: Consensus sequence
gccctctagatgcatgctcgagcggccgccagtgtgatggatatctgmagmwttyrymctkgaacaaacacatacttgtgctttccctccatctagcactgtgcctccaggagagagggtttttttttctggtcgcccctgcagacatgaccctgcttgtccctggccaatcccagggccagactggattgttggccctctgactcaccctagaaggcctggggctggagaggcctggaactcctcggagagttgagtgctccaaacagaatcaaactccaatgaataacaagtaattttggacggaaaaaactgaagatatactaaggattaaggcccaggttcatctagtgtccccaggtgccaggcattgtgctgtgactgtgatgtgaaaaaagaggcaggacaactgagttgagtgctccaaacagaatcaaactccaatgaataacaagtaattttgga

SEQ ID NO: 122 - DNA, 6221 bp, Homo sapiens
  modified_base (6030): a, t, c , g, unknown or other
  modified_base (6042): a, t, c , g, unknown or other
  modified_base (6067): a, t, c , g, unknown or other
  modified_base (6073): a, t, c , g, unknown or other
  modified_base (6084): a, t, c , g, unknown or other
  modified_base (6096): a, t, c , g, unknown or other
  modified_base (6131): a, t, c , g, unknown or other
  modified_base (6133): a, t, c , g, unknown or other
  modified_base (6135): a, t, c , g, unknown or other
  modified_base (6141): a, t, c , g, unknown or other
  modified_base (6146): a, t, c , g, unknown or other
  modified_base (6166): a, t, c , g, unknown or other
gaagcttttatactgcaacaaacaatacttgtgctttccctccatctagcactgtgcctccaggagagagggtttttttctggtcgcccctgcagacatgaccctgcttgtccctggccaatcccagggccagactggattgttggccctctgactcaccctagaaggcctggggctggagaggcctggaactcctcggagagttgagtgctccaaacagaatcaaactccaatgaataacaagtaattttggaatgttttacatcatgatggaaccgctgtttaaatgcaggaaaacaagctccaggctcccactgattctacatgatgaggcaaactatgcaagaggcaccagacttcctctttctggtgaaggaccaacttctcagccgaatagctccaagcaaactgtcctgtcttggcaagctgcaatcgatgctgctagacaggccaaggctgcccaaactatgagcacctctgcacccccacctgtaggatctctctcccaaagaaaacgtcagcaatacgccaagagcaaaaaacagggtaactcgtccaacagccgacctgcccgcgcccttttctgtttatcactcaataaccccatccgaagagcctgcattagtatagtggaatggaaaccatttgacatatttatattattggctatttttgccaattgtgtggccttagctatttacatcccattccctgaagatgattctaattcaacaaatcataacttggaaaaagtagaatatgccttcctgattatttttacagtcgagacatttttgaagattatagcgtatggattattgctacatcctaatgcttatgttaggaatggatggaatttactggattttgttatagtaatagtaggattgtttagtgtaattttggaacaattaaccaaagaaacagaaggcgggaaccactcaagcggcaaatctggaggctttgatgtcaaagccctccgtgcctttcgagtgttgcgaccacttcgactagtgtcaggggtgcccagtttacaagttgtcctgaactccattataaaagccatggttcccctccttcacatagcccttttggtattatttgtaatcataatctatgctattataggattggaactttttattggaaaaatgcacaaaacatgtttttttgctgactcagatatcgtagctgaagaggacccagctccatgtgcgttctcagggaatggacgccagtgtactgccaatggcacggaatgtaggagtggctgggttggcccgaacggaggcatcaccaactttgataactttgcctttgccatgcttactgtgtttcagtgcatcaccatggagggctggacagacgtgctctactgggtaaatgatgcgataggatgggaatggccatgggtgtattttgttagtctgatcatccttggctcatttttcgtccttaacctggttcttggtgtccttagtggagaattctcaaaggaaagagagaaggcaaaagcacggggagatttccagaagctccgggagaagcagcagctggaggaggatctaaagggctacttggattggatcacccaagctgaggacatcgatccggagaatgaggaagaaggaggagaggaaggcaaacgaaatactagcatgcccaccagcgagactgagtctgtgaacacagagaacgtcagcggtgaaggcgagaaccgaggctgctgtggaagtctctgtcaagccatctcaaaatccaaactcagccgacgctggcgtcgctggaaccgattcaatcgcagaagatgtagggccgccgtgaagtctgtcacgttttactggctggttatcgtcctggtgtttctgaacaccttaaccatttcctctgagcactacaatcagccagattggttgacacagattcaagatattgccaacaaagtcctcttggctctgttcacctgcgagatgctggtaaaaatgtacagcttgggcctccaagcatatttcgtctctcttttcaaccggtttgattgcttcgtggtgtgtggtggaatcactgagacgatcttggtggaactggaaatcatgtctcccctggggatctctgtgtttcggtgtgtgcgcctcttaagaatcttcaaagtgaccaggcactggacttccctgagcaacttagtggcatccttattaaactccatgaagtccatcgcctcgctgttgcttctgctttttctcttcattatcatcttttccttgcttgggatgcagctgtttggcggcaagtttaattttgatgaaacgcaaaccaagcggagcacctttgacaatttccctcaagcacttctcacagtgttccagatcctgacaggcgaagactggaatgctgtgatgtacgatggcatcatggcttacgggggcccatcctcttcaggaatgatcgtctgcatctacttcatcatcctcttcatttgtggtaactatattctactgaatgtcttcttggccatcgctgtagacaatttggctgatgctgaaagtctgaacactgctcagaaagaagaagcggaagaaaaggagaggaaaaagattgccagaaaagagagcctagaaaataaaaagaacaacaaaccagaagtcaaccagatagccaacagtgacaacaaggttacaattgatgactatagagaagaggatgaagacaaggacccctatccgccttgcgatgtgccagtaggggaagaggaagaggaagaggaggaggatgaacctgaggttcctgccggaccccgtcctcgaaggatctcggagttgaacatgaaggaaaaaattgcccccatccctgaagggagcgctttcttcattcttagcaagaccaacccgatccgcgtaggctgccacaagctcatcaaccaccacatcttcaccaacctcatccttgtcttcatcatgctgagcagcgctgccctggccgcagaggaccccatccgcagccactccttccggaacacgatactgggttactttgactatgccttcacagccatctttactgttgagatcctgttgaagatgacaacttttggagctttcctccacaaaggggccttctgcaggaactacttcaatttgctggatatgctggtggttggggtgtctctggtgtcatttgggattcaatccagtgccatctccgttgtgaagattctgagggtcttaagggtcctgcgtcccctcagggccatcaacagagcaaaaggacttaagcacgtggtccagtgcgtcttcgtggccatccggaccatcggcaacatcatgatcgtcaccaccctcctgcagttcatgtttgcctgtatcggggtccagttgttcaaggggaagttctatcgctgtacggatgaagccaaaagtaaccctgaagaatgcaggggacttttcatcctctacaaggatggggatgttgacagtcctgtggtccgtgaacggatctggcaaaacagtgatttcaacttcgacaacgtcctctctgctatgatggcgctcttcacagtctccacgtttgagggctggcctgcgttgctgtataaagccatcgactcgaatggagagaacatcggcccaatctacaaccaccgcgtggagatctccatcttcttcatcatctacatcatcattgtagctttcttcatgatgaacatctttgtgggctttgtcatcgttacatttcaggaacaaggagaaaaagagtataagaactgtgagctggacaaaaatcagcgtcagtgtgttgaatacgccttgaaagcacgtcccttgcggagatacatccccaaaaacccctaccagtacaagttctggtacgtggtgaactcttcgcctttcgaatacatgatgtttgtcctcatcatgctcaacacactctgcttggccatgcagcactacgagcagtccaagatgttcaatgatgccatggacattctgaacatggtcttcaccggggtgttcaccgtcgagatggttttgaaagtcatcgcatttaagcctaaggggtattttagtgacgcctggaacacgtttgactccctcatcgtaatcggcagcattatagacgtggccctcagcgaagcagaccactatttcactgatgcatggaacacttttgatgccttaattgttgttggtagcgtcgttgatattgctataactgaagtgaataactctgaagagagcaatagaatctccatcacctttttccgtcttttccgagtgatgcgattggtgaagcttctcagcaggggggaaggcatccggacattgctgtggacttttattaagtcctttcaggcgctcccgtatgtggccctcctcatagccatgctgttcttcatctatgcggtcattggcatgcagatgtttgggaaagttgccatgagagataacaaccagatcaataggaacaataacttccagacgtttccccaggcggtgctgctgctcttcaggtgtgcaacaggtgaggcctggcaggagatcatgctggcctgtctcccagggaagctctgtgaccctgagtcagattacaaccccggggaggagtatacatgtgggagcaactttgccattgtctatttcatcagtttttacatgctctgtgcatttctgatcatcaatctgtttgtggctgtcatcatggataatttcgactatctgacccgggactggtctattttggggcctcaccatttagatgaattcaaaagaatatggtcagaatatgaccctgaggcaaagggaaggataaaacaccttgatgtggtcactctgcttcgacgcatccagcctcccctggggtttgggaagttatgtccacacagggtagcgtgcaagagattagttgccatgaacatgcctctcaacagtgacgggacagtcatgtttaatgcaaccctgtttgctttggttcgaacggctcttaagatcaagaccgaagggaacctggagcaagctaatgaagaacttcgggctgtgataaagaaaatttggaagaaaaccagcatgaaattacttgaccaagttgtccctccagctggtgatgacgaggtaaccgtggggaagttctatgccactttcctgatacaggactactttaggaaattcaagaaacggaaagaacaaggactggtgggaaagtaccctgcgaagaacaccacaattgccctacaggcgggattaaggacactgcatgacattgggccagaaatccggcgtgctatatcgtgtgatttgcaagatgacgagcctgaggaaacaaaacgagaagaagaagatgatgtgttcaaaagaaatggtgccctgcttggaaaccatgtcaatcatgttaatagtgataggagagattcccttcagcagaccaataccacccaccgtcccctgcatgtccaaaggccttcaattccacctgcaagtgatactgagaaaccgctgtttcctccagcaggaaattcggtgtgtcataaccatcataaccataattccataggaaagcaagttcccacctcaacaaatgccaatctcaataatgccaatatgtccaaagctgcccatggaaagcggcccagcattgggaaccttgagcatgtgtctgaaaatgggcatcattcttcccacaagcatgaccgggagcctcagagaaggtccagtgtgaaaaggtaaccttgacaatgtgtttggacttgctcatgtggtgtctgcccgtgttgcgtctagtcccaaggagcaaggcgggtctgtgctctgtgctctggggcatggaggaggttccccgtaggcatttgtgtcaggatctgtgagtgccccccaggtcactgctgcctctgacactctccacgtgatggacctaaaggataagggccacgtgccatcctacatcctgtgtggggtgccgagggaccttwaaacagcagcagccacaaaatccacctctcaattgtgctcacatactgtgcgacaggggttggtgaattgtttntttaaagggtcnctgagtcaatattttaggctttganaaccanacagttttgcnccttttcagctntgccgttgcagtgtgaaatatgtaagtgaatgggngngnggctgngcccnttaaaatttccctatggtcnctgaaatttgaacttcaaaaaaaaaaaaaaaaaaaaaaaaaaaaaaaaaaaaaaa

SEQ ID NO: 123 - protein, 25 aa, Homo sapiens
MFYIMMEPLFRCRKTSSRLPLILHD

SEQ ID NO: 124 - protein, 21 aa, Homo sapiens
MMMMMMMKKMQHQRQQQADHA

SEQ ID NO: 125 - protein, 25 aa, Artificial Sequence
  Description of Artificial Sequence: Consensus sequence
  MOD_RES (7): Met or Glu
  MOD_RES (8): Met or Pro
  MOD_RES (9): Met or Leu
  MOD_RES (10): Met or Phe
  MOD_RES (11): Met or Arg
  MOD_RES (12): Cys or Lys
  MOD_RES (13): Arg or Lys
  MOD_RES (14): Met or Lys
  MOD_RES (15): Thr or Gln
  MOD_RES (16): Ser or His
  MOD_RES (17): Ser or Gln
  MOD_RES (19): Leu or Gln
  MOD_RES (20): Pro or Gln
  MOD_RES (21): Leu or Gln
  MOD_RES (22): Ile or Ala
  MOD_RES (23): Leu or Asp
  MOD_RES (25): Asp or Ala
MFYIMMXXXXXXXXXXXRXXXXXHX

SEQ ID NO: 126 - protein, 97 aa, Homo sapiens
VLISEGLGRYARDPKFVSATKHEIADACDLTIDEMESAASTLLNGNVRPRANGDVGPLSHRQDYELQDFGPGYSDEEPDPGRDEEDLADEMICITTL